(19) United States
(12) Patent Application Publication
(10) Pub. No.: US 2018/0333382 A1
(43) Pub. Date: Nov. 22, 2018

(54) PHARMACEUTICAL COMPOSITIONS COMPRISING DGLA AND USE OF SAME

(71) Applicant: DS Biopharma Limited, Dublin (IE)

(72) Inventors: John Climax, Dublin (IE); Mehar Manku, Birmingham (GB); David Coughlan, Dublin (IE)

(21) Appl. No.: 15/982,599

(22) Filed: May 17, 2018

Related U.S. Application Data
(60) Provisional application No. 62/508,818, filed on May 19, 2017.

(51) Int. Cl. A61K 31/202; A61K 9/00; A61K 9/48; A61P 11/02; A61P 11/06; A61P 11/08; A61P 17/04

(52) U.S. Cl. CPC A61K 31/202; A61K 9/0053; A61K 9/48; A61P 17/04; A61P 11/06; A61P 11/08; A61P 11/02

(57) ABSTRACT

The present disclosure provides orally deliverable pharmaceutical compositions comprising DGLA and methods of using same to treat a variety of conditions and disorders.

### PRIORITY CLAIM

This application claims priority to U.S. Provisional Patent Application Ser. No. 62/508,818, filed on May 19, 2017, the entire contents of which is incorporated herein by reference.

### TECHNICAL FIELD

The present application relates generally to pharmaceutical compositions comprising DGLA and methods of using same.

### BACKGROUND

Dihomo gamma linolenic acid (DGLA) is an essential fatty acid found naturally in the body as the elongation product of gamma linolenic acid (GLA). GLA is, in turn, a desaturation product of linoleic acid. Soft gelatin encapsulation of DGLA is challenging as it is prone to oxidation to aldehydes, which can interact with amino groups in the gelatin polymer in the capsule shell. This can cause slowdown in drug release due to crosslinking of the gelatin polymers.

### SUMMARY

The present disclosure provides orally deliverable pharmaceutical compositions comprising DGLA and methods of using same to treat a variety of conditions and disorders.

In one aspect, the present disclosure provides methods of treating atopic dermatitis in a subject having moderate to severe atopic dermatitis and a normal eosinophil count, the method comprising orally administering to the subject a pharmaceutical composition comprising DGLA or a derivative thereof.

In another aspect, the present disclosure provides methods of reducing at least one of an investigator global assessment (IGA) level, eczema area and severity index (EASI), a percentage of area of an anatomical site affected by atopic dermatitis, a scoring atopic dermatitis (SCORAD), a body surface area affected by atopic dermatitis, a dermatology life quality Index (DLQI), a patient-oriented eczema measure (POEM), or visual analog scale (VAS) pruritus score in a subject in need thereof and having an eosinophil count of less than 0.3×10⁹/L, the method comprising orally administering to the subject a pharmaceutical composition comprising DGLA or derivative thereof.

In yet another aspect, the present disclosure provides pharmaceutical compositions comprising DGLA or a derivative thereof for the treatment of a condition wherein a subset of patients having the condition have a normal eosinophil count as defined by a laboratory reference range.

In still another aspect, the present disclosure provides for the use of a pharmaceutical composition comprising DGLA or a derivative thereof for treatment of one or more conditions in a subject having an eosinophil count of less than about 0.3×10⁹/L.

In one embodiment, the pharmaceutical composition is administered in an amount sufficient to provide about 2 g of DGLA, or the derivative thereof, per day for a period of at least about 8 weeks. In another embodiment, the pharmaceutical composition is administered in an amount sufficient to provide up to 4 g of DGLA or a derivative thereof for a period of at least about 8 weeks. In yet another embodiment, the pharmaceutical composition is administered in an amount sufficient to provide about 0.2 to about 3 g of DGLA or derivative thereof to the subject per day. In still yet another embodiment, the pharmaceutical composition is administered in an amount sufficient to provide about 0.5 g, about 1 g or about 2 g of DGLA or derivative thereof to the subject per day. In a further embodiment, the pharmaceutical composition is administered in an amount sufficient to provide about 2 g of DGLA or derivative thereof to the subject per day.

In one embodiment, the DGLA or a derivative thereof is administered to the subject for a period of at least about 2 weeks, at least about 4 weeks, or at least about 8 weeks.

In one embodiment, the subject's EASI score decreases by about 30%, about 40%, about 50%, about 60%, about 70% or more than about 70% compared to baseline.

In one embodiment, the subject's VAS score decreases by about 30%, about 40%, about 50%, about 60% or more than about 60% compared to baseline.

In one embodiment, the subject's SCORAD score decreases by about 30%, about 40%, about 50%, about 60%, about 70% or more than about 70% compared to baseline.

In one embodiment, the subject's IGA score decreases by about 30%, about 40%, about 50%, about 60% or more than about 60% compared to baseline.

In one embodiment, in a sample of patients, the proportion of IGA responders is between 20% to 70%. A patient is deemed as an IGA responder if his/her IGA score decreases by 2 or more points from baseline and he/she has a score of 0 or 1 at the end of a period of treatment.

In one embodiment, the subject's body surface area affected by atopic dermatitis is assigned a body surface area (BSA) score. In another embodiment, the subject's BSA score decreases by about 30%, about 40%, about 50%, about 60% or more than about 60% compared to baseline.

In one embodiment, the subject's DLQI score decreases by about 40%, about 50%, about 60%, about 70%, or more than about 70% compared to baseline.

In one embodiment, the subject's POEM score decreases by about 40%, about 50%, about 60%, about 70%, or more than about 70% compared to baseline.

In one embodiment, the pruritus level and/or a pruritus delta are calculated from the VAS. In another embodiment, the subject's pruritus level is reduced by about 40%, about 50%, about 60%, about 70%, about 80%, about 90% or more than about 90% compared to baseline. In yet another embodiment, the subject's pruritus delta is about 30%, about 40%, about 50%, about 60%, about 70%, or more than about 70%.

In one embodiment, the subject is a pediatric subject.

In one embodiment, the subject is an adult subject.

In one embodiment, the subject has an eosinophil count that is determined based on a comparison to a laboratory reference level.

In one embodiment, the normal eosinophil count is less than about 0.2×10⁹/L, less than about 0.25×10⁹/L, less than about 0.3×10⁹/L, less than about 0.4×10⁹/L, less than about 0.5×10⁹/L, less than 0.6×10⁹/L, less than 0.7×10⁹/L or less than about 0.8×10⁹/L.

In one embodiment, the condition is asthma, atopic dermatitis, chronic rhinosinusitis, or a combination thereof.

In one embodiment, the condition is chronic obstructive pulmonary disease (COPD), asthma, atopic dermatitis, chronic rhinosinusitis, or a combination thereof.

In one embodiment, chronic rhinosinusitis includes chronic sinusitis with nasal polyps (CRSwNP).

In one embodiment, chronic sinusitis with nasal polyps (CRSwNP) include a non-eosinophilic subtype.

In one embodiment, atopic dermatitis includes atopic dermatitis having any EASI, VAS, SCORAD, IGA, BSA, DLQI, and/or POEM score.

In one embodiment, the one or more conditions are atopic conditions.

In one embodiment, the atopic conditions include atopic dermatitis.

In one embodiment, the one or more conditions include chronic obstructive pulmonary disease (COPD).

In one embodiment, the one or more conditions include vasculitides.

In one embodiment, vasculitides includes large vessel vasculitis (LVV), medium vessel vasculitis (MW), small vessel vasculitis (SW), variable vessel vasculitis (VVV), vingle-organ vasculitis (SOV) and vasculitis associated with systemic disease.

In one embodiment, the subject has eosinophil levels that are greater than normal eosinophil levels, less than normal eosinophil levels, or a combination thereof.

In one embodiment, the pharmaceutical composition comprises DGLA or a derivative thereof in a liquid or semi-liquid format.

In one embodiment, the present disclosure provides a pharmaceutical composition comprising DGLA. In one embodiment, the composition is encapsulated in a capsule shell. In one embodiment, the present disclosure provides a pharmaceutical composition comprising DGLA encapsulated in a capsule shell comprising gelatin, d-sorbitol and 1,4-sorbitan sugar alcohols. In one embodiment, about 500 mg to about 1 g of DGLA or a derivative thereof is encapsulated in the capsule shell. In another embodiment, about 200 mg to about 1 g of DGLA or a derivative thereof is encapsulated in the capsule shell.

These and other embodiments of the invention are described in further detail below.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1 shows changes in eczema area and severity index from baseline for DGLA and placebo.

FIG. 2 shows changes in visual analog scale of pruritus from baseline for DGLA and placebo.

FIG. 3 shows changes in Scoring atopic dermatitis (SCORAD) from baseline for DGLA and placebo.

FIG. 4 shows changes in body surface area affected by atopic dermatitis for DGLA and placebo.

FIG. 5 shows total population changes in Investigator's Global Assessment (IGA) score at week 8.

FIG. 6 shows moderate population changes in IGA score at week 8.

FIG. 7 shows change in mean arterial pressure (mmHg) with intravenous doses of phenylephrine following seven consecutive days of gavage with aspirin at 10 mg/kg/day.

FIG. 8 shows change in mean arterial pressure (mmHg) with intravenous doses of phenylephrine following seven consecutive days of gavage with DGLA at 50 mg/kg+aspirin at 10 mg/kg.

FIG. 9 shows change in mean arterial pressure (mmHg) with intravenous doses of phenylephrine following seven consecutive days of gavage with DGLA at 500 mg/kg co-administered with aspirin at 10 mg/kg.

FIG. 10 shows mean arterial pressure at baseline following seven consecutive days with six different gavage groups.

FIG. 11 shows mean arterial pressure with an intravenous dose of phenylephrine at 20 μk/kg following seven consecutive days with six different gavage groups.

FIG. 12 shows mean plasma free DGLA concentration (ng/mL, linear plot), by dose cohort (Single Dose, PK Population).

FIG. 13 shows mean plasma free DGLA concentration (ng/mL, log-linear plot), by dose cohort (Single Dose, PK Population).

FIG. 14 shows mean plasma total DGLA concentration (ng/mL, linear plot), by dose cohort (Single Dose, PK Population).

FIG. 15 shows mean plasma total DGLA concentration (ng/mL, log-linear plot), by dose cohort (Single Dose, PK Population).

FIG. 16 shows mean plasma free DGLA concentration (ng/mL, linear plot), by dose cohort (Multiple-dose, PK Population).

FIG. 17 shows mean plasma free DGLA Concentration (ng/mL, log-linear plot), by dose cohort (Multiple-dose, PK Population).

FIG. 18 shows mean plasma total DGLA concentration (ng/mL, linear plot), by dose cohort (Multiple-dose, PK Population).

FIG. 19 shows mean plasma total DGLA concentration (ng/mL, log-linear plot), by dose cohort (Multiple-dose, PK Population).

FIG. 20 shows mean skin blister fluid concentration of free DGLA (ng/mL, linear plot), by dose cohort (Multiple-dose, PK Population).

FIG. 21 shows mean skin blister fluid concentration of free DGLA (ng/mL, log-linear plot), by dose cohort (Multiple-dose, PK Population).

FIG. 22 shows mean skin blister fluid concentration of total DGLA (ng/mL, linear plot), by dose cohort (Multiple-dose, PK Population).

FIG. 23 shows mean skin blister fluid concentration of total DGLA (ng/mL, log-linear plot), by dose cohort (Multiple-dose, PK Population)

FIG. 24 shows mean free DGLA concentration (ng/mL, linear plot) in plasma and skin blister fluid, by dose cohort (Multiple-dose, PK Population).

FIG. 25 shows mean free DGLA concentration (ng/mL, log-linear plot) in plasma and skin blister fluid, by dose cohort (Multiple-dose, PK Population).

FIG. 26 shows mean total DGLA concentration (ng/mL, linear plot) in plasma and skin blister fluid, by dose cohort (Multiple-dose, PK Population).

FIG. 27 shows mean total DGLA concentration (ng/mL, log-linear plot) in plasma and skin blister fluid, by dose cohort (Multiple-dose, PK Population).

FIG. 28 shows mean plasma dihydrotestosterone concentration (ng/mL, linear plot), by dose cohort (Multiple-dose, PK Population).

FIG. 29 shows mean plasma dihydrotestosterone concentration (ng/mL, log-linear plot), by dose cohort (Multiple-dose, PK Population).

FIGS. 30A-30C summarize eosinophil concentrations by ethnicity. FIG. 30A shows changes in eosinophil concentration (×10³ μg/ml) over an 8 week course of oral DGLA treatment. FIGS. 30B and 30C depict statistical results, at specific time points during DGLA treatment, for African American and Caucasian patients, respectively.

FIGS. 31A-31B show responders versus non-responders by ethnicity for oral DGLA treated patients (FIG. 31A) and placebo treated patients (FIG. 31B).

FIGS. 32A-32C summarize all responder data for African Americans and Caucasian responders (FIG. 32A), separated based on treatment (oral DGLA treated patients (FIG. 32B) and placebo treated patients (FIG. 32C).

FIGS. 33A-33C summarize eosinophil concentrations in Responders and Non-responders (total population). FIGS. 33B and 33C depict statistical results, at specific time points during DGLA treatment, for Responders and Non-responders, respectively.

FIGS. 34A-34C summarize eosinophil concentrations in oral DGLA treated Responders and Non-responders.

FIGS. 35A-35C summarize eosinophil concentrations in placebo treated Responders and Non-responders.

FIG. 36 shows percentages of IGA responders stratified by the primary study endpoint, moderate population, severe population and normal eosinophil population.

FIGS. 37A-37B show percentage change in IGA from baseline stratified by total study population (ITT, FIG. 37A) and participants having normal eosinophil levels (Normal Eosinophil, FIG. 37B) and separated based on treatment type (oral DGLA treated patients and placebo treated patients).

FIGS. 38A-38B show percentage change in EASI from baseline stratified by total study population (ITT, FIG. 38A) and participants having normal eosinophil levels (Normal Eosinophil, FIG. 38B) and separated based on treatment type (oral DGLA treated patients and placebo treated patients).

FIGS. 39A-39B show percentage change in pruritus VAS from baseline stratified by total study population (ITT, FIG. 39A) and participants having normal eosinophil levels (Normal Eosinophil, FIG. 39B) and separated based on treatment type (oral DGLA treated patients and placebo treated patients).

FIGS. 40A-40B show percentage change in SCORAD from baseline stratified by total study population (ITT, FIG. 40A) and participants having normal eosinophil levels (Normal Eosinophil, FIG. 40B) and separated based on treatment type (oral DGLA treated patients and placebo treated patients).

FIGS. 41A-41B show percentage change in BSA from baseline stratified by total study population (ITT, FIG. 41A) and participants having normal eosinophil levels (Normal Eosinophil, FIG. 41B) and separated based on treatment type (oral DGLA treated patients and placebo treated patients).

FIGS. 42A-42B show percentage change in DLQI from baseline stratified by total study population (ITT, FIG. 42A) and participants having normal eosinophil levels (Normal Eosinophil, FIG. 42B) and separated based on treatment type (oral DGLA treated patients and placebo treated patients).

FIGS. 43A-43B show percentage change in POEM from baseline stratified by total study population (ITT, FIG. 43A) and participants having normal eosinophil levels (Normal Eosinophil, FIG. 43B) and separated based on treatment type (oral DGLA treated patients and placebo treated patients).

### DETAILED DESCRIPTION

While the present invention is capable of being embodied in various forms, the description below of several embodiments is made with the understanding that the present disclosure is to be considered as an exemplification of the invention, and is not intended to limit the invention to the specific embodiments illustrated. Headings are provided for convenience only and are not to be construed to limit the invention in any manner. Embodiments illustrated under any heading may be combined with embodiments illustrated under any other heading.

The use of numerical values in the various quantitative values specified in this application, unless expressly indicated otherwise, are stated as approximations as though the minimum and maximum values within the stated ranges were both preceded by the word “about.” In this manner, slight variations from a stated value can be used to achieve substantially the same results as the stated value. Also, the disclosure of ranges is intended as a continuous range including every value between the minimum and maximum values recited, as well as any ranges that can be formed by such values. Also disclosed herein are any and all ratios (and ranges of any such ratios) that can be formed by dividing a recited numeric value into any other recited numeric value. Accordingly, the skilled person will appreciate that many such ratios, ranges, and ranges of ratios can be unambiguously derived from the numerical values presented herein; and, in all instances, such ratios, ranges, and ranges of ratios represent various embodiments of the present invention.

### Compositions

In various embodiments, the present disclosure provides orally deliverable pharmaceutical compositions comprising DGLA or a derivative thereof. The term DGLA herein refers to DGLA in free acid form. Compositions of the invention may also comprise a DGLA derivative in addition to or instead of DGLA. Such derivatives include alkyl esters; lower alky esters, such as DGLA methyl or ethyl ester; or DGLA in triglyceride form. In one embodiment, the present disclosure provides a pharmaceutical composition comprising DGLA or derivative thereof encapsulated in a capsule shell. In one embodiment, about 500 mg to about 1 g of DGLA or derivative thereof is encapsulated in the capsule shell.

In one embodiment, the capsule shell comprises gelatin (for example, Gelatin RXL or lime bone gelatin with a lower molecular weight). In another embodiment, the capsule shell comprises Gelatin RXL that has been treated by proteolytic enzyme to cut the gelatin pattern and effectively decrease its molecular weight. In another embodiment, the pharmaceutical composition comprises DGLA esters of D-Sorbitol and 1,4-sorbitan. In one embodiment, the capsule shell comprises (a) gelatin and (b) plasticizers selected from one or more of d-sorbitol and 1,4-sorbitans. In one embodiment, the gelatin is as described in U.S. Pat. No. 7,485,323, and is hereby incorporated by reference herein in its entirety.

In one embodiment, the plasticizer comprises 1,4-sorbitans in an amount from 20%-30%, for example, about 24% and 28% (on a dry basis), and a D-sorbitol content of about 30%-50%, for example, about 35% to 45% (on a dry basis).

In some embodiments, the capsule shell further comprises glycerol, purified water, titanium dioxide, medium chain triglycerides and lecithin.

In various embodiments, DGLA or a derivative is present in a composition of the invention in an amount of about 50 mg to about 5000 mg, about 75 mg to about 2500 mg, or about 100 mg to about 1000 mg, for example, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1025 mg, about 1050 mg, about 1075 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, about 1875 mg, about 1900 mg, about 1925 mg, about 1950 mg, about 1975 mg, about 2000 mg, about 2025 mg, about 2050 mg, about 2075 mg, about 2100 mg, about 2125 mg, about 2150 mg, about 2175 mg, about 2200 mg, about 2225 mg, about 2250 mg, about 2275 mg, about 2300 mg, about 2325 mg, about 2350 mg, about 2375 mg, about 2400 mg, about 2425 mg, about 2450 mg, about 2475 mg, or about 2500 mg. In any such embodiment, the composition can further comprise DGLA esters of D-Sorbitol and 1,4-sorbitan.

In one embodiment, a composition of the invention contains not more than about 10%, not more than about 9%, not more than about 8%, not more than about 7%, not more than about 6%, not more than about 5%, not more than about 4%, not more than about 3%, not more than about 2%, not more than about 1%, or not more than about 0.5%, by weight of total fatty acids, of fatty acids other than DGLA.

In one embodiment, a composition of the invention independently comprises not more than 15%, not more than 10%, not more than 5%, not more than 4%, not more than 3%, not more than 2% or not more than 1% of an one or more of the following fatty acids: C16:0, 16:3n-3, C18:0, C18:1n-7, C18:1n-9, C18:2n-6, C18:3n-6, 19:3, 20:1n-9, 20:2n-6, 20:2n-3+DGLA isomer, C20:3n-3, 20:4n-3, methyl 7,11,14-eicosatrienoate, C20:4n-6, C20:5n-3, C22:0, 22:5n-3 and/or C24:0.

In another embodiment, DGLA or a derivative thereof represents at least about 30%, about 40%, about 50%, at least about 60%, at least about 70%, at least about 80%, at least about 90%, at least about 95%, at least about 97%, at least about 98%, at least about 99%, or 100%, by weight, of all fatty acids present in a composition of the invention.

In one embodiment, a composition of the invention when placed in a standard disintegration test, for example, as set forth in USP 2040 (Disintegration and Dissolution of Dietary Supplements) with water as the Medium has a DGLA release rate less than about 60 minutes, less than about 50 minutes, less than about 40 minutes, less than about 30 minutes, or less than 20 minutes after storage for about 1 month, about 2 months, or about 3 months at 40° C./75% RH.

In one embodiment, after storage for about 1 month, about 2 months, about 3 months, or about 6 months at 40° C./75% RH, a composition of the invention comprises less than about 5% DGLA esters by weight of all fatty acids, less than about 4% DGLA esters by weight of all fatty acids, less than about 3% DGLA esters by weight of all fatty acids, less than about 2% DGLA esters by weight of all fatty acids, or less than about 1% DGLA esters by weight of all fatty acids.

### Methods

Any composition of the invention, including compositions described herein above or compositions that can for formulated from combining various embodiments of the present disclosure, can be used in treatment or prevention of: skin disorders and diseases, including acne vulgaris, acne rosacea, atopic dermatitis (e.g., atopic dermatitis having any EASI, VAS, SCORAD, IGA, BSA, DLQI, and/or POEM score), psoriasis, pruritus/itch, radiation protection, dry skin, smooth skin, healthy skin, anti-aging, photoprotection, and atopic conditions; urinary disorders and diseases including bladder cancer, cystocele, hematuria, interstitial cystitis, neurogenic bladder, Peyronie's disease, prostate disease, incontinence, urinary tract infection, and vasicoureteral reflux; renal disease and disorders including kidney failure, acute kidney injury, chronic kidney disease, and polycystic kidney disease; rheumatic disease including ankylosing spondylitis, fibromyalgia, gout, infectious arthritis, lupus, osteoarthritis, polymyalgia rheumatica, psoriatic arthritis, reactive arthritis, rheumatoid arthritis, sclerodoma; respiratory disorders including inflammatory lung disease, respiratory tract infections, pleural cavity disease, pulmonary vascular disease, pneumonia, pulmonary embolism, asthma, chronic rhinosinusitis (including nasal polyps, such as CRSwNP and a non-eosinophilic subtype), chronic obstructive pulmonary disease (COPD), and lung cancer; and cardiovascular disorders including acute cardiac ischemic events, acute myocardial infarction, angina, arrhythmia, atrial fibrulation, atherosclerosis, arterial fibrillation, cardiac insufficiency, cardiovascular disease, chronic heart failure, chronic stable angina, congestive heart failure, coronary artery disease, coronary heart disease, deep vein thrombosis, diabetes, diabetes mellitus, diabetic neuropathy, diastolic dysfunction in subjects with diabetes mellitus, edema, essential hypertension, eventual pulmonary embolism, fatty liver disease, heart disease, heart failure, homozygous familial hypercholesterolemia (HoFH), homozygous familial sitosterolemia, hypercholesterolemia, hyperlipidemia, hypertension, hypertriglyceridemia, metabolic syndrome, mixed dyslipidemia, moderate to mild heart failure, myocardial infarction, obesity management, paroxysmal atrial/arterial fibrillation/fibrulation/flutter, paroxysmal supraventricular tachycardias (PSVT), particularly severe or rapid onset edema, platelet aggregation, primary hypercholesterolemia, primary hyperlipidemia, pulmonary arterial hypertension, pulmonary hypertension, recurrent hemodynamically unstable ventricular tachycardia (VT), recurrent ventricular arrhythmias, recurrent ventricular fibrillation (VF), ruptured aneurysm, sitisterolemia, stroke, supraventricular tachycardia, symptomatic atrial fibrillation/flutter, tachycardia, type-II diabetes, vascular disease, vasculitides (including large vessel vasculitis (LW), medium vessel vasculitis (MW), small vessel vasculitis (SW), variable vessel vasculitis (WV), vingle-organ vasculitis (SOV) and vasculitis associated with systemic disease), venous thromboembolism, ventricular arrhythmias, and other cardiovascular events.

The term “treatment” in relation a given disease or disorder includes, but is not limited to, inhibiting the disease or disorder, for example, arresting the development of the disease or disorder; relieving the disease or disorder, for example, causing regression of the disease or disorder; or relieving a condition caused by or resulting from the disease or disorder, for example, relieving, preventing or treating symptoms of the disease or disorder. The term “prevention” in relation to a given disease or disorder means: preventing the onset of disease development if none had occurred, preventing the disease or disorder from occurring in a subject that may be predisposed to the disorder or disease but has not yet been diagnosed as having the disorder or disease, and/or preventing further disease/disorder development if already present.

In one embodiment, the subject is determined to have a low baseline eosinophil count as compared to a reference level. In one embodiment, the subject is determined to have a low baseline eosinophil count prior to administration of the DGLA.

In one embodiment, the subject is determined to have a normal (e.g., healthy) baseline eosinophil count as compared to a reference level, such as a laboratory reference level. For example, the normal baseline eosinophil count can be less than about 0.2×10⁹/L, less than about 0.25×10⁹/L, less than about 0.3×10⁹/L, less than about 0.4×10⁹/L, less than about 0.5×10⁹/L, less than 0.6×10⁹/L, less than 0.7×10⁹/L or less than about 0.8×10⁹/L. In one embodiment, the subject is determined to have a normal baseline eosinophil count prior to administration of the DGLA or derivative thereof.

The terms “reference level” and “laboratory reference level” can be used interchangeably and include, but are not limited to, a level from a sample collected from a healthy patient. A reference level and/or a laboratory reference level can also be determined from a plurality of samples collected from a population of healthy patients. As one example, a normal eosinophil cell count can be determined based on an eosinophil cell count determined from a population of healthy patients, or a subset of healthy patients, for example, healthy patients of a particular ethnicity. In other embodiments, the reference level and/or laboratory reference level is a value determined from a sample collected at an earlier time point (e.g., 1 day, 3 days, 1 week, 1 month, 3 months, 6 months, 12 months, or more) from the same patient that is undergoing treatment. In some embodiments, the reference level and/or laboratory reference level may be based on values known by those of skill in the art or developed by a medical agency.

In various embodiments, compositions of the invention are administered in an amount sufficient to provide a daily DGLA dose of about 50 mg to about 10000 mg, about 100 mg to about 7500 mg, or about 100 mg to about 5000 mg, for example, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, about 2500 mg about 2600 mg, about 2700 mg, about 2800 mg, about 2900 mg, about 3000 mg, about 3100 mg, about 3200 mg, about 3300 mg, about 3400 mg, about 3500 mg, 3600 mg, about 3700 mg, about 3800 mg, about 3900 mg, about 4000 mg, about 4100 mg, about 4200 mg, about 4300 mg, about 4400 mg, about 4500 mg, 4600 mg, about 4700 mg, about 4800 mg, about 4900 mg, about 5000 mg, about 5100 mg, about 5200 mg, about 5300 mg, about 5400 mg, about 5500 mg of DGLA per day.

In one embodiment, the invention provides a method of treating atopic dermatitis, for example, mild to moderate atopic dermatitis. In one embodiment, the method comprises administering to a subject in need of such treatment DGLA in an amount of about 500 mg to about 3 g per day, about 1 g to about 2.5 g per day, about 1 g per day, or about 2 g per day. In one embodiment, the DGLA is administered to the subject daily for a period of at least about 2 weeks, at least about 4 weeks, or at least about 8 weeks. In a related embodiment, upon treatment in accordance with the present invention, for example, over a period of about 1 to about 12 weeks, about 1 to about 8 weeks, or about 1 to about 4 weeks, the subject or subject group exhibits one or more of the following outcomes:

(a) a reduction in eczema area and severity index (EASI) score relative to baseline or placebo control;

(b) a reduction in percentage of area of an anatomical site affected by atopic dermatitis relative to baseline or control;

(c) a reduction in investigator's global assessment (IGA) score level relative to baseline or placebo control;

(d) a reduction in intensity of erythema, edema/population, oozing/crusts, excoriation, lichenification and/or dryness relative to baseline or placebo control;

(e) a reduction in erythema, edema/population, oozing/crusts, excoriation, lichenification and/or dryness relative to baseline or placebo control;

(f) a reduction in body surface area (BSA) affected by atopic dermatitis relative to baseline or placebo control;

(g) a reduction in loss of sleep relative to baseline or placebo control;

(h) a reduction in occurrence of pruritus (itch) relative to baseline or placebo control;

(i) a reduction in severity of pruritus as an average of the prior three days and/or nights on a visual analog scale (VAS);

(j) a reduction in scoring atopic dermatitis (SCORAD) score relative to baseline or placebo control;

(k) an improved patient-oriented Eczema Measure (POEM) compared to baseline or placebo control;

(l) a reduction in number of days in the preceding week in which the subject reported that the skin was itchy due to eczema;

(m) a reduction in number of days in the preceding week in which the subject reported that their sleep was disturbed due to their eczema;

(n) a reduction in number of days in the preceding week in which the subject experienced skin bleeding;

(o) a reduction in number of days in the preceding week in which the subject experienced skin weeping or oozing clear fluid;

(p) a reduction in number of days in the preceding week in which the subject's skin cracked;

(q) a reduction in number of days in the preceding week in which the subject's skin flaked;

(r) a reduction in number of days in the preceding week in which the subject experienced dry skin;

(s) an increase in trans epidermal water loss compared to baseline or placebo control;

(t) an increase in plasma total and free DGLA compared to baseline;

(u) an increase in DGLA:AA ratio compared to baseline or placebo control;

(v) a reduction in arterial blood pressure compared to baseline or placebo control; and/or

(w) a reduction in dermatology life quality index (DLQI) relative to a baseline or placebo control.

In one embodiment, methods of the present invention comprise measuring baseline levels of one or more markers or parameters set forth in (a)-(w) above prior to dosing the subject or subject group. In another embodiment, the methods comprise administering a composition as disclosed herein to the subject after baseline levels of one or more markers or parameters set forth in (a)-(w) are determined, and subsequently taking an additional measurement of said one or more markers.

In another embodiment, upon treatment with a composition of the present invention, for example, over a period of about 1 to about 12 weeks, about 1 to about 8 weeks, or about 1 to about 4 weeks, the subject or subject group exhibits any 2 or more of, any 3 or more of, any 4 or more of, any 5 or more of, any 6 or more of, any 7 or more of, any 8 or more of, any 9 or more of, any 10 or more of, any 11 or more of, any 12 or more of, any 13 or more of, any 14 or more of, any 15 or more of, any 16 or more of, any 17 or more of, any 18 or more of, any 19 or more of, any 20 or more of, any 21 or more of, or all 22 of outcomes (a)-(w) described immediately above.

In another embodiment, upon treatment with a composition of the present invention, the subject or subject group exhibits one or more of the following outcomes:

(a) a reduction in eczema area and severity index (EASI) score relative to baseline or placebo control of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(b) a reduction in percentage of area of an anatomical site affected by atopic dermatitis relative to baseline or control of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(c) a reduction in investigator's global assessment (IGA) score relative to baseline or placebo control of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(d) a reduction in intensity of erythema, edema/population, oozing/crusts, excoriation, lichenification and/or dryness relative to baseline or placebo control of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(e) a reduction in erythema, edema/population, oozing/crusts, excoriation, lichenification and/or dryness relative to baseline or placebo control of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(f) a reduction in body surface area (BSA) affected by atopic dermatitis relative to baseline or placebo control of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(g) a reduction in loss of sleep relative to baseline or placebo control of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(h) a reduction in occurrence of pruritus (itch) relative to baseline or placebo control of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(i) a reduction in severity of pruritus as an average of the prior three days and/or nights on a visual analog scale (VAS) of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(j) a reduction in scoring atopic dermatitis (SCORAD) score relative to baseline or placebo control of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(k) an improved patient-oriented Eczema Measure (POEM) compared to baseline or placebo control of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(l) a reduction in number of days in the preceding week in which the subject reported that their skin was itchy due to eczema of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(m) a reduction in number of days in the preceding week in which the subject reported that their sleep was disturbed due to their eczema of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(n) a reduction in number of days in the preceding week in which the subject experienced skin bleeding of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(o) a reduction in number of days in the preceding week in which the subject experienced skin weeping or oozing clear fluid of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(p) a reduction in number of days in the preceding week in which the subject's skin cracked of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(q) a reduction in number of days in the preceding week in which the subject's skin flaked of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(r) a reduction in number of days in the preceding week in which the subject experienced dry skin of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(s) an increase in trans epidermal water loss compared to baseline or placebo control of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(t) an increase in plasma total and free DGLA compared to baseline of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(u) an increase in DGLA:AA ratio compared to baseline or placebo control of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(v) a reduction in mean arterial blood pressure of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%; and/or

(w) a reduction in dermatology life quality index (DLQI) relative to a baseline or placebo control, of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%.

In another embodiment, upon treatment with a composition of the present invention after a single dose administration or multiple dose administration, for example over a period of about 1 to about 12 weeks, about 1 to about 8 weeks, or about 1 to about 4 weeks, the subject or subject group exhibits any 2 or more of, any 3 or more of, any 4 or more of, any 5 or more of, any 6 or more of, any 7 or more of, any 8 or more of, any 9 or more of, any 10 or more of, any 11 or more of, any 12 or more of, any 13 or more of, any 14 or more of, any 15 or more of, any 16 or more of, any 17 or more of, any 18 or more of, any 19 or more of, any 20 or more of, any 21 or more of or all 22 of outcomes (a)-(w) described immediately above.

In another embodiment, upon treatment of a subject or subject group (fed or fasted) with a composition comprising about 200 mg of DGLA to about 8000 mg DGLA (administered as one or more dosage units, for example, as 500 mg or 1 g dosage units equating to total daily DGLA doses of about 500 mg, about 1000 mg, about 2000 mg, about 3000 mg, about 4000 mg, about 5000 mg, about 6000 mg, about 7000 mg or about 8000 mg) and after single dose administration or after multiple dose administration, the subject or subject group exhibits one or more of the following outcomes:

(a) a free DGLA Cₘₐₓ (or mean or median Cₘₐₓ) of about 400 ng/ml to about 4500 ng/ml, about 500 ng/ml to about 3400 ng/ml, about 600 ng/ml to about 3300 ng/ml, about 700 ng/ml to about 3200 ng/ml, for example, about 900 ng/ml, about 1000 ng/ml, about 1100 ng/ml, about 1200 ng/ml, about 1300 ng/ml, about 1400 ng/ml, about 1500 ng/ml, about 1600 ng/ml, about 1700 ng/ml, about 1800 ng/ml, about 1900 ng/ml, about 2000 ng/ml, about 2100 ng/ml, about 2200 ng/ml, about 2300 ng/ml, about 2400 ng/ml, about 2500 ng/ml, about 2600 ng/ml, about 2700 ng/ml, about 2800 ng/ml, about 2900 ng/ml, about 3000 ng/ml, about 3100 ng/ml, about 3200 ng/ml, about 3300 ng/ml, about 3400 ng/ml, about 3500 ng/ml, about 3600 ng/ml, about 3700 ng/ml, about 3800 ng/ml, about 3900 ng/ml, about 4000 ng/ml, about 4100 ng/ml, about 4200 ng/ml, about 4300 ng/ml about 4400 ng/ml or about 4500 ng/ml;

(b) a free DGLA Cₘₐₓ/dose (or mean or median Cₘₐₓ/dose) of about 0.5 (1/kL) to about 3 (1/kL), about 0.6 (1/kL) to about 2.5 (1/kL) or about 0.7 (1/kL) to about 2 (1/kL), for example, about 0.7 (1/kL), about 0.8 (1/kL), about 0.9 (1/kL), about 1 (1/kL), about 1.5 (1/kL), about 1.6 (1/kL), about 1.7 (1/kL) or about 1.8 (1/kL);

(c) a free DGLA AUC₀₋₂₄ (or mean or median AUC₀₋₂₄) of about 1500 ng·h/ml to about 12000 ng·h/ml, about 2000 ng·h/ml to about 11000 ng·h/ml or about 2500 ng·h/ml to about 10000 ng·h/ml, for example about 1000 ng·h/ml, about 1500 ng·h/ml, about 2000 ng·h/ml, about 2500 ng·h/ml, about 3000 ng·h/ml, about 3500 ng·h/ml, about 4000 ng·h/ml, about 4500 ng·h/ml, about 5000 ng·h/ml, about 5500 ng·h/ml, about 6000 ng·h/ml, about 6500 ng·h/ml, about 7000 ng·h/ml, about 7500 ng·h/ml, about 8000 ng·h/ml, about 8500 ng·h/ml, about 9000 ng·h/ml, about 9500 ng·h/ml, about 10000 ng·h/ml, about 10500 ng·h/ml, about 11000 ng·h/ml, about 11500 ng·h/ml or about 12000 ng·h/ml.

(d) a free DGLA AUC₀₋₂₄/dose (or mean or median AUC₀₋₂₄/dose) of about 1.5 to about 10 h/kL, about 1.7 to about 8 h/kL or about 2 to about 6 h/kL, for example about 2 h/kL, about 2.5 h/kL, about 3 h/kL, about 3.5 h/kL, about 4 h/kL, about 4.5 h/kL, about 5 h/kL or about 5.5 h/kL;

(e) a free DGLA tₘₐₓ (h) of about 2 to about 10 hours, about 3 to about 8 hours, for example, about 3 hours, about 4 hours, about 5 hours, about 6 hours, about 7 hours or about 8 hours;

(f) a total DGLA Cₘₐₓ (or mean or median total DGLA Cₘₐₓ) of about 4000 ng/ml to about 45000 ng/ml, about 5000 ng/ml to about 34000 ng/ml, about 6000 ng/ml to about 33000 ng/ml, or about 7000 ng/ml to about 32000 ng/ml, for example, about 7000 ng/ml, about 7200 ng/ml, about 7500 ng/ml, about 8000 ng/ml, about 8500 ng/ml, about 9000 ng/ml, about 9500 ng/ml, about 10000 ng/ml, about 11000 ng/ml, about 12000 ng/ml, about 13000 ng/ml, about 14000 ng/ml, about 15000 ng/ml, about 16000 ng/ml, about 17000 ng/ml, about 18000 ng/ml, about 19000 ng/ml, about 20000 ng/ml, about 21000 ng/ml, about 22000 ng/ml, about 23000 ng/ml, about 24000 ng/ml, about 25000 ng/ml, about 26000 ng/ml, about 27000 ng/ml, about 28000 ng/ml, about 29000 ng/ml, about 30000 ng/ml, about 31000 ng/ml, about 32000 ng/ml, about 33000 ng/ml, about 34000 ng/ml, or about 35000 ng/ml;

(g) a total DGLA Cₘₐₓ/dose (or mean or median total DGLA Cₘₐₓ/dose) of about 2 (1/kL) to about 25 (1/kl), about 4 (1/kl) to about 20 (1/kl) or about 5 (1/kl) to about 17 (1/kl), for example about 6 (1/kl), about 9 (1/kl), about 14 (1/kl) or about 16 (1/kl);

(h) a total DGLA AUC₀₋₂₄ (or mean or median total DGLA AUC₀₋₂₄) of about 15000 ng·h/ml to about 900,000 ng·h/ml, about 20,000 ng·h/ml to about 250,000 ng·h/ml or about 25,000 ng·h/ml to about 225,000 ng·h/ml, for example, about 40,000 ng·h/ml, about 210,000 ng·h/ml, about 215,000 ng·h/ml or about 435,000 ng·h/ml;

(i) a total DGLA AUC₀₋₂₄/dose (or mean or median total DGLA AUC₀₋₂₄/dose) of about 50 to about 400 h/kL, about 60 to about 250 h/kL or about 70 to about 225 h/kL, for example, about 80 h/kL, about 100 h/kL, about 110 h/kL or about 215 h/kL;

(j) a total DGLA tₘₐₓ (h) of about 2 to about 25 hours or about 3 to about 20 hours, for example, about 8 hours, about 10 hours, or about 18 hours;

(k) a ratio of total DGLA Cₘₐₓ to free DGLA Cₘₐₓ of about 5:1 to about 12:1, about 6:1 to about 10:1 or about 7:1 to about 9:1, for example, about 7.7:1, about 8.6:1, about 8.8:1 or about 9.8:1;

(l) a steady state free DGLA plasma level (C_{avg}) or mean or median steady state free DGLA plasma level (C_{avg}), after 1 to about 30, 1 to about 28, 1 to about 14 or 1 to about 10 consecutive days of daily administration, of up to about 2000 ng/ml, up to about 750 ng/ml, or up to about 700 ng/ml, for example, about 385 ng/ml or about 675 ng/ml;

(m) a steady state total DGLA plasma level (C_{avg}) or mean or median steady state total DGLA plasma level (C_{avg}), after 1 to about 30, 1 to about 28, 1 to about 14 or 1 to about 10 consecutive days of daily administration, of up to 250,000 ng/ml, up to 180,000 ng/ml, up to 150,000 ng/ml, up to 125,000 ng/ml or up to 100,000 ng/ml; and/or

(n) a ratio of free DGLA plasma to DGLA skin (e.g. as measured in skin blister fluid) from about 0.2:1 to about 5:1, about 0.5:1 to about 2.5:1 or about 0.6:1 to about 1.5:1. In another embodiment, upon treatment with a composition of the present invention, for example, over a period of about 1 to about 12 weeks, about 1 to about 8 weeks, or about 1 to about 4 weeks, the subject or subject group exhibits any 2 or more of, any 3 or more of, any 4 or more of, any 5 or more of, any 6 or more of, any 7 or more of, any 8 or more of, any 9 or more of, any 10 or more of, any 11 or more of, any 12 or more of, any 13 or more of, any 14 or more of, any 15 or more of, any 16 or more of, any 17 or more of, any 18 or more of, any 19 or more of, any 20 or more of, any 21 or more of or all 22 of outcomes (a)-(n) described immediately above.

In another embodiment, upon treatment of fasted and fed subjects or fasted and fed subject groups with a composition comprising about 200 mg of DGLA to about 8000 mg DGLA (administered as one or more dosage units, for example, as 500 mg or 1 g dosage units equating to total daily DGLA doses of about 500 mg, about 1000 mg, about 2000 mg, about 3000 mg, about 4000 mg, about 5000 mg, about 6000 mg, about 7000 mg or about 8000 mg), and after single dose administration or after multiple dose administration, the subject or subject group exhibits one or more of the following outcomes:

(a) a ratio of free DGLA Cₘₐₓ fasted:fed between about 1:1 to about 5:1, for example, of about 2.5:1, of about 3:1 or of about 3.5:1;

(b) a ratio of free DGLA AUC₀₋₂₄ fasted:fed between about 1:1 and about 5:1, for example, of about 1.5:1, of about 2:1, or of about 2.5:1;

(c) a ratio of total DGLA Cₘₐₓ fasted:fed between about 1:1 to about 5:1, for example of about 1:1, of about 1.5:1 or of about 2:1; and/or

(d) a ratio of total DGLA AUC₀₋₂₄ fasted:fed between about 1:1 and about 5:1, for example, of about 1.5:1, of about 2:1 or of about 2.5:1.

In one embodiment, a DGLA-containing composition of the invention comprises the following fatty acid fingerprint:

| | |
|---|---|
| C18:1n-9 | <LOD-<5% |
| C18:2n-6 | <LOD-<5% |
| 20:3ω6-DGLA | NLT 95 |
| isomerA | <LOD-<5% |
| C20:4n-6 + isomerB | <LOD-<5% |
| Total unidentified related | NMT 2 |
| substances | |

In one embodiment, a DGLA-containing composition of the invention comprises the following fatty acid fingerprint:

| | |
|---|---|
| Fatty Acid Profile (Area % FAMEs by GC) | |
| 20:3ω6-DGLA | NLT 95 |
| Related Substances | |
| 20:2ω6 | <LOD-<5% |
| 20:3ω3 | <LOD-<5% |
| 20:4ω6 | <LOD-<5% |
| 20:4ω3 | <LOD-<5% |
| 20:5ω3 | <LOD-<5% |
| Total unidentified related substances | NMT 2 |

An illustrative DGLA-containing composition of the invention comprises the following fatty acid fingerprint:

| | |
|---|---|
| C16:0 | <LOD-<5% |
| C18:1n-7 | <LOD-<5% |
| C18:1n-9 | <LOD-<5% |
| C18:2n-6 | <LOD-<5% |
| C18:3n-6 | <LOD-<5% |
| C20:3n-3 | <LOD-<5% |
| 20:3n-6-DGLA | NLT 95 |
| C20:4n-6 | <LOD-<5% |
| Total unidentified related | NMT 2 |
| substances | |

In one embodiment, a DGLA-containing composition of the invention comprises the following fatty acid fingerprint:

| | |
|---|---|
| Fatty Acid Profile (Area % FAMEs by GC) | NLT 95 |
| 20:3ω6-DGLA | |
| Related Substances | |
| 16:0 | <LOD-<5% |
| 18:3n-6 alcohol methyl ether | <LOD-<5% |
| 18:3n-6 alcohol formate | <LOD-<5% |
| 16:3n-3 | <LOD-<5% |
| 18:1n-9 | <LOD-<5% |
| 18:1n-7 | <LOD-<5% |
| 19:3 | <LOD-<5% |
| 20:1n-9 | <LOD-<5% |
| 20:2n-6 | <LOD-<5% |
| 20:2n-3 + DGLA isomer | <LOD-<5% |
| 20:3n-3 | <LOD-<5% |
| 20:4n-3 | <LOD-<5% |
| Methyl 7,11,14-eicosatrienoate (DGLA | <LOD-<5% |
| isomer) | |
| 22:5n-3 | <LOD-<5% |
| Total unidentified related substances | NMT 2 |

In one embodiment, a DGLA-containing composition of the invention comprises the following fatty acid fingerprint:

| | | |
|---|---|---|
| C20:3 n-6 (DGLA- | Min. | 30% |
| triglycerides) | | |
| C16:0 | Max. | 26% |
| C18:0 | Max. | 12% |
| C18:1 n-9 | Max. | 15% |
| C18:2 n-6 | Max. | 15% |
| C18:3 n-6 | Max. | 5% |
| C20:4 n-6 | Max. | 1% |
| C22:0 | Max. | 5% |
| C24:0 | Max. | 15% |

### EXAMPLES

### Example 1

Three batches of pharmaceutical compositions comprising DGLA (with 2000 pm dl-alpha tocopherol) filled into gelatin capsules were prepared as shown in Table 1.

TABLE 1

| Batch Number | DGLA (mg/Capsule) | Gelatin Description |
|---|---|---|
| E09726/1 | 250 | Standard acid bovine gelatin |
| E09726/2 | 250 | Lime bone gelatin with a lower |
| | | molecular weight (Mw) |
| E09727 | 500 | Standard acid bovine gelatin |

The capsules shells included the following excipients: gelatin, purified water, glycerol, titanium dioxide, and the processing aids lecithin and medium chain triglyceride.

Additional batches of DGLA capsules were also prepared including DGLA FFA (stabilized with a nominal 2000 ppm dl-alpha tocopherol) in capsules containing gelatin, polysorb or mixture of gylycerol/polysorb, purified water, titanium dioxide, and the processing aids lecithin and medium-chain triglyceride as shown in Table 2.

TABLE 2

| Batch | DGLA | | |
|---|---|---|---|
| Number | (mg/Capsule) | Gelatin Description | Plasticizer |
| E09778 | 500 | Lime bone gelatin with a | Glycerol |
| | | lower molecular weight | |
| | | (Mw) | |
| E09777/01 | 500 | Lime bone gelatin with a | Polysorb (D-sorbitol |
| | | lower molecular weight | and 1,4-sorbitan |
| | | (Mw) | sugar alcohols in |
| | | | water solution) |
| E09777/02 | 500 | Lime bone gelatin with a | Gycerol + Polysorb |
| | | lower molecular weight | |
| | | (Mw) | |
| E09777/03 | 500 | Lime bone gelatin with a | Polysorb |
| | | lower molecular weight | |
| | | (Mw)—Even Lower Mw | |
| | | (Advanced RXL Gelatine) | |

Capsule shell compositions for each of the batches are shown below in Tables 3 and 4.

TABLE 3

| | Unit Quantity | | | |
|---|---|---|---|---|
| | mg/capsule | | | |
| Active Substance | 500 mg | | | |
| DGLA | E09778 | E09777/01 | E09777/02 | E09777/03 |
| Wet Gelatin Shell Mass | | | | |
| Gelatin | 128.97 | 128.97 | 128.97 | 128.97 |
| | (RXL) | (RXL) | (RXL) | (RXL Adv.)¹ |
| Total Glycerol | 67.70 | 2.94 | 35.32 | 2.94 |
| Polysorb | N/A | 64.76 | 32.38 | 64.76 |
| Purified Water | 100.38 | 100.38 | 100.38 | 100.38 |
| Titanium Dioxide | 2.94 | 2.94 | 2.94 | 2.94 |
| Lecithin | Trace | Trace | Trace | Trace |
| Triglycerides Medium | Trace | Trace | Trace | Trace |
| Chain | | | | |

¹RXL gelatin contains a lower number of high molecular weight polymers (~5% >200,000 Da)

TABLE 4

| | Unit Quantity | | | |
|---|---|---|---|---|
| E09726/01 | mg/capsule | % w/w | mg/capsule | % w/w |
| Active Substance | | | | |
| DGLA | 500 | 100 | 250 | 100 |
| Wet Gelatin Shell | | | | |
| Mass² | | | | |
| Gelatin (not RXL) | 132.35 | 44.12 | 87.79 | 44.12 |
| Total Glycerol | 76.76 | 25.59 | 50.92 | 25.59 |
| Purified Water | 87.95 | 29.31 | 58.33 | 29.31 |
| Glycerol and | — | — | — | — |
| Polysorb? | | | | |
| Titanium Dioxide | 2.94 | 0.98 | 1.95 | 0.98 |
| Lecithin | Trace | Trace | Trace | Trace |
| Triglycerides Medium | Trace | Trace | Trace | Trace |
| Chain | | | | |

Stability testing of the above capsules was performed. Capsules from each batch were maintained for up to 6 months and assessed using a qualitative or quantitative USP 2040 Disintegration and Dissolution test protocol. Results are shown in Tables 5-7.

TABLE 5
Stability Data for DGLA Softgel capsules: Qualitative Rupture Test Results

| Specification: | Results (Months): | | | | | | | |
|---|---|---|---|---|---|---|---|---|
| read | | | | 3 | | | | |
| and | | | | 25° C./ | | 30° C./ | | 40° C./ |
| record | | 1 | 2 | 60% RH | | 65% RH | | 75% RH |
| (min) | 0 | 40° C./ | 40° C./ | Without | With | Without | With | Without |
| Batches | N/A | 75% RH | 75% RH | pepsin | pepsin | pepsin | pepsin | pepsin |
| E09726/ | 7 | 6 | >30/ | 8 | N/A | 1 | NP | >30 |
| 01 | | | 22⁽¹⁾ | | | caps | | |
| | | | | | | on 6 | | |
| | | | | | | >30 | | |
| E09726/ | 4 | 4 | 4 | 3 | N/A | | | 1 |
| 02 | | | | | | | | caps |
| | | | | | | | | on 6 |
| | | | | | | | | >30 |
| E09727 | | | >30 | 4 | 10 | >30 | NP | >30 |
| | | | | caps | | | | |
| | | | | on 6 | | | | |
| | | | | >30 | | | | |

| Specification: | Results (Months): | | | | | | |
|---|---|---|---|---|---|---|---|
| read | | 6 | | | | | |
| and | | 25° C./ | | 30° C./ | | 40° C./ | |
| record | 3 | 60% RH | | 65% RH | | 75% RH | |
| (min) | With | Without | With | Without | With | Without | With |
| Batches | pepsin | pepsin | pepsin | pepsin | pepsin | pepsin | pepsin |
| E09726/ | 14 | >30 | | >30 | | >30 | 2 |
| 01 | | | | | | | caps |
| | | | | | | | on |
| | | | | | | | 6 >30 |
| E09726/ | 6 | 1 | 10 | 2 | 9 | 12 | 15 |
| 02 | | caps | | caps | | | |
| | | on 6 | | on 6 | | | |
| | | >30 | | >30 | | | |
| E09727 | 15 | >30 | 14 | >30 | 16 | >30 | 3 |
| | | | | | | | caps |
| | | | | | | | on |
| | | | | | | | 6 >30 |

TABLE 6
DGLA glyceride percentages

| Batch | Time | 25 | 30 | 40 |
|---|---|---|---|---|
| Time point (months) | | DGLA glycerides % | | |
| E09727 | 0 | 0 | 0 | 0 |
| | 1 | 0 | 0 | 0.54 |
| | 2 | 0 | 0 | 1.19 |
| | 3 | 0.41 | 0.67 | 2.1 |
| | 6 | 0.53 | 1.43 | 5.81 |
| | 0 | 0 | 0 | 0 |
| | 1 | 0 | 0 | 0.56 |
| E09726/1 | 2 | 0 | 0 | 1.35 |
| | 3 | 0.37 | 0.58 | 2.31 |
| | 6 | 0.68 | 1.42 | 6.8 |
| | 0 | 0 | ND | 0 |
| | 1 | 0 | ND | ND |
| E09726/2 | 2 | 0 | ND | 1.63 |
| | 3 | 0.51 | ND | ND |
| | 6 | 1.16 | ND | 8.55 |

TABLE 7

| | | | | Mono, Di and | | | |
|---|---|---|---|---|---|---|---|
| | | | | Triglycerides | DGLA | Quantitative | |
| | | Storage | Acid | assay (% DGLA) | esters (%) | rupture test | |
| | | Conditions | value | Read and | Read and | Read and record | |
| Specifications | | 40° C./75% RH | 176-184 | record | record | (mean %) | |
| E09778 | (RXL + | 1 month | 178 | 0.98 | 1.8 to 2.6² | | Tier I |
| | Glycerol) | | | | | 15 min | 94 |
| | | | | | | 30 min | 102 |
| | | | | | | 45 min | 102 |
| | | | | | | 60 min | 102 |
| | | 3 months | 172 | 3.08 | N/A | | Tier II |
| | | | | | | | stage 2 |
| | | | | | | 15 min | N/A |
| | | | | | | 30 min | 92 |
| | | | | | | 45 min | 96 |
| | | | | | | 60 min | 97 |
| E09777/01 | (RXL + | 1 month | 180 | N/A | 1.8 to 2.9² | | Tier I |
| | Polysorb) | | | | | 15 min | 81 |
| | | | | | | 30 min | 91 |
| | | | | | | 45 min | 96 |
| | | | | | | 60 min | 102 |
| | | 3 months | 182 | N/A | Not | | Tier II |
| | | | | | available | 15 min | N/A |
| | | | | | | 30 min | 97 |
| | | | | | | 45 min | 99 |
| | | | | | | 60 min | 98 |
| E09777/02 | (RXL + | 1 month | 177 | <0.5 | 0.0 to 3.4² | | Tier I |
| | Glycerol + | | | | | 15 min | 95 |
| | Polysorb) | | | | | 30 min | 101 |
| | | | | | | 45 min | 101 |
| | | | | | | 60 min | 102 |
| | | 3 months | 177 | 3.1 | Not | | Tier I |
| | | | | | avalable | 15 min | 92 |
| | | | | | | 30 min | 97 |
| | | | | | | 45 min | 97 |
| | | | | | | 60 min | 98 |
| E09777/03 | (RXL adv. + | 1 month | 171⁽²⁾ | N/A | 2.7 to 3.6² | | Tier I |
| | Polysorb) | | | | | 15 min | 88 |
| | | | | | | 30 min | 97 |
| | | | | | | 45 min | 99 |
| | | | | | | 60 min | 99 |
| | | 3 months | 174 | N/A | Not | | Tier I |
| | | | | | Available | 15 min | 87 |
| | | | | | | 30 min | 94 |
| | | | | | | 45 min | 95 |
| | | | | | | 60 min | 95 |

As seen above, there was a slowdown in dissolution rate in water over time for capsules formulated with glycerol and standard acid bovine gelatin (E09726/01, and E09727). There was a DGLA release rate of greater than 30 mins after 6 months at 40° C./75% RH in simulated gastric fluid (pH 1.2, pepsin).

A DGLA release rate of less than 30 mins after 6 months at 40° C./75% RH was only achieved in simulated gastric fluid (pH 1.2, pepsin) with capsules containing lime bone gelatin with a lower molecular weight (Mw) (E09777/02).

There was a significant increase in DGLA glyceride formation over time in DGLA capsule shells containing glycerol (Table 4). This was temperature dependent with highest concentrations of DGLA formed at 40° C. 75% RH.

Polysorb is commonly used as a hydrophilic plasticizer to limit exchange between capsule fill media and shell. D-Sorbitol and 1,4-sorbitan have a higher MW than glycerol which limits its mobility through the gelatin shell. Despite this, there was still interaction of D-Sorbitol and 1,4-sorbitan to form DGLA FFA esters in batches E09777 1/2 and 3.

There was no reduction in acid value of the DGLA for batches formulated with D-Sorbitol and 1,4-sorbitan (E09777 1/02/3) whereas there was a reduction in acid value for E09778 formulated with glycerol.

There was no slowdown in dissolution rate in water over time for capsules formulated with D-Sorbitol and 1,4-sorbitan (E09777/03). The DGLA release rate was less than 30 minutes in water after 3 months 40° C./75% RH.

### Example 2

A randomized, double-blind, placebo-controlled, phase II study was performed to assess the efficacy and safety of orally administered DS107G (DGLA) to patients with moderate to severe atopic dermatitis. The primary objective of the study was to compare the efficacy of orally administered DS107G capsules, versus placebo, in the treatment of adult patients with moderate to severe atopic dermatitis. The secondary objective was to assess the safety of orally administered DS107G capsules, versus placebo, in adult patients with moderate to severe atopic dermatitis. The primary study endpoint was the proportion of patients achieving an IGA (Investigator Global Assessment) of 0 (clear) or 1 (almost clear) and a decrease of at least 2 points in IGA at week 8. Secondary study endpoints included:

Change from baseline in IGA at week 2, 4 and 8;

Change from baseline in EASI (Eczema Area and Severity Index) at week 2, 4 and 8;

Proportion of patients achieving at least a 1-point decrease in IGA at week 8;

Change from baseline in the Patient Orientated Eczema Measure (POEM) at week 2, 4 and 8;

Change from baseline in the Dermatology Life Quality Index (DLQI) score at week 2, 4 and 8;

Change from baseline in SCORAD at week 2, 4 and 8;

Change from baseline in the patient's visual analog scale pruritus score at week 2, 4 and 8;

Change from baseline in Body Surface Area (BSA) at week 2, 4 and 8; and/or

Number of treatment-emergent adverse events (TEAEs) in each treatment group.

Exploratory Endpoints included:

Change from baseline in Trans epidermal water loss (TEWL) at week 2, 4 and 8 (selected sites only);

Plasma total and free DGLA concentrations at Baseline, weeks 4 and 8;

Plasma total fatty acid profile at Baseline, week 4 and week 8 (sample to be retained and analyzed at a later date); and/or

Interleukin profile at Baseline, week 4 and week 8 (Sample to be retained and analyzed at a later date).

Approximately 100 patients with moderate to severe atopic dermatitis were included in this multicenter, double-blind, placebo controlled, phase IIa study. All subjects signed an informed consent and underwent screening for study eligibility. Subjects were randomized (1:1) at baseline visit to either receive oral DS107G 2 g or placebo, once daily for 8 weeks in a fasting state.

Subjects visited the clinic on 6 occasions: at screening, baseline, week 2, week 4, week 8 (end of treatment/early termination) and week 10 (follow-up). All subjects exited the study at the week 10 visit. The primary efficacy variable was the proportion of patients achieving an IGA of 0 (clear) or 1 (almost clear), and a decrease of at least 2 points in IGA at week 8. Secondary efficacy variables included: IGA at other visits, pruritus (obtained from the SCORAD visual analog scale), EASI, BSA, POEM, DLQI, SCORAD and TEWL (for selected sites only). Safety was assessed through adverse events, physical examination, vital signs and safety laboratory tests (including pregnancy tests for women of childbearing potential). Pharmacokinetic samples were obtained at Baseline (Day 0), week 4 and week 8 visits in order to measure total and free DGLA plasma trough levels. Separate plasma samples were retained for later analysis of total fatty acid profile and interleukin profile.

The EASI is a composite score ranging from 0-72 that takes into account the degree of erythema, induration/papulation, excoriation, and lichenification (each scored from 0 to 3 separately) for each of four body regions, with adjustment for the percent of BSA involved for each body region and for the proportion of the body region to the whole body. A detailed procedure of EASI score calculation is provided below.

Four anatomic sites—head, upper extremities, trunk and lower extremities—are assessed for erythema, induration (papules), excoriation and lichenification as seen on the day of the examination. The severity of each sign is assessed using a 4-point scale:
0=No symptoms
1=Slight
2=Moderate
3=Marked

The area affected by atopic dermatitis within a given anatomic site is estimated as a percentage of the total area of that anatomic site and assigned a numerical value according to the degree of atopic dermatitis involvement as follows:
0=no involvement
1=<10%
2=10 to <30%
3=30 to <50%
4=50 to <70%
5=70 to <90%
6=90 to 100%

The EASI score is obtained by using the formula

EASI=0.1(Eₕ+Iₕ+Exₕ+Lₕ)Aₕ+0.2(Eᵤ+Iᵤ+Exᵤ+Exᵤ)Aᵤ+0.3(Eₜ+Iₜ+Exₜ+Exₜ)At+0.4(Eₗ+Iₗ+Exₗ+Exₗ)Aₗ

Where E, I, Ex, L and A denote erythema, induration, excoriation, lichenification and area, respectively, and h, u, t, and l denote head, upper extremities, trunk, and lower extremities, respectively.

SCORAD is calculated as follows: Six items (erythema, edema/papulation, oozing/crusts, excoriation, lichenification, and dryness) are selected to evaluate the AD severity. The intensity of each item is graded using a 4-point scale:
0=No symptoms
1=Mild
2=Moderate
3=Severe

The area chosen for grading must be representative (average intensity) for each item. The individual intensity ratings for each item will then be added (ranging from 0-18) and multiplied by 3.5, giving a maximal score of 63.

The overall BSA affected by AD is evaluated (from 0 to 100%) and divided by 5. One patient's palm represents 1% of his/her total BSA. The maximum is 20.

Subjective items include loss of sleep and the occurrence of pruritus. These are evaluated by asking patients to indicate on the 10-cm scale (0-10) on the assessment form the point corresponding to the average value for the last three days/nights. The combined maximum score of these two is 20.

The sum of the above measures represents the SCORAD which can vary from 0 to 103. If the subjective scores of pruritus and loss of sleep are excluded, the SCORAD becomes objective SCORAD (score range 0-83).

Investigator's global assessment is determined as follows:

| Score | Grade | Definition |
|---|---|---|
| 0 | Clear | No evidence of disease with the exception of |
| | | residual pigment changes and/or xerosis |
| 1 | Almost clear | Perceptible erythema, papulation/infiltration |
| 2 | Mild | Mild erythema, papulation/infiltration |
| 3 | Moderate | Moderate erythema, papulation/infiltration |
| 4 | Severe | Severe erythema, papulation/infiltration |
| 5 | Very Severe | Severe erythema, papulation/infiltration with |
| | | oozing/crusting |

Body Surface Area (BSA). The overall BSA affected by AD will be evaluated (from 0 to 100%) at each visit. One patient's palm represents 1% of his/her total BSA. For all study visits, except at screening, the BSA of involved skin will be measured with the SCORAD measurement and evaluated as a separate endpoint.

The Patient-Oriented Eczema Measure (POEM) is determined using the following questionnaire:

| | |
|---|---|
| Patient ID #: __ __ - __ __ __ | Patient Initials: ___ ___ ___ |
| Visit Day: _____________ | Visit Date (dd-mmm-yyyy): ________ |

Please circle one response for each of the seven questions below about
your eczema, Please leave blank any questions you feel unable to
answer.

1. Over the last week, on how many days has your skin been itchy
because of your eczema?

| | | | | |
|---|---|---|---|---|
| No days | 1-2 days | 3-4 days | 5-6 days | Every day |

2. Over the last week, on how many nights has your sleep been disturbed
because of your eczema?

| | | | | |
|---|---|---|---|---|
| No days | 1-2 days | 3-4 days | 5-6 days | Every day |

3. Over the last week, on how many days has your skin been bleeding
because of your eczema?

| | | | | |
|---|---|---|---|---|
| No days | 1-2 days | 3-4 days | 5-6 days | Every day |

4. Over the last week, on how many days has your skin been weeping or
oozing clear fluid because of your eczema?

| | | | | |
|---|---|---|---|---|
| No days | 1-2 days | 3-4 days | 5-6 days | Every day |

5. Over the last week, on how many days has your skin been cracked
because of your eczema?

| | | | | |
|---|---|---|---|---|
| No days | 1-2 days | 3-4 days | 5-6 days | Every day |

6. Over the last week, on how many days has your skin been flaking off
because of your eczema?

| | | | | |
|---|---|---|---|---|
| No days | 1-2 days | 3-4 days | 5-6 days | Every day |

7. Over the last week, on how many days has your skin felt dry or rough
because of your eczema?

| | | | | |
|---|---|---|---|---|
| No days | 1-2 days | 3-4 days | 5-6 days | Every day |

The Dermatology Life Quality Index (DLQI) is determined using the following questionnaire:

The aim of this questionnaire is to measure how much your skin
problem has affected your life OVER THE LAST WEEK, Please
check one box for each question.

| | | | | |
|---|---|---|---|---|
| 1. | Over the last week, how itchy, sore, | Very | □ | |
| | painful or stinging has your skin | much | □ | |
| | been? | A lot | □ | |
| | | A little | □ | |
| | | Not at all | | |
| | Over the last week, how embarrassed | Very | □ | |
| | or self conscious have you been | much | □ | |
| | because of your skin? | A lot | □ | |
| | | A little | □ | |
| | | Not at all | | |
| 3. | Over the last week, how much has | Very | □ | |
| | your skin interfered with you going | much | □ | |
| | shopping or looking after your home | A lot | □ | |
| | or yard? | A little | □ | Not relevant |
| | | Not at all | | □ |
| 4. | Over the last week, how much has | Very | □ | |
| | your skin influenced the clothes you | much | □ | |
| | wear? | A lot | □ | |
| | | A little | □ | Not relevant |
| | | Not at all | | □ |
| 5. | Over the last week, how much has | Very | □ | |
| | your skin affected any social or | much | □ | |
| | leisure activities? | A lot | □ | |
| | | A little | □ | Not relevant |
| | | Not at all | | □ |
| 6. | Over the last week, how much has | Very | □ | |
| | your skin made it difficult for you to | much | □ | |
| | do any sport? | A lot | □ | |
| | | A little | □ | Not relevant |
| | | Not at all | | □ |
| 7. | Over the last week, has your skin | yes | □ | |
| | prevented you from working or | no | □ | Not relevent |
| | studying? | | | □ |
| | If “No”, over the last week how | A lot | □ | |
| | much has your skin been a problem | A little | □ | |
| | at work or studying? | Not at all | □ | |
| 8. | Over the last week, how much has | Very | □ | |
| | your skin created problems with | much | □ | |
| | your partner or any of your close | A lot | □ | |
| | friends or relatives? | A little | □ | Not relevant |
| | | Not at all | | □ |
| 9. | Over the last week, how much has | Very | □ | |
| | your skin caused any sexual | much | □ | |
| | difficulties? | A lot | □ | |
| | | A little | □ | Not relevant |
| | | Not at all | | □ |
| 10 | Over the last week, how much of a | Very | □ | |
| | problem has the treatment for your | much | □ | |
| | skin been, for example by making | A lot | □ | |
| | your home messy, or by taking up | A little | □ | Not relevant |
| | time? | Not at all | | □ |

©AY Finlay, GK Khan, April 1992 www.dermatology.org.uk.
Please check you have answered EVERY question. Thank you.

Inclusion criteria were as follows:
1. Male or female subject aged 18 years and older on the day of signing the informed consent form (ICF).
2. Clinically confirmed diagnosis of active atopic dermatitis according to Hanifin and Rajka criteria (Appendix G).
3. Moderate to severe atopic dermatitis at baseline as defined by an IGA of minimum 3 at baseline visit.
4. Atopic dermatitis covering minimum 10% of the body surface area at baseline.
5. Body mass index (BMI) is between 18 and 35 kg/m² inclusively.
6. Female patients of childbearing potential must use adequate contraception or have a sterilized partner for the duration of the study: systemic hormonal contraceptives, intrauterine device or barrier method of contraception in conjunction with spermicide, or agree to sexual abstinence. Hormonal contraceptives must be on a stable dose for at least one month before baseline. Note: Women of non-child bearing potential are:
   women who have had surgical sterilization (hysterectomy or bilateral oophorectomy or tubal ligation)
   women greater than 60 years of age
   women greater than 40 and less than 60 years of age who have had a cessation of menses for at least 12 months and a follicle-stimulating hormone (FSH) test confirming non-childbearing potential (FSH ≥40 mIU/mL) or cessation of menses for at least 24 months without FSH levels confirmed.
7. Patients who are able and willing to stop treatment for atopic dermatitis throughout the study (except for allowed emollients).
8. Capable and willing to give signed informed consent and the consent must be obtained prior to any study related procedures.

Exclusion criteria were as follows:
1. Female patients with positive pregnancy test at screening or Day 0 visit (baseline) or lactating women.
2. Any clinically significant controlled or uncontrolled medical condition or laboratory abnormality that would, in the opinion of the investigator, put the patient at undue risk or interfere with interpretation of study results.
3. Clinically significant impairment of renal or hepatic function.
4. Other skin conditions that might interfere with atopic dermatitis diagnosis and/or evaluation (such as psoriasis or current viral, bacterial and fungal skin infections).
5. History of hypersensitivity to any substance in DS107G or placebo capsules.
6. Use of biologics 3 months prior to start of treatment/Day 0 visit (baseline), or 5 half-lives (whichever is longer).
7. Use of systemic treatments (other than biologics) that could affect atopic dermatitis less than 4 weeks prior to baseline visit (Day 0), e.g., retinoids, calcineurin inhibitors, methotrexate, cyclosporine, hydroxycarbamide (hydroxyurea), azathioprine and oral/injectable corticosteroids. Intranasal corticosteroids and inhaled corticosteroids for stable medical conditions are allowed.
8. Treatment with any experimental drug within 30 days prior to Day 0 visit (baseline), or 5 half-lives (whichever is longer).
9. Excessive sun exposure, use of tanning booths or other ultraviolet (UV) light sources 4 weeks prior to Day 0 visit (baseline) and/or is planning a trip to sunny climate or to use tanning booths or other UV sources between screening and follow-up visits.
10. Use of any topical medicated treatment for atopic dermatitis 2 weeks prior to start of treatment/Day 0 visit (baseline), including but not limited to, topical corticosteroids, calcineurin inhibitors, tars, bleach, antimicrobials and bleach baths.
11. Use of topical products containing urea, ceramides or hyaluronic acid 2 weeks prior to Day 0.
12. Use of anti-histamines for atopic dermatitis within 2 weeks of baseline.
13. Significant uncontrolled cardiovascular (a history of ECG abnormalities that are clinically significant in the opinion of the investigator), neurologic, malignant, psychiatric, respiratory or hypertensive disease, as well as diabetes and arthritis.
14. Medical history of chronic infectious disease (e.g., hepatitis B, hepatitis C or infection with human immunodeficiency virus).
15. History of clinically significant drug or alcohol abuse in the last year prior to Day 0 (baseline).

Continuous variables are summarized in tables and included the number of subjects, mean, standard deviation, median, minimum, maximum and inter-quartile range. Categorical variables are presented in tables as frequencies and percentages. All statistical tests were two-sided and were performed with a significant level of 0.05, unless otherwise specified.

Each subject was questioned on the specific points listed below prior to drug administration. If a subject admitted a non-compliance with these restrictions, the Principal Investigator (or designee) and/or the sponsor determined whether or not the subject was permitted to remain in the study. Non-compliance with these restrictions was recorded.

Subjects were instructed to abstain from planning a trip to sunny climate or use of tanning equipment between screening and follow-up visits.

Subjects were instructed to abstain from using any drugs/treatments that may influence atopic dermatitis (refer to exclusion criteria and prohibited therapies or procedures section) throughout the study.

Subjects were required to start fasting at least 8 hours before drug administration upon waking. Fasting continued for at least 60 minutes following drug administration, after which subject could have breakfast. Water was allowed at all times during the fasting period, but no other fluids were permitted. Medication(s) for other conditions that are permitted in the study could be taken as usual.

For Baseline (Day 0), week 4 and week 8 visits, a blood draw was performed for PK analysis. PK samples were taken pre-dose; therefore, study drug administration occurred during the visit for Day 0 and week 4 visits. Because dosing occurred at the clinic on Day 0 and week 4, subjects were required to fast for at least 8 hours prior to study drug administration and were allowed to have a meal 60 minutes after study drug administration.

Subjects had the right to withdraw from the study at any time for any reason without penalty. The investigator also had the right to withdraw subjects from the study in the best interest of the subject or if the subject was uncooperative or non-compliant. The investigator or one of his or her staff members contacted the subject either by telephone or through a personal visit to determine as completely as possible the reason for the withdrawal, and record the reason in subject's source document and CRF. A complete final early termination (week 8) evaluation at the time of the subject's withdrawal was made with an explanation of why the subject was withdrawing from the study. If the reason for removal of a subject was an adverse event or an abnormal laboratory test result, the principal specific event or test was recorded. Subjects who discontinued the study before the week 8 visit were asked to come for an early termination visit as soon as possible, and have the assessments listed at week 8 performed. They were also asked to return two weeks later for the safety assessments listed at week 10.

Subjects who fulfilled all the inclusion and none of the exclusion criteria were accepted in the study. Each subject read and signed an informed consent form prior to any screening procedures being performed. This study involved a comparison of DS107G (DGLA) with placebo, administered orally once daily upon waking for a total duration of 8 weeks. Subjects were randomized to one of the two treatment groups in a 1:1 ratio:

Treatment group A: 2 grams DS107G (4 capsules)

Treatment group B: 2 grams placebo capsules (4 capsules)

Subjects were required to start fasting at least 8 hours before drug administration upon waking. Fasting continued for at least 60 minutes following drug administration, after which, subjects were allowed to have breakfast. Water was allowed at all times during the fasting period, but no other fluids were permitted. Medication(s) for other conditions that are permitted in the study could be taken as usual.

DS107G capsules were provided by Dignity Sciences as opaque, oval soft gelatin capsules containing 500 mg of DGLA free fatty acid (FFA). Placebo capsules were also provided by Dignity Sciences as opaque, oval soft gelatin capsules containing 500 mg of liquid paraffin. DS107G capsules were supplied in manufactured form (blinded), and packaged in aluminum foil blisters of 28 units. Placebo was presented in identical blisters and packs and stored/packaged the same as DS107G capsules. Study medication was labelled according to US and Canadian regulations.

The study medication was provided by the sponsor to the investigator and was kept, on site, in a locked room or cabinet with limited access. DS107G and placebo capsules were stored at a controlled room temperature between 15-30° C. and were only supplied to subjects in the trial under the supervision of the investigator. Study drug was dispensed by the study site to the subject at each study visit. Subjects were to return all study drug blister packs (used and unused) to the study site. The capsules within blister packs were counted prior to dispensing and upon return and the counts were recorded in the source documents and eCRF. Each subject was instructed on the importance of returning study drug at the next study visit. If a subject did not return study drug, he/she was instructed to return it as soon as possible.

All medications (including over-the-counter drugs, vitamins, and antacids) taken ≤4 weeks prior to screening and throughout the study were recorded. All medications taken for atopic dermatitis in the 2 months prior to screening were recorded. Subjects were allowed to apply a bland emollient of their choice on their skin, including AD lesions, provided that emollient use was initiated at least 2 weeks prior to Day 0 and continued at the same frequency and on the same skin areas throughout the study. Subjects were requested to avoid using emollients containing any active ingredient which has or may have an effect on atopic dermatitis including the following ingredients: urea, ceramide or hyaluronic acid.

Non-sedative anti-histamines (e.g. loratadine, fexofenadine) were allowed during the study only if used to treat medical conditions other than atopic dermatitis. Such medications were allowed during the study only if the subject was on a stable dose for at least 2 weeks prior to Day 0 and continued to use the same agent everyday throughout the study. Inhaled and intranasal corticosteroids for stable medical conditions were allowed.

The following topical therapies or procedures were prohibited during the study for all subjects:

Topical medicated treatments that could affect atopic dermatitis, including but not limited to: topical corticosteroids, calcineurin inhibitors, tars, bleach, antimicrobials, bleach baths;

Any topical product containing urea, ceramides or hyaluronic acid;

Systemic therapy that could affect atopic dermatitis, e.g. retinoids, calcineurin inhibitors, methotrexate, cyclosporine, hydroxycarbamide (hydroxyurea), azathioprine and oral/injectable corticosteroids;

Anti-histamines (except non-sedative anti-histamine);

Any biological agent;

UVA or UVB phototherapy;

Psoralen+Ultraviolet A (PUVA) therapy;

Excessive sun exposure or use of tanning booth; and/or

Any investigational agent.

Treatment compliance was assessed at each visit by direct questioning, review of the subject's compliance log and capsule count, and was based on the latter. Subjects were given a paper diary at each visit along with study medication. Subjects indicated any missed doses in the diary, as well as the timing of the last food ingestion prior to study drug administration and food ingestion following study drug administration. Subjects were instructed to bring all capsules and blister packs (used and unused) and compliance log to the next study visit. Any deviation from the prescribed dosage regimen was recorded in the source document and in the eCRF. Subjects who were significantly noncompliant were counseled.

Clinical evaluations of atopic dermatitis were performed by an experienced and qualified dermatologist (board certified or equivalent). To assure consistency and reduce variability, the same assessor performed all assessments on a given subject whenever possible.

The Investigator's Global Assessment (IGA) of Disease Severity was assessed at each visit. The IGA is a global assessment of the current state of the disease. It is a 6-point morphological assessment of overall disease severity and will be determined according to the following definitions: 0 (clear), 1 (almost clear), 2 (mild), 3 (moderate), 4 (severe) and 5 (very severe). In order to be eligible, subjects must have an IGA score >/=3 at Baseline visit (Day 0).

The Eczema Area and Severity Index (EASI) was assessed at each visit, except screening visit. It quantifies the severity of a subject's atopic dermatitis based on both lesion severity and the percent of BSA affected. The EASI is a composite score ranging from 0-72 that takes into account the degree of erythema, induration/papulation, excoriation, and lichenification (each scored from 0 to 3 separately) for each of four body regions, with adjustment for the percent of BSA involved for each body region and for the proportion of the body region to the whole body.

The overall BSA affected by AD was evaluated (from 0 to 100%) at each visit. One patient's palm represents 1% of his/her total BSA. For all study visits except at screening, the BSA of involved skin will be measured with the SCORAD measurement (see below for description) and evaluated as a separate endpoint. In order to be eligible, subjects must have a BSA of at least 10% at Baseline visit (Day 0).

SCORAD was measured at each visit, except the screening visit. The SCORAD grading system was developed by the European Task Force on Atopic Dermatitis (1993) and has been a standard tool to assess the AD severity in clinical studies in Europe. Six items (erythema, edema/papulation, oozing/crusts, excoriation, lichenification, and dryness) were selected to evaluate the AD severity. The overall BSA affected by AD was evaluated (from 0 to 100%) and included in the SCORAD scores. Loss of sleep and pruritus were evaluated by patients on a visual analog scale (0-10). The sum of these measures represents the SCORAD, which can vary from 0 to 103.

For all study visits except screening, the pruritus severity score was recorded with the SCORAD measurement and was evaluated as a separate endpoint. This was evaluated by asking subjects to indicate on the 10-cm scale (0-10) of the assessment form the point corresponding to the average value for the last three days/nights.

TEWL. The clinical severity of AD and associated effect on skin barrier function will be evaluated at each visit, except the screening visit. This evaluation will be performed at selected sites that have demonstrated previous experience with this device.

At Baseline (Day 0), the investigator will select three representative areas of active AD for each subject; the location of these sites will be recorded. At subsequent visits, TEWL readings for each area of AD will be taken in standard room ambient conditions (22-25° C., 40-60% relative humidity); the mean of the TEWL measurements will be used for the analyses.

The Patient-Oriented Eczema Measure (POEM) was assessed at each visit, except screening visit. The POEM is a self-assessment of disease severity by the patient. POEM has a maximum value of twenty eight based on the patient's response to seven questions scored according to the following scale:
No Days=0
1-2 Days=1
3-4 Days=2
5-6 Days=3
Everyday=4

The DLQI is a simple 10-question validated questionnaire which was completed at each visit, except screening.

The clinical severity of AD and associated effect on skin barrier function was evaluated at each visit, except the screening visit. This evaluation was performed at selected sites that have demonstrated previous experience with this device.

At Baseline (Day 0), the investigator selected three representative areas of active AD for each subject; the location of these sites was recorded. At subsequent visits, TEWL readings for each area of AD were taken in standard room ambient conditions (22-25° C., 40-60% relative humidity); the mean of the TEWL measurements were used for the analyses.

Laboratory tests were performed at screening, Day 0, Week 4 and Week 8. If Week 8 results indicated a clinically significant change from baseline, laboratory tests were also performed at Week 10. The tests included urinalysis, hematology with differential and coagulation testing, a standard chemistry panel (chemistry includes liver function tests and cholesterol), coagulation, serum pregnancy test (screening and week 8 early termination visits) for women of childbearing potential (WOCBP). At baseline (Day 0), week 2, week 4 and week 10 visits, a urine pregnancy test was performed for women of childbearing potential (conducted at the investigator site). At the screening visit, FSH levels were tested for women greater than 40 and less than 60 years of age who have had a cessation of menses for at least 12 months but less than 24 months.

At Baseline, week 4 and week 8 visits, blood draws were performed prior to study drug administration (no study drug administration at week 8 visit). If a subject came to the clinic after taking their daily dose of study medication, this subject was required to come back the following day for PK blood draws. Total DGLA and free DGLA trough plasma levels were measured. A second blood draw was performed for later evaluation of total fatty acid profile in plasma. The blood draw for serum chemistry analysis was split in two aliquots for chemistry analysis and later evaluation of interleukins.

The primary endpoint can be translated as a responder analysis where a subject will be classified as Responder if he/she achieves an IGA score of 0 (clear) or 1 (almost clear) at Week 8, considering a 2-point decrease from baseline. A sample size of 45 subjects will have a power of 80% to detect a statistically significant difference of 25% between responders from treated group and from the placebo group, based on a chi-square test and an alpha of 0.05. Based on the literature review, it was expected that the placebo could reach up to 7%, so the minimal proportion expected in the treated group was at least 32%. Allowing for 10% drop-out, a total of 100 subjects were planned to be enrolled in the study.

Efficacy was evaluated on the basis of the ITT population and analyses were performed based on the randomized treatment and not on the treatment received. The per-protocol (PP) population included all subjects who were randomized with no significant protocol deviations. The safety population was defined as all subjects who received at least one dose of the medication. Analysis will be done according to the actual treatment they received.

The primary endpoint can be translated as a responder analysis where a subject is classified as Responder if he/she achieves an IGA score of 0 (clear) or 1 (almost clear) at Week 8, considering a 2-point decrease from baseline. The comparison between groups for the primary endpoint was done using a Cochran-Mantel-Haenszel test, with site included as a stratification factor. A supportive analysis was performed using a Fisher's exact test. The primary efficacy analysis was done using observed values and a supportive analysis was conducted using the last observation carried forward (LOCF) approach. The analyses was done using the ITT population and served as the primary analysis, while the analysis of the primary endpoint using the PP population was used as a sensitivity analysis.

The secondary endpoints involving change from baseline were analyzed using an analysis-of-covariance (ANCOVA) including the change from baseline as the dependent, the site and treatment group and site as fixed effects, and the baseline value as covariate. Ls-means and 95% CI were presented along with corresponding p-value from the comparison of treatment. The secondary endpoints involving proportion were analyzed using a Cochran-Mantel-Haenszel test stratified by site and p-value was presented. Analyses for the secondary endpoints were done using observed data and no imputation will be used for missing observation.

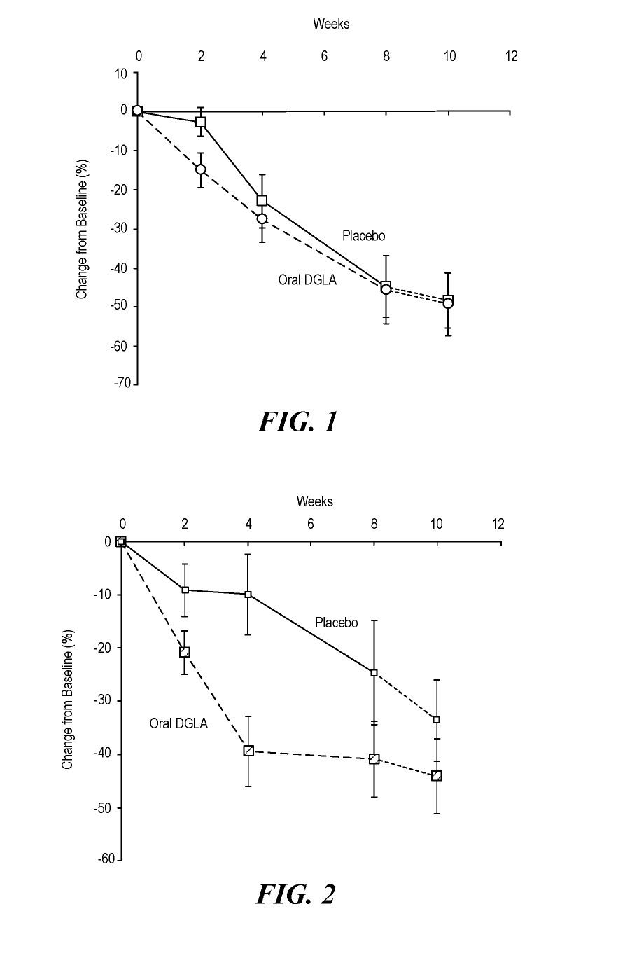

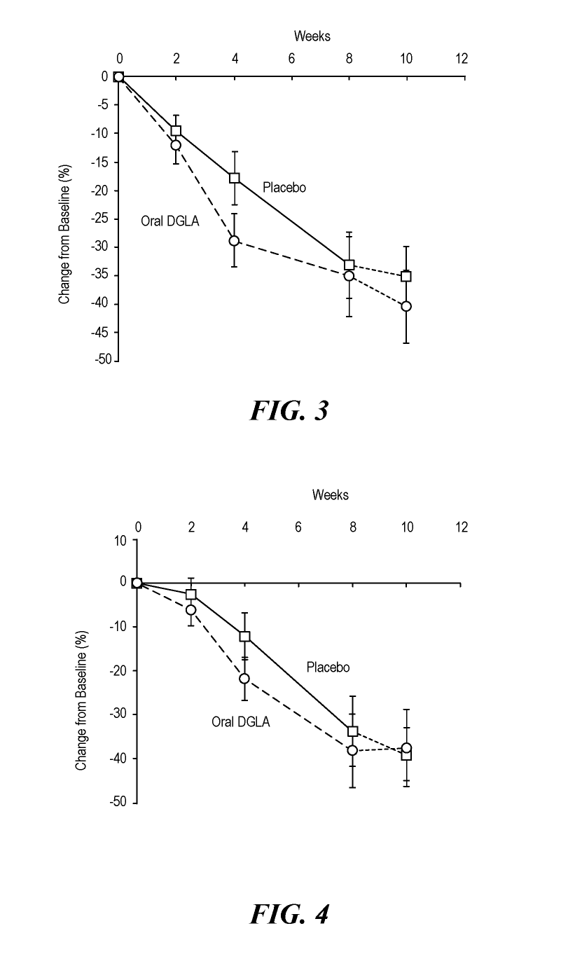

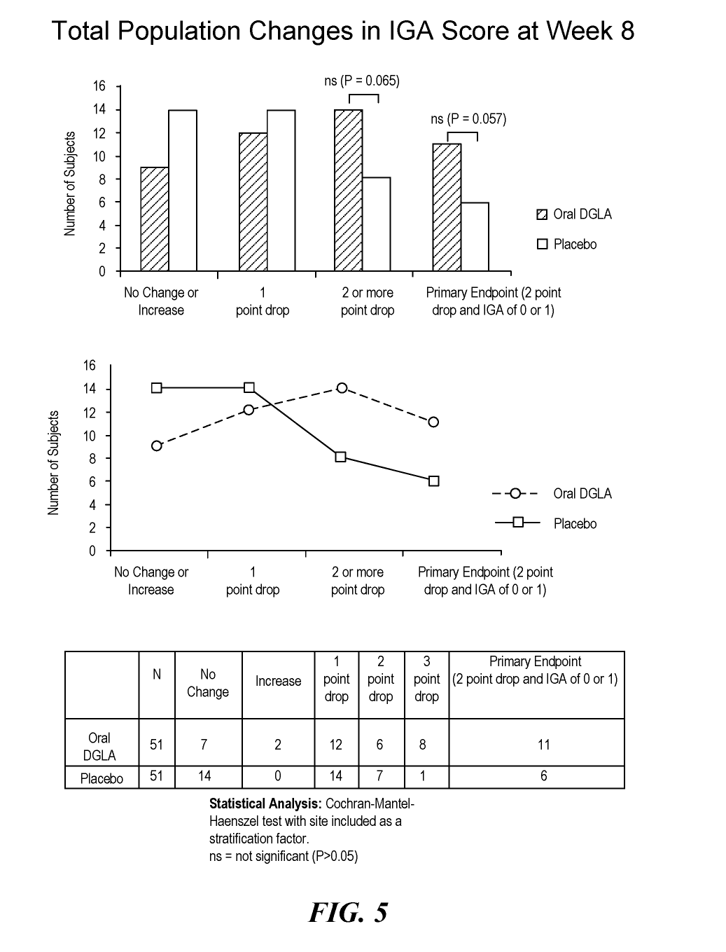

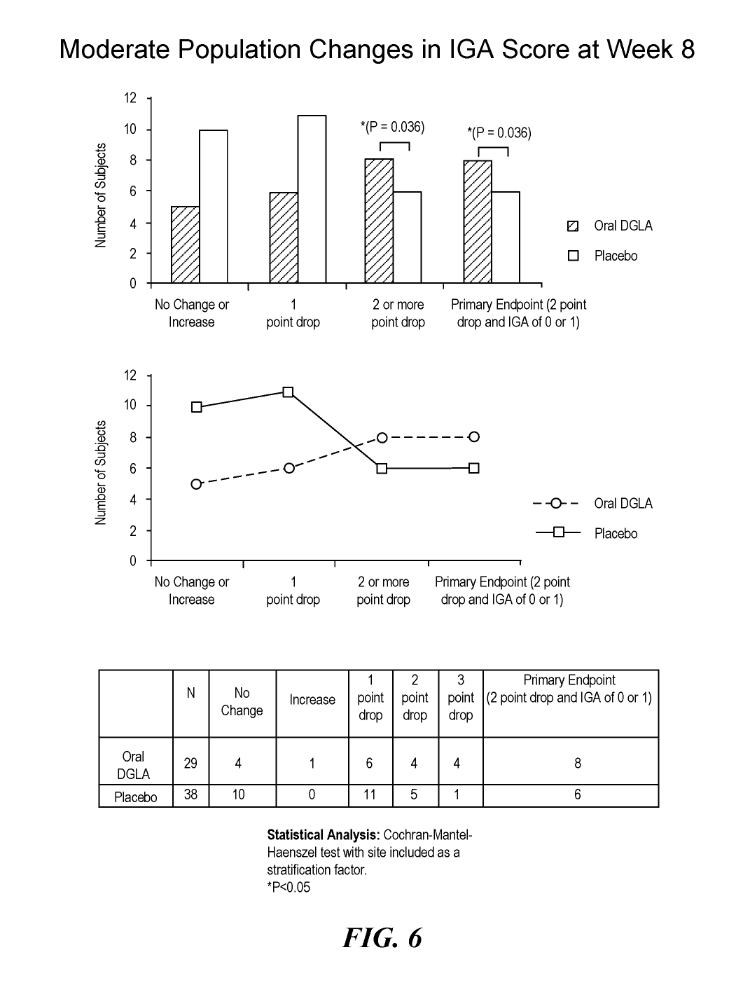

Results of the study are shown in Tables 8-11 and FIGS. 1-6. Based on the FDA standard primary efficacy end point for atopic dermatitis clinical trials (a two point drop in the Investigator Global Assessment (IGA), as well as an end of treatment IGA of 0 (clear) or 1 (almost clear)), the overall results from intent to treat (ITT) analysis showed a clinically significant trend (p=0.057) in favor of DS107 over placebo. In particular, this analysis showed a statistically significant improvement (p=0.036) over placebo in the moderate patient subgroup (n=67).

Among the secondary endpoints that have been assessed, the pruritus score [visual analog scale] showed a statistically significant reduction of itch (p=0.015) within four weeks of dosing in the group treated with DS107.

The safety data indicated that oral DS107 was very well tolerated with a broad safety profile. There were no drug related Serious Adverse Events in either patient group. Most of the adverse events experienced were mild, resolved without any intervention and did not seem to differ between the groups.

Table 8 shows changes in eczema area and severity index from baseline for DGLA and placebo.

TABLE 8

| | | Treatment Group | | |
|---|---|---|---|---|
| | | DS107G | Placebo | Total |
| | | (N = 51) | (N = 51) | (N-102) |
| Parameter | | n | | |
| Week 8/ET | Statistic | 35 | 36 | 71 |
| Actual | Mean | 9.3 | 8.8 | 9.0 |
| | SD | 8.5 | 7.9 | 8.1 |
| | Median | 7.4 | 6.5 | 7.2 |
| | Min, Max | 0, 30 | 0, 29 | 0, 30 |
| | Interquartile | 2.1, 15.0 | 2.8, 12.7 | 2.4, 13.8 |
| Change from | Mean | −9.4 | −8.4 | −8.9 |
| baseline | SD | 11.1 | 9.5 | 10.2 |
| | Median | −8.0 | −6.8 | −7.4 |
| | Min, Max | −38, 18 | −37, 9 | −38, 18 |
| | Interquartile | −15.8, −1.2 | −13.3, −2.5 | −14.2, −1.6 |
| | LSMEANS¹ | −8.0 | −7.7 | |
| | 95% CI | ( −10.8, −5.3) | ( −10.3, −5.0) | |
| | p-value¹ | 0.831 | | |
| Percent | Mean | −45.6 | −44.9 | −45.3 |
| Change from | SD | 51.4 | 46.7 | 48.7 |
| Baseline | Median | −62.7 | −53.6 | −57.7 |
| | Min, Max | −100, 154 | −100, 116 | −100, 154 |
| | Interquartile | −80.9, −7.6 | −79.5, −15.5 | −79.8, −14.5 |

All data expressed as Mean ± SEM (Standard Error of Mean). Treatment stops at week 8 (follow-up at week 10)
¹LSMEANs and p-value from an ANCOVA on the change from baseline. The model includes Treatment group, Site as fixed effects and the baseline value as the covariate
2 LSMEANs and p-value from an ANCOVA on the change from Week 8/ET. The model includes Treatment group, Site as fixed effects and Baseline and (Week 8/ET − Baseline) values as covariates
Note(s):
Baseline is the result measured at Baseline Visit.

Table 9A/B show changes in visual analog scale of pruritus from baseline for DGLA and placebo.

TABLE 9A
(Week 4).

| | | Treatment Group | | |
|---|---|---|---|---|
| Parameter | | DS107G | Placebo | Total |
| Week 8/ET | Statistic | (N = 51) | (N = 51) | (N-102) |
| | | n | | |
| | | 35 | 36 | 71 |
| Actual | Mean | 4.6 | 4.5 | 4.5 |
| | SD | 3.5 | 3.0 | 3.2 |
| | Median | 4.0 | 4.3 | 4.0 |
| | Min, Max | 0, 10 | 0, 10 | 0, 10 |
| | Interquartile | 1.0, 8.0 | 2.3, 7.0 | 1.5, 7.5 |
| | | n | | |
| | | 34 | 36 | 70 |
| Change from | Mean | −2.8 | −2.2 | −2.5 |
| baseline | SD | 3.0 | 3.3 | 3.1 |
| | Median | −2.0 | −2.0 | −2.0 |
| | Min, Max | −8, 3 | −10, 4 | −10, 4 |
| | Interquartile | −5.0, −0.5 | −4.0, −0.3 | −4.7, −0.5 |
| | LSMEANS¹ | −2.1 | −1.5 | |
| | 95% CI | ( −3.1, −1.0) | ( −2.6, −0.5) | |
| | p-value¹ | 0.450 | | |
| Percent Change | Mean | −41.0 | −25.8 | −33.2 |
| from Baseline | SD | 41.6 | 59.0 | 51.5 |
| | Median | −35.3 | −33.3 | −33.3 |
| | Min, Max | −100, 38 | −100, 133 | −100, 133 |
| | Interquartile | −88.2, −7.3 | −67.5, −2.6 | −71.4, −6.0 |

TABLE 9B
(Week 8)

| | | Treatment Group | | |
|---|---|---|---|---|
| Parameter | | DS107G | Placebo | Total |
| Week 8/ET | Statistic | (N = 51) | (N = 51) | (N-102) |
| | | n | | |
| | | 35 | 36 | 71 |
| Actual | Mean | 4.6 | 4.5 | 4.5 |
| | SD | 3.5 | 3.0 | 3.2 |
| | Median | 4.0 | 4.3 | 4.0 |
| | Min, Max | 0, 10 | 0, 10 | 0, 10 |
| | Interquartile | 1.0, 8.0 | 2.3, 7.0 | 1.5, 7.5 |
| | | n | | |
| | | 34 | 36 | 70 |
| Change from | Mean | −2.8 | −2.2 | −2.5 |
| baseline | SD | 3.0 | 3.3 | 3.1 |
| | Median | −2.0 | −2.0 | −2.0 |
| | Min, Max | −8, 3 | −10, 4 | −10, 4 |
| | Interquartile | −5.0, −0.5 | −4.0, −0.3 | −4.7, −0.5 |
| | LSMEANS¹ | −2.1 | −1.5 | |
| | 95% CI | ( −3.1, −1.0) | ( −2.6, −0.5) | |
| | p-value₁ | 0.450 | | |
| Percent Change | Mean | −41.0 | −25.8 | −33.2 |
| from Baseline | SD | 41.6 | 59.0 | 51.5 |
| | Median | −35.3 | −33.3 | −33.3 |
| | Min, Max | −100, 38 | −100, 133 | −100, 133 |
| | Interquartile | −88.2, −7.3 | −67.5, −2.6 | −71.4, −6.0 |

Table 10 shows changes in SCORing of atopic dermatitis from baseline for DGLA and placebo.

TABLE 10

| | | Treatment Group | | |
|---|---|---|---|---|
| Parameter | | DS107G | Placebo | Total |
| Week 8/ET | Statistic | (N = 51) | (N = 51) | (N-102) |
| | | n | | |
| | | 35 | 36 | 71 |
| Actual | Mean | 35.9 | 34.1 | 35.0 |
| | SD | 22.1 | 18.1 | 20.1 |
| | Median | 28.6 | 32.0 | 31.0 |
| | Min, Max | 0, 82 | 1, 86 | 0, 86 |
| | Interquartile | 18.7, 51.0 | 19.9, 47.0 | 19.6, 48.5 |
| | | n | | |
| | | 34 | 36 | 70 |
| Change from | Mean | −21.1 | −18.3 | −19.6 |
| baseline | SD | 21.7 | 18.4 | 20.0 |
| | Median | −21.2 | −18.4 | −18.7 |
| | Min, Max | −60, 33 | −60, 19 | −60, 33 |
| | Interquartile | −39.0, −4.5 | −32.4, 3.7 | −35.4, −4.5 |
| | LSMEANS¹ | −18.7 | −16.6 | |
| | 95% CI | ( −25.4, −12.0) | ( −23.2, −10.1) | |
| | p-value¹ | 0.629 | | |
| Percent | Mean | −36.1 | −33.2 | −34.6 |
| Change from | SD | 40.7 | 34.9 | 37.6 |
| Baseline | Median | −43.4 | −38.1 | −41.8 |
| | Min, Max | −99, 67 | −99, 52 | −99, 67 |
| | Interquartile | −67.1, −7.1 | 56.4, −7.5 | −62.8, −7.1 |

Table 11 shows changes in body surface area affected by atopic dermatitis for DGLA and placebo.

TABLE 11

| | | Treatment Group | | |
|---|---|---|---|---|
| | | DS107G | Placebo | Total |
| | | (N = 51) | (N = 51) | (N-102) |
| Parameter | | n | | |
| Week 8/ET | Statistic | 35 | 36 | 71 |
| Actual | Mean | 17.6 | 16.9 | 17.2 |
| | SD | 18.2 | 17.9 | 17.9 |
| | Median | 12.0 | 10.0 | 10.0 |
| | Min, Max | 0, 80 | 0, 75 | 0, 80 |
| | Interquartile | 5.0, 25.0 | 5.0, 20.8 | 5.0, 25.0 |
| Change from | Mean | −9.6 | −7.0 | −8.3 |
| baseline | SD | 13.8 | 9.6 | 11.9 |
| | Median | −8.0 | −6.5 | −7.0 |
| | Min, Max | −44, 30 | −30, 24 | −44, 30 |
| | Interquartile | −15.0, −2.0 | −13.0, −1.5 | −15.0, −2.0 |
| | LSMEANS¹ | −8.9 | −6.2 | |
| | 95% CI | ( −13.1, −4.7) | ( −10.2, −2.2) | |
| | p-value¹ | 0.318 | | |
| Percent Change | Mean | −38.2 | −33.9 | −36.0 |
| from Baseline | SD | 49.2 | 47.2 | 47.9 |
| | Median | −50.0 | −42.0 | −42.9 |
| | Min, Max | −100, 136 | −100, 147 | −100, 147 |
| | Interquartile | −75.0, −5.9 | −72.4, −4.3 | −75.0, −5.9 |

TABLE 12
Summary of Investigator's Global Assessment—Responder Analysis
at Week 8 (Intent-to-Treat Population)—Total population

| | | Treatment Group | | |
|---|---|---|---|---|
| Parameter | | DS107G | Placebo | Total |
| IGA Score | Statistic | (N = 51) | (N = 51) | (N-102) |
| Baseline | | | | |
| 3—Moderate | n (%) | 29 ( 56.9) | 38 ( 74.5) | 67 ( 65.7) |
| 4—Severe | n (%) | 20 ( 39.2) | 13 ( 25.5) | 33 ( 32.4) |
| 5—Very Severe | n (%) | 2 ( 3.9) | 0 ( 0.0) | 2 ( 2.0) |
| Week 8 | | | | |
| 0—Clear | n (%) | 4 ( 7.8) | 1 ( 2.0) | 5 ( 4.9) |
| 1—Almost Clear | n (%) | 7 ( 13.7) | 5 ( 9.8) | 12 ( 11.8) |
| 2—Mild | n (%) | 9 ( 17.6) | 13 ( 25.5) | 22 ( 21.6) |
| 3—Moderate | n (%) | 10 ( 19.6) | 13 ( 25.5) | 23 ( 22.5) |
| 4—Severe | n (%) | 4 ( 7.8) | 4 ( 7.8) | 8 ( 7.8) |
| 5—Very Severe | n (%) | 1 ( 2.0) | 0 ( 0.0) | 1 ( 1.0) |
| Responders¹ | n (%) | 14 ( 27.5) | 8 ( 15.7) | 22 ( 21.6) |
| Non-Responders | n (%) | 37 ( 72.5) | 43 ( 84.3) | 80 ( 78.4) |
| | p-value² | 0.065 | | |
| | p-value³ | 0.228 | | |

¹Subject is classified as Responder if he/she achieves a decrease of at least 2 points in IGA score at Week 8.
²p-value from a Cochran-Mantel-Haenszel test with site included as a stratification factor.
³p-value from a Fisher Exact test (data from all sites combined).
⁴IGA score at baseline visit.

TABLE 13
Summary of Investigator's Global Assessment—Responder Analysis
at Week 8 (Intent-to-Treat Population)—By group

| | | Treatment Group | | |
|---|---|---|---|---|
| | | DS107G | Placebo | Total |
| Parameter | Statistic | (N = 51) | (N = 51) | (N-102) |
| Week 8—Very | | | | |
| Severe/Severe | | | | |
| 0—Clear | n (%) | 0 (0.0) | 0 (0.0) | 0 (0.0) |
| 1—Almost Clear | n (%) | 3 (13.6) | 0 (0.0) | 3 (8.6) |
| 2—Mild | n (%) | 3 (13.6) | 2 (15.4) | 5 (14.3) |
| 3—Moderate | n (%) | 6 (27.3) | 3 (23.1) | 9 (25.7) |
| 4—Severe | n (%) | 3 (13.6) | 4 (30.8) | 7 (20.0) |
| 5—Very Severe | n (%) | 1 (4.5) | 0 (0.0) | 1 (2.9) |
| Responders¹ | n (%) | 6 (27.3) | 2 (15.4) | 8 (22.9) |
| Non-Responders | n (%) | 16 (72.7) | 11 (84.6) | 27 (77.1) |
| | p-value² | 0.542 | | |
| Week 8—Moderate | | | | |
| 0—Clear | | 4 (13.8) | 1 (2.6) | 5 (7.5) |
| 1—Almost Clear | n (%) | 4 (13.8) | 5 (13.2) | 9 (13.4) |
| 2—Mild | n (%) | 6 (20.7) | 11 (28.9) | 17 (25.4) |
| 3—Moderate | n (%) | 4 (13.8) | 10 (26.3) | 14 (20.9) |
| 4—Severe | n (%) | 1 (3.4) | 0 (0.0) | 1 (1.5) |
| 5—Very Severe | n (%) | 0 (0.0) | 0 (0.0) | 0 (0.0) |
| Responders¹ | n (%) | 8 (27.6) | 6 (15.8) | 14 (20.9) |
| Non-Responders | n (%) | 21 (72.4) | 32 (84.2) | 53 (79.1) |
| | p-value² | | | |

A surprising discovery was observed when the data was further interrogated. In particular, it was found that subjects having low baseline eosinophil counts exhibited a higher response rate to DS107 treatment.

Eosinophils are a type of white blood cell that play an important role in the body's response to allergic reactions, asthma, and infection with parasites. Eosinophils usually account for less than 7% of the circulating white blood cells (100 to 500 eosinophils per microliter of blood). These cells have a role in the protective immunity against certain parasites but also contribute to the inflammation that occurs in allergic disorders.

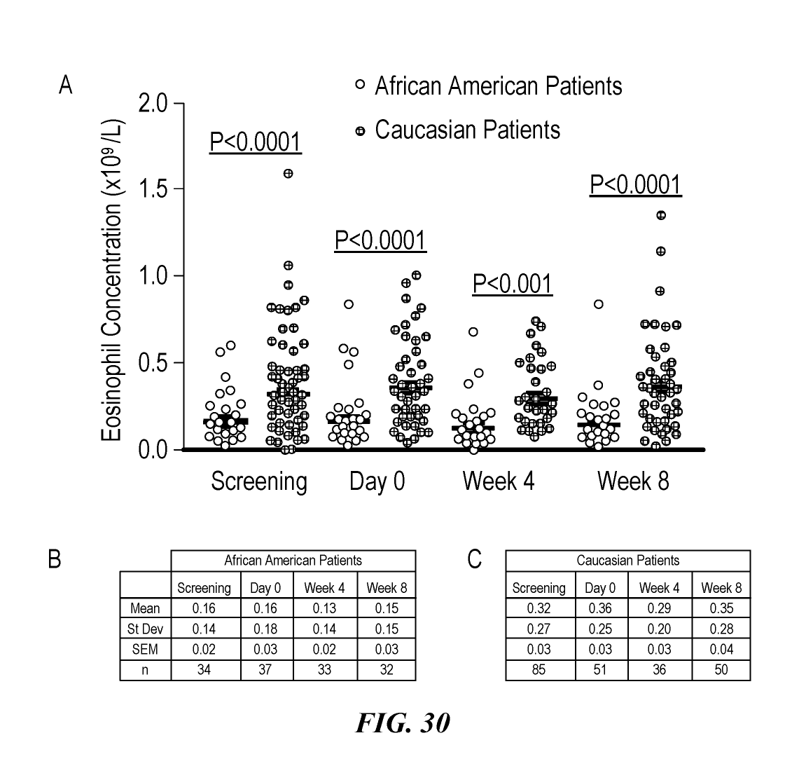

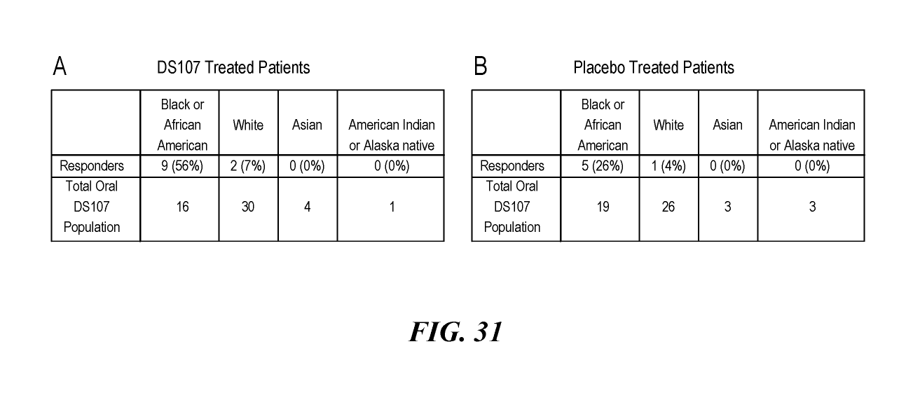

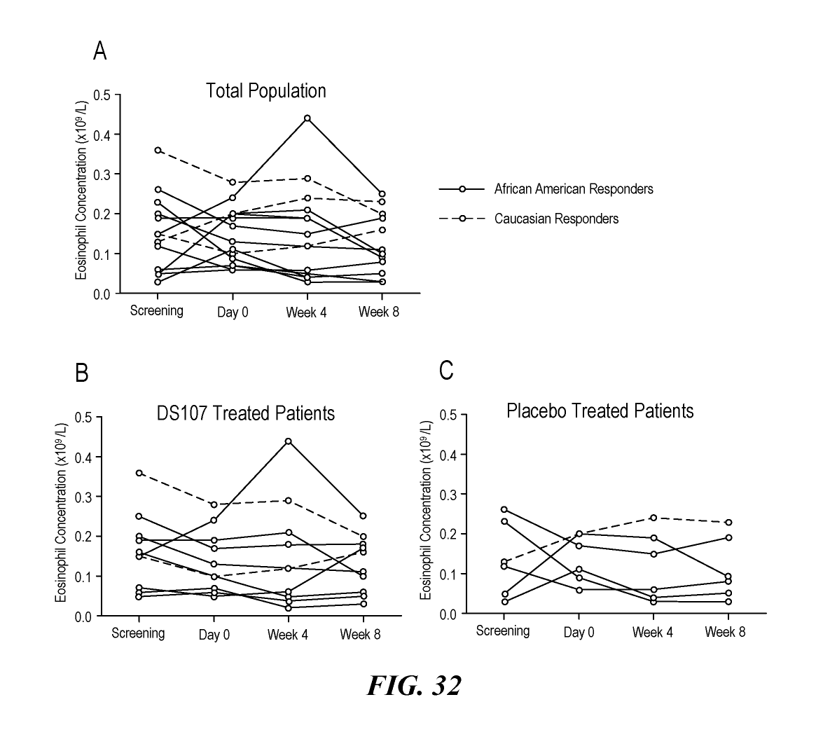

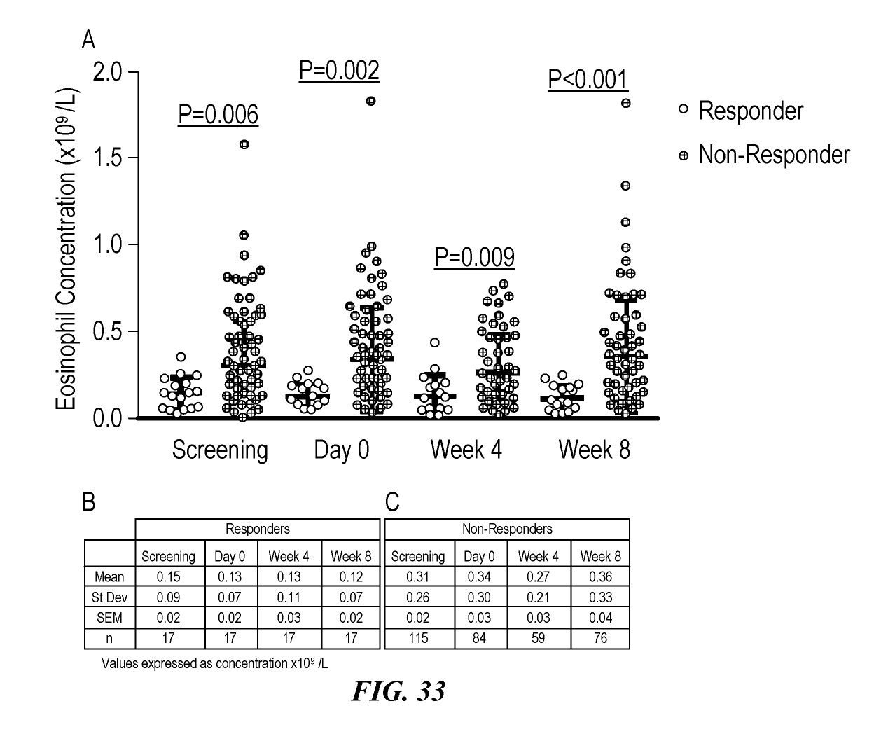

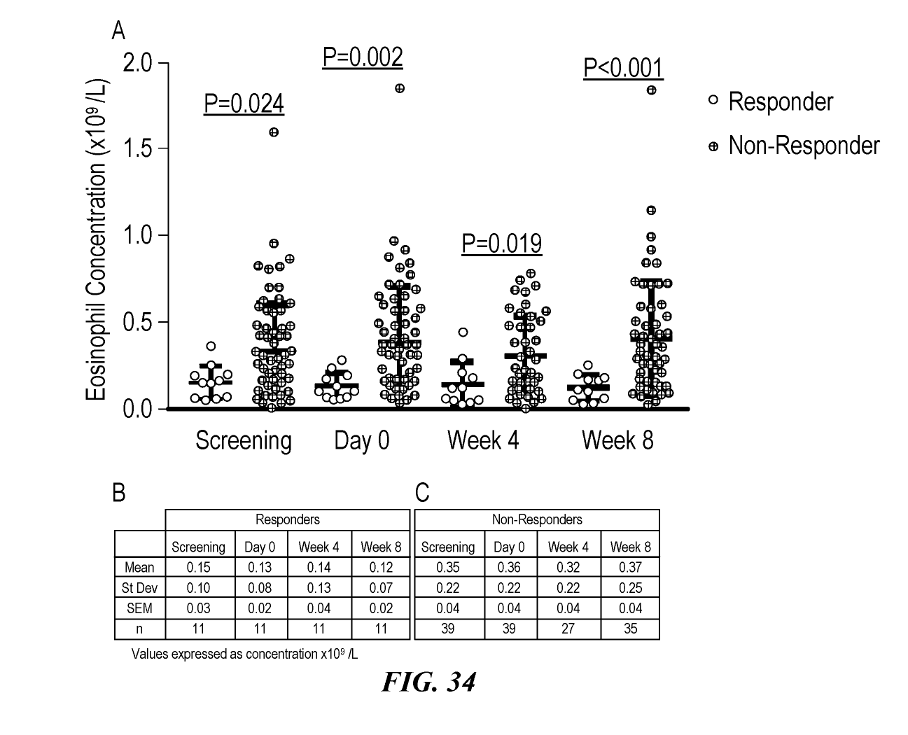

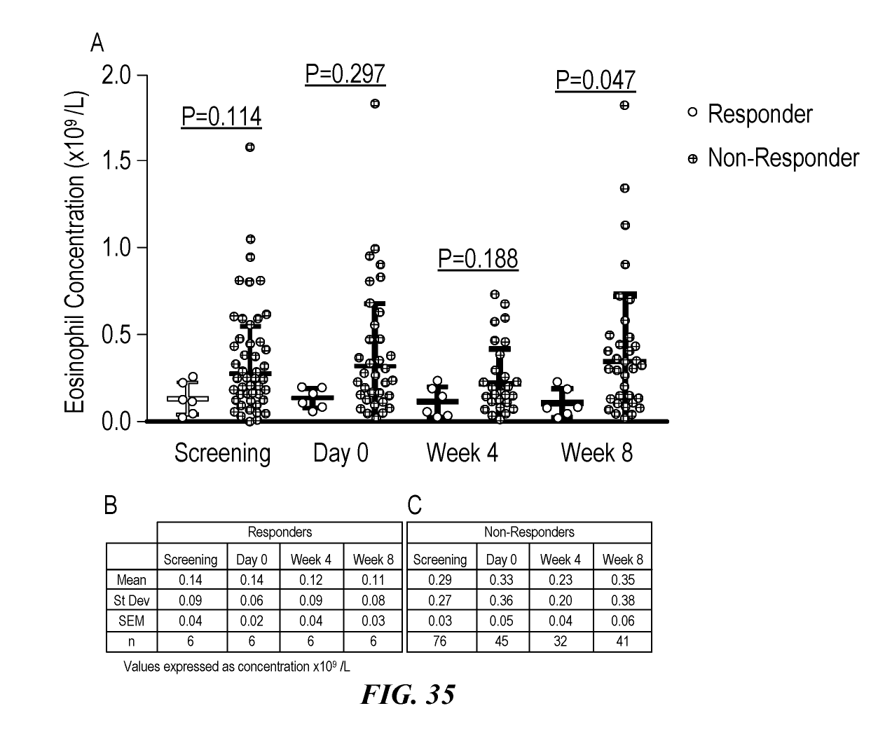

Results of the analyses are shown in FIGS. 30A-35C. The data shows statistically significant reductions in eosinophil concentrations in African American patients, as compared to Caucasian patients, treated with DS107 at each time point measured. FIG. 30A-B. In particular, 56% of DS107-treated African America patients were classified as Responders, versus only 7% of Caucasian patients. FIG. 31A-B. As discussed in detail above, a subject is classified as a Responder if he/she achieves an IGA score of 0 (clear) or 1 (almost clear) at Week 8, considering a 2-point decrease from baseline. FIG. 32 depicts the same data for eosinophil concentrations in all Responders in the total population (FIG. 32A) and for DS107 treated patients (FIG. 32B) and placebo treated patients (FIG. 32C). In addition, data showed a statistically significant decrease in eosinophil concentration at all time points measured in the total population (Responders and Non-responders). FIG. 33A-C. Finally, the data also revealed a statistically significant decrease in eosinophil concentration at all time points in DS107-treated Responders, as compared to Non-responders versus placebo controls. FIGS. 34A-C and FIGS. 35A-C.

Together these data suggest that patients pre-determined to have low eosinophil levels represent likely candidates for treatment with DGLA, in particular an oral DGLA such as DS107. Such findings are useful in predicting efficacy of a DGLA therapy for patients.

### Example 3

An in vivo investigation of anti-hypertension efficacy following co-administration of DGLA and aspirin in spontaneous hypertensive rats was conducted. This experiment investigated the efficacy of DGLA when co-administered chronically with aspirin to reduce the hypertensive response induced acutely by phenylephrine in spontaneous hypertensive rats. Spontaneously hypertensive adult male rats (250-300 g), bred by Charles River Laboratories, were used for this assay. The animals were identified upon arrival as per CCPA guidelines. The animals were pair-housed by group (low dose+aspirin, high dose+aspirin or aspirin alone) for the duration of the oral gavage prior to surgery. All animal care and vivarium maintenance was recorded, with documents kept at the test facility.

The rat is a well-characterized and highly sensitive model for assessment of vascular tension effects and evaluation of efficacy following chronic exposure to a test article. This study design is based on current International Conference on Harmonization (ICH) Harmonized Tripartite Guidelines [ICH S7A] and generally accepted procedures for the testing of pharmaceutical compounds. This nonclinical laboratory study was designed as a non-GLP compliant study and did not require QA involvement.

The concentrations of DGLA to be tested (50 and 500 mg/kg) as well as the concentration of aspirin (10 mg/kg) were selected by the Sponsor based on information available at the time of design of this study. They were selected to cover a range of concentrations useful for the identification of the mechanism of action of the test article.

The concentrations of positive control (carvedilol 0.1 mg/kg) and the hypertensor (phenylephrine 3, 10, and 20 μg/kg) were selected based on literature references. All solution preparations were documented on a Solution Contents Form, defining labelling information and all relevant information on reagents: batch number, storage conditions, contents, and preparation/expiry dates.

The test article was formulated using olive oil as the vehicle. This stock solution was then administered by oral gavage once per day for seven consecutive days. A low and a high dose (50 mg/kg and 500 mg/kg) were tested.

A 5 mg/ml stock solution of aspirin was prepared by dissolving the appropriate amount of aspirin in 100% DMSO which was then diluted in water. The concentration of DMSO was less than 1%. The appropriate volume was administered by oral gavage to each animal once per day for seven consecutive days. The stock solution was stored at room temperature and was considered stable for the duration of the gavage.

A 0.4 mg/m I stock solution of carvedilol was prepared in PBS (pH=4.00±0.05). The appropriate volume was administered to each animal by an intravenous injection at the end of the experiment on the surgery day. The stock solution was stored at 4° C. and was considered stable for the duration of the experiment.

A stock solution of phenylephrine of 1 mg/ml were prepared in PBS (pH=7.4±0.05). The stock solution was stored at 4° C. The expiration date was set at 14 days after preparation.

Any remaining test article will be destroyed after completion of the study.

Male spontaneous hypertensive rats weighing approximately 300 g arrived at the facility at least two days before beginning the oral gavage. Animals were assigned to one of three groups (low dose+aspirin, high dose+aspirin, and aspirin), and were pair-housed during the acclimation period. Each group (n=4) received the appropriate compound by oral gavage using a 16 G feeding needle. The stock solution was administered once per day for seven consecutive days. On the seventh day of administration, the animals underwent surgery and hypertensive monitoring.

Rats were anesthetized with a 2.5-3.0% isoflurane-O2 mixture in an induction box. When the animals were removed from the induction box, they were connected onto a nose cone to maintain anesthesia during the tracheotomy. The animals were tracheotomised with an endotracheal tube (7 cm length from connector to the tip made with a PE205 tube from BD and a 16 G needle) to facilitate spontaneous breathing, stabilize hemodynamics, and to keep the rat under anesthesia with an isoflurane-O2 mixture. Two catheters were inserted: one in the right femoral artery for systemic arterial pressure (SAP) measurement, and one in the right femoral vein for delivery of the hypertensor and positive control. ECG signals were acquired with ECG contacts placed in a Lead-1 configuration on the anaesthetized animal, and a pulse oxymeter was attached to the animal's hind paw to enable continuous monitoring of the general condition of the rat during the surgical procedure. Following baseline measurements, three doses of phenylephrine (3, 10 and 20 μg/kg) were delivered by an IV bolus injection, leaving 5 minutes between doses. One dose of the positive control, carvedilol (0.1 mg/kg), was administered, following the highest concentration of phenylephrine. After the positive control, 10 μg/kg of phenylephrine was administered again. Blood pressure, systemic arterial pressure (SAP), and heart rate were monitored continuously for a total of half an hour. Hypertension was detected as an increase in diastolic, systolic or mean blood pressure. At the end of the experiment, the animals were euthanized by exsanguination.

The analysis software used was Clampfit 10.2.0.14 by Axon Instruments, installed on networked personal computers running Microsoft Windows. Clampfit 10.2.0.14 has been fully validated in the connected context in which it is used. The graphics software for illustrations is Microsoft Office Excel 2007 installed on networked personal computers running Microsoft Windows. The continuously recorded systemic arterial pressure (SAP) was used to calculate the mean systemic arterial pressure (mSAP) for each condition. The heart rate was monitored continuously from the time anesthesia was induced. One-way ANOVAs, comparing pre- and post-exposure parameters across experimental groups, were analyzed. Statistical significance was confirmed at p≤0.05.

TABLE 14
Arterial pressure and mean systemic arterial pressure (mmHg)
following seven days of gavage, before intravenous doses of
phenylephrine.

| | | Mean systemic |
|---|---|---|
| Treatment | Systol/Diastol | arterial pressure |
| Olive Oil (vehicle) | 160/100 | 120 |
| DGLA 50 mg/kg | 120/75 | 90 |
| DGLA 500 mg/kg | 135/80 | 98 |
| Aspirin 10 mg/kg | 190/100 | 130 |
| DGLA 50 mg/kg + | 180/120 | 140 |
| Aspirin 10 mg/kg | | |
| DGLA 50 mg/kg + | 180/120 | 140 |
| Aspirin 10 mg/kg | | |

*Compared to baseline
** Compared to PHE 20 μg/kg
† Compared to second baseline
§ Compared to Carvedilol

Table 15 shows change in mean systemic arterial pressure with intravenous phenylephrine doses following seven consecutive days of gavage with aspirin at 10 mg/kg/day.

TABLE 15
Treatment Aspirin 10 mg/kg
Mean Arterial Pressure

| | | | | | | Average | | | | |
|---|---|---|---|---|---|---|---|---|---|---|
| Conditions | Rat #1 | Rat #2 | Rat #3 | Rat #4 | Rat #5 | (mmHg) | STED | SEM | Test vs baseline | n |
| Baseline | 86.75 | 107.64 | 160.50 | 141.36 | 187.84 | 136.8 | 40.44 | 18.09 | N/A | 5 |
| PHE 3 μg/kg | 97.24 | 122.18 | 168.06 | 150.54 | 192.06 | 146.0* | 37.33 | 16.69 | 0.006 | 5 |
| PHE 10 μg/kg | 137.63 | 122.18 | 203.75 | 169.61 | 201.69 | 167.0* | 36.85 | 16.48 | 0.016 | 5 |
| PHE 20 μg/kg | 149.35 | 120.84 | 202.61 | 194.76 | 203.90 | 174.3* | 37.34 | 16.70 | 0.019 | 5 |
| Baseline | 98.38 | 64.32 | 164.88 | 159.32 | 177.61 | 132.9** | 49.03 | 21.93 | 0.002 | 5 |
| Carvedilol 0.1 mg/kg | 57.12 | 44.38 | 145.56 | 134.82 | 115.06 | 99.4 † | 45.95 | 20.55 | 0.015 | 5 |
| PHE 10 μg/kg | 93.22 | 52.61 | 185.63 | 171.45 | 187.96 | 138.2 § | 61.60 | 27.55 | 0.020 | 5 |

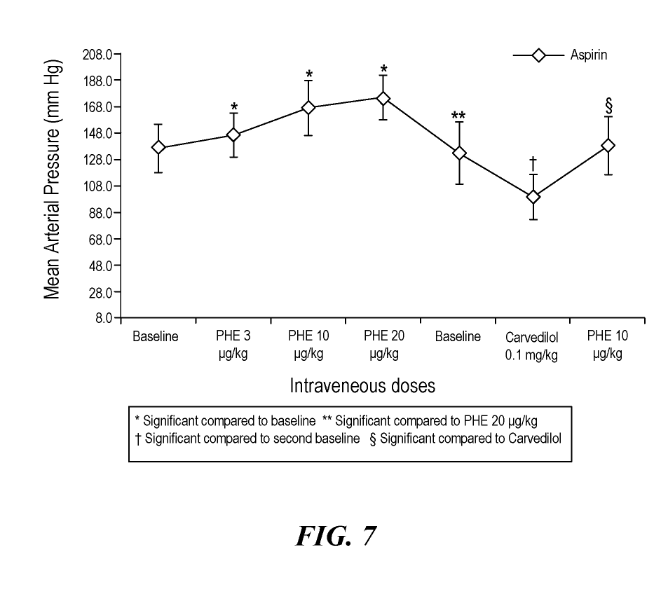

FIG. 7 illustrates the change in mean systemic arterial pressure, in mm of Hg, following phenylephrine administration. Five rats received intravenous doses of phenylephrine after seven consecutive days of gavage with aspirin at 10 mg/kg/day. The mean systemic arterial pressure at each of the three doses of phenylephrine was compared statistically to the first baseline, while the carvedilol condition was statistically compared to the baseline immediately prior to administration, and the final dose of phenylephrine (10 μg/kg) was compared to the positive control condition directly before.

These results demonstrate an increased mean systemic arterial pressure with increasing doses of phenylephrine. All data obtained were statistically different from baseline, confirming the hypertensor effect of phenylephrine in the spontaneously hypertensive rat model fed with aspirin. The effect of phenylephrine was fully reversible; the mean arterial pressure during the second baseline (following 20 μg/kg) was actually quite similar to the original baseline.

Carvedilol caused a significant decrease when added immediately after phenylephrine, confirming its well-known ability to reduce high blood pressure (carvedilol is a non-specific beta blocker/alpha-1 blocker and blocks the binding of phenylephrine to alpha-1 adrenergic receptors). The last addition of phenylephrine, following the carvedilol, caused a limited increase in the mean systemic arterial pressure.

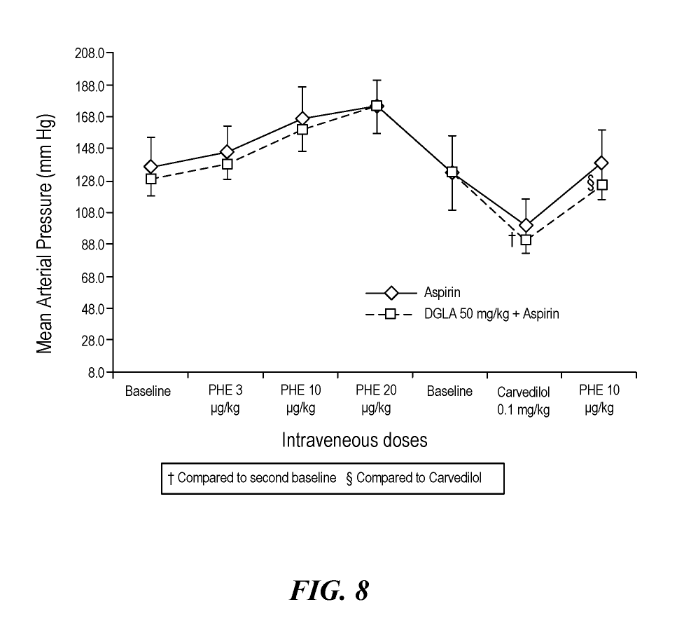

FIG. 8 shows the change in mean arterial pressure (mmHg) with intravenous doses of phenylephrine following seven consecutive days of gavage with DGLA at 50 mg/kg+aspirin at 10 mg/kg.

Table 16 shows the change from baseline in mean arterial pressure with intravenous phenylephrine doses following seven consecutive days of gavage with DGLA at 50 mg/kg+aspirin at 10 mg/kg.

TABLE 16
Treatment 50 mg/kg + Aspirin 10 mg/kg
Mean Arterial Pressure

| | | | | | | | | | Ttest | |
|---|---|---|---|---|---|---|---|---|---|---|
| | | | | | | Average | | | vs | |
| Conditions | Rat #1 | Rat #2 | Rat #3 | Rat #4 | Rat #5 | SAP | STED | SEM | Aspirin | n |
| Baseline | 136.52 | 148.19 | 176.99 | 108.14 | 77.13 | 129.4 | 38.25 | 17.11 | 0.773 | 5 |
| PHE 3 μg/kg | 154.55 | 154.66 | 185.15 | 117.40 | 81.18 | 138.6 | 40.08 | 17.93 | 0.770 | 5 |
| PHE 10 μg/kg | 192.63 | 188.72 | 193.26 | 130.48 | 94.43 | 159.9 | 45.19 | 20.21 | 0.793 | 5 |
| PHE 20 μg/kg | 196.59 | 196.00 | 196.67 | 148.23 | 141.78 | 175.9 | 28.25 | 12.64 | 0.942 | 5 |
| Baseline | 169.28 | 160.97 | 160.79 | 94.77 | 78.64 | 132.9 | 42.68 | 19.09 | 1.000 | 5 |
| Carvedilol 0.1 mg/kg | 115.29 | 97.01 | 126.33 | 59.10 | 58.93 | 91.3 † | 31.30 | 14.00 | 0.006 | 5 |
| PHE 10 μg/kg | 178.71 | 160.97 | n/d | 86.29 | 74.07 | 125.0 § | 52.51 | 26.25 | 0.043 | 4 |

The results presented in FIG. 8 demonstrate changes in mean arterial pressure, in mm of Hg, after intravenous doses of phenylephrine in five spontaneously hypertensive rats following seven days of oral feeding with DGLA at 50 mg/kg+aspirin at 10 mg/kg. The results for the test article were compared to the data obtained with the aspirin-only group (10 mg/kg). Carvedilol was compared to the second baseline, and the last dose of phenylephrine was statistically compared to the carvedilol.

The animals exhibited a dose-dependent increase in blood pressure as phenylephrine concentration was increased. The rats that had been administered DGLA 50 mg/kg and aspirin exhibited an increase in arterial pressure for a given dose of phenylephrine which was essentially the same as that measured in the rats having received aspirin over 10 days. With the rats that were fed with DGLA at 50 mg/kg in addition of 10 mg/kg of aspirin, there was no significant difference in the mean arterial pressure when compared to the aspirin group at any of the concentrations of phenylephrine. It would thus appear that daily doses of aspirin prevent the benefits obtained from daily doses of 50 mg/kg DGLA.

Carvedilol caused a significant decrease in blood pressure in DGLA+aspirin-fed animals. Due to a problem with the anesthesia, rat #3 was not given the last dose of phenylephrine (n=4 for this condition).

Table 17 shows change in mean arterial pressure with intravenous phenylephrine doses following seven consecutive days of gavage with DGLA at 500 mg/kg co-administered with aspirin at 10 mg/kg.

TABLE 17

Treatment
500 mg/kg + Aspirin 10 mg/kg

Mean Arterial Pressure

| | | | | | | | | Test | |
|---|---|---|---|---|---|---|---|---|---|
| | | | | | Average | | | vs | |
| Conditions | Rat #1 | Rat #2 | Rat #3 | Rat #4 | SAP | STED | SEM | Aspirin | n |
| Baseline | 145.62 | 100.15 | 128.35 | 129.60 | 125.9 | 18.90 | 9.45 | 0.638 | 4 |
| PHE 3 μg/kg | 161.09 | 103.72 | 136.18 | 133.59 | 133.6 | 23.49 | 11.74 | 0.584 | 4 |
| PHE 10 μg/kg | 170.69 | 111.08 | 145.53 | 149.12 | 144.1 | 24.66 | 12.33 | 0.325 | 4 |
| PHE 20 μg/kg | 161.17 | 116.35 | 141.85 | 140.72 | 140.0 | 18.36 | 9.18 | 0.140 | 4 |
| Baseline | 120.16 | 83.43 | 88.30 | 85.82 | 94.4 | 17.27 | 8.64 | 0.182 | 4 |
| Carvedilol 0.1 mg/kg | 62.13 | 57.56 | 57.32 | 52.11 | 57.3 † | 4.10 | 2.05 | 0.014 | 4 |
| PHE 10 μg/kg | 95.50 | 65.19 | 80.39 | 58.97 | 75.0 | 16.36 | 8.18 | 0.070 | 4 |

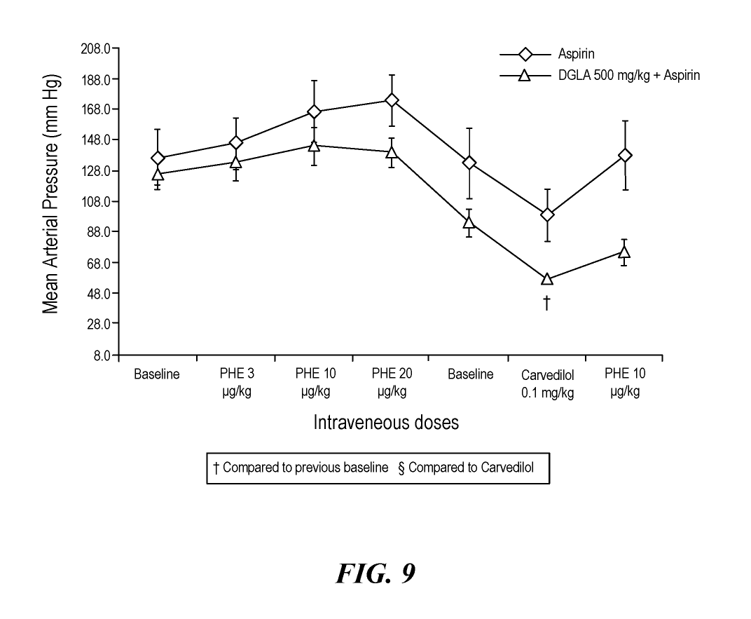

FIG. 9 illustrates the changes in mean arterial pressure following intravenous doses of phenylephrine after seven consecutive days of gavage with DGLA 500 mg/kg co-administered with aspirin at 10 mg/kg in four spontaneously hypertensive rats. As with the lower dose of DGLA+aspirin, the phenylephrine conditions were compared to those of the aspirin-only group. Carvedilol was statistically compared to the baseline just before administration, while the last dose of phenylephrine (10 μg/kg) was compared to the positive control.

Despite lower mean arterial pressures observed, there was no significant statistical difference between the mean systemic arterial pressure of the rats that were fed with both DGLA and aspirin and those that were fed with aspirin only. It would thus appear that aspirin again prevented the benefit of daily administration of 500 mg/kg DGLA. The positive control succeeded in statistically decreasing the arterial blood pressure of the animals, as observed in the other groups. The increase caused by the last dose of phenylephrine was not considered significant.

Table 18 shows mean arterial pressure at baseline following seven consecutive days with six different gavage groups.

TABLE 18

| | Mean Arterial pressure at baseline | | | | | | | | | |
|---|---|---|---|---|---|---|---|---|---|---|
| | | | | | | Average | | | | |
| Conditions | Rat #1 | Rat #2 | Rat #3 | Rat #4 | Rat #5 | SAP | STED | SEM | Ttest | n |
| Vehicle | 70.64 | 77.31 | 120.52 | 93.60 | n/a | 90.5 | 22.21 | 11.10 | n/a | 4 |
| DGLA 50 mg/kg | 51.77 | 87.81 | 62.11 | 86.01 | 84.93 | 74.5 | 16.50 | 7.38 | n/a | 5 |
| DGLA 50 mg/kg + | 136.52 | 148.19 | 176.99 | 108.14 | 77.13 | 129.4 * | 38.25 | 17.11 | 0.019 | 5 |
| Aspirin | | | | | | | | | | |
| DGLA 500 mg/kg | 64.81 | 91.55 | 94.41 | 66.56 | 93.64 | 82.2 | 15.12 | 6.76 | n/a | 5 |
| DGLA 500 mg/kg + | 145.62 | 100.15 | 128.35 | 129.60 | n/a | 125.9 * | 18.90 | 9.45 | 0.006 | 4 |
| Aspirin | | | | | | | | | | |
| Aspirin | 86.75 | 107.64 | 160.50 | 141.36 | 187.84 | 136.8 | 40.44 | 18.09 | n/a | 5 |

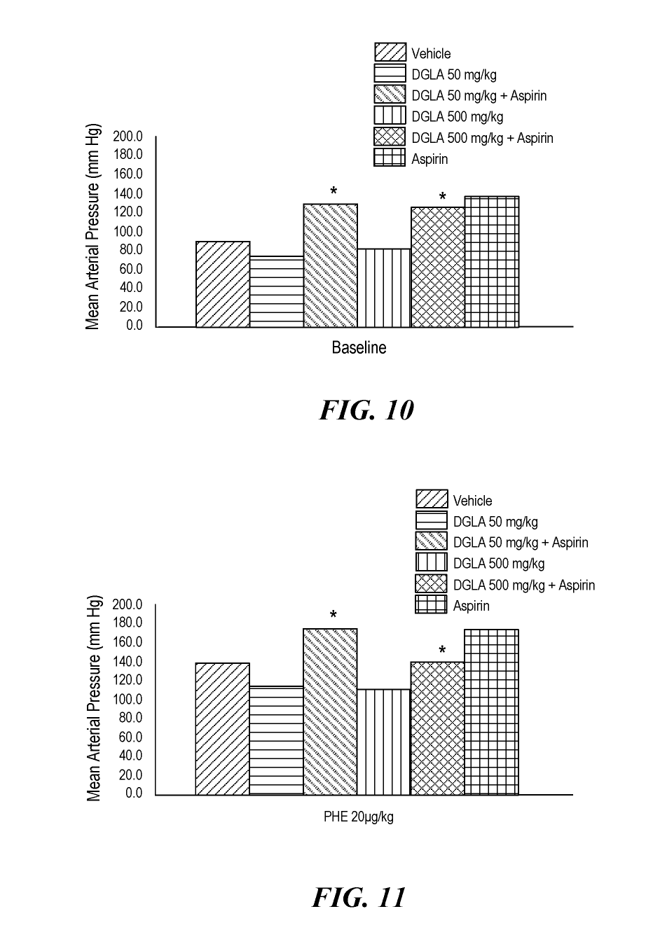

FIG. 10 presents the mean arterial pressures at baseline of the six groups which were fed during the two parts of this study. The tracheotomy stabilized hemodynamics, but also caused changes in the arterial pressure of the rats. The arterial pressure of the rats who did not receive aspirin (Vehicle, DGLA 50 mg/kg and DGLA 500 mg/kg) were obtained during the first phase of this study (study#20131022-1) and those of the other groups were recorded during this present study (different batches of animals from Charles River, but same strain and size of rat). Statistical t tests were done between the groups which were administered DGLA at 50 or 500 mg/kg and their equivalent added with aspirin at 10 mg/kg. There was a statistically significant increase of the mean arterial pressure at baseline between the 50 mg/kg of DGLA alone and the 50 mg/kg of DGLA co-administered with aspirin at 10 mg/kg, as well as between the 500 mg/kg group of DGLA compared to DGLA 500 mg/kg with aspirin 10 mg/kg (significant increase marked with *). It would thus appear that DGLA successfully decreases the arterial pressure in spontaneously hypertensive rats, an effect which is eliminated by aspirin.

Table 19 shows mean arterial pressure with an intravenous dose of phenylephrine at 20 μk/kg following seven consecutive days with six different gavage groups.

TABLE 19

| | Mean Arterial pressure with an intravenous dose of Phenylephrine 20 μg/kg | | | | | | | | | |
|---|---|---|---|---|---|---|---|---|---|---|
| | | | | | | Average | | | | |
| Conditions | Rat #1 | Rat #2 | Rat #3 | Rat #4 | Rat #5 | SAP | STED | SEM | Ttest | n |
| Vehicle | 131.99 | 130.56 | 145.47 | 137.32 | n/a | 136.3 | 6.75 | 3.37 | n/a | 4 |
| DGLA 50 mg/kg | 91.82 | 137.84 | 105.44 | 132.68 | 110.72 | 115.7 | 19.23 | 8.60 | n/a | 5 |
| DGLA 50 mg/kg + | 196.59 | 196.00 | 196.67 | 148.23 | 141.78 | 175.9 * | 28.25 | 12.64 | 0.004 | 5 |
| Aspirin | | | | | | | | | | |
| DGLA 500 mg/kg | 105.21 | 119.33 | 118.12 | 97.17 | 120.82 | 112.1 | 10.43 | 4.66 | n/a | 5 |
| DGLA 500 mg/kg + | 161.17 | 116.35 | 141.85 | 140.72 | n/a | 140.0 * | 18.36 | 9.18 | 0.023 | 4 |
| Aspirin | | | | | | | | | | |
| Aspirin | 149.35 | 120.84 | 202.61 | 194.76 | 203.90 | 174.3 | 37.34 | 16.70 | n/a | 5 |

FIG. 11 presents the mean arterial pressures of all the groups analyzed over the two studies investigating DGLA. These arterial pressures were obtained after an intravenous dose of phenylephrine at 20 μg/kg. As with the results acquired in baseline, each dose of DGLA was statistically compared to its equivalent condition in the aspirin-only group. A significant augmentation of the mean arterial pressure was noticed between both corresponding groups (marked with *).

This study was designed to investigate the efficacy of DGLA, alone and when co-administered chronically with aspirin, to reduce the arterial pressure measured in hypertensive rats, as well as the reduction in the hypertensive response induced acutely by phenylephrine in the same strain of spontaneously hypertensive rats.

The Spontaneously Hypertensive Rat (SHR) is a commonly used model in hypertension research because it produces, within each colony, uniform changes in response to direct and indirect effectors to the cardiovascular system. The rats have been selected and inbred over generations, defined as hypertensive by a systolic blood pressure of over 150 mmHg persisting for more than one month.

The study revealed that the chronic co-administration of DGLA and aspirin prevented DGLA from lowering the mean systemic arterial blood pressure (by up to 25%) as observed previously in the spontaneous hypertensive rats treated with the DGLA.

The doses of DGLA tested (50 and 500 mg/kg) combined with aspirin at 10 mg/kg did not significantly reduce the dose-dependent elevation of the mean arterial blood pressure following phenylephrine injection, when compared to animals which were fed with aspirin alone. The two doses of DGLA caused similar effects, suggesting that the benefits of greater doses of DGLA may be minimal if aspirin is co-administered. When both doses of DGLA were compared to their equivalent aspirin-only conditions, aspirin caused a clear increase in the mean arterial pressure in baseline condition as well as when the rats were challenged with an intravenous dose of 20 μg/kg of phenylephrine.

In this study, Carvedilol was used as the positive control. It is a beta-blocker used to treat heart failure and high blood pressure. It acts by relaxing blood vessels and slowing heart rate, thus improving ventricular refilling, blood flow, and decreasing blood pressure. The effect of carvedilol, when injected intravenously in spontaneously hypertensive rats, confirmed its ability to decrease blood pressure after each of three doses of phenylephrine, and demonstrated the sensitivity of the test system.

### Example 4

A single-center, double-blind, randomized, placebo-controlled, two part Phase one study was performed in healthy male and female subjects aged 18 to 45 years inclusive. The primary objective of this study was to assess the safety and plasma pharmacokinetics (PK) of orally administered single doses and orally administered multiple doses of DS107G capsules (once daily for 28 days) in healthy subjects.

The secondary objectives of this study were to assess the following:

The effect of food on the PK of an orally administered single dose of DS107G capsule in healthy subjects; and

The PK of DGLA in human skin following multiple doses of DS107G capsule given once daily for 28 days to healthy subjects.

A total of 48 subjects were enrolled (including 32 for the single dose study and 16 for the multiple dose study). The single and multiple dose studies consist of 8 subjects per cohort (ideally 4 males and 4 females, but no fewer than 3 per sex).

Subjects were randomly assigned in a 3:1 ratio of DG107G to placebo by block randomization. Single dose part one of the study comprised three cohorts of eight subjects each, with the addition of Cohort four based on evaluation of the safety data of Cohort three by the Safety Monitoring Committee (SMC). A single oral dose of DS107G or matching placebo was administered to fasted subjects in Cohorts one to three (500, 1000, and 2000 mg, respectively) in parallel; subjects in Cohort four were administered a dose of 4000 mg following completion of Cohort three. Subjects in Cohort two received 1000 mg or matching placebo in the fed state at 14 days after the first dose.

Multiple dose part two of the study comprised two cohorts (Cohorts five and six) of eight subjects each, who were administered multiple doses of study drug in the fasted state for 28 days. Subjects in Cohorts five and six received DS107G at 2000 or 4000 mg, respectively, or matching placebo once daily for 28 days. Cohort six was started after Cohort five's safety data were evaluated by the SMC. No interim analysis was planned.

In Part one of the study, plasma PK analysis of Dihomo-Gamma-Linolenic Acid (DGLA) and 15-Hydroxyeicosatrienoic acid (15-HETrE), in fasted subjects alone, was performed for Cohorts one to four up to 312 hours post dose on study Days 1 to 5, 8, 14, and the follow-up visit. In Part two, plasma PK analysis of DGLA was performed for Cohorts five and six up to 168 hours post dose on study Days 1, 2, 3, 5, and 8. Safety assessments were monitored throughout the study.

A randomized, placebo-controlled, double blind design was used to minimize bias during the safety and tolerability assessments.

Part one of the study followed a single ascending dose design with a dose escalation to 4000 mg. With the exception of the 4000-mg dose, all of the doses in Part one of the study were previously tested in humans. Treatment-related adverse events (TEAEs) were not observed in the previously tested multiple-dose regimens such as 150 mg/day for 28 days, 450 mg/day for 28 days, 1000 mg/day for 14 days, and 2000 mg/day for 10 days. The dose selection was based on the doses of oral DGLA tested in previous clinical studies and the characterization of the PK and safety of previously tested and higher doses were observed as the main objective.

The effect of food on the oral bioavailability of DGLA was also assessed.

Healthy female subjects were included in this study to assess a potential gender component on the biomarker di-hydrotestosterone, as DGLA is involved in steroid metabolism.

The total duration of participation for each subject in Part one of the study was approximately 14 days, excluding the screening period. In Part 2 of the study, the duration was approximately 42 days.

Screening procedures were conducted for both parts one and two before the start of the study on Day −1 (Baseline) by evaluating the safety assessments. Safety assessments included adverse events (AEs), clinical laboratory testing (hematology, biochemistry, virology [hepatitis B surface antigen, human immunodeficiency virus (HIV) antibodies, hepatitis C antibodies], and urine analysis), drug of abuse (DOA) test results, pregnancy tests for female subjects (urine ß human chorionic gonadotropin [ßHCG]), vital signs (blood pressure [BP], pulse, temperature), 12-lead electrocardiograms (ECGs), physical examinations, and assessment of concomitant medications (only part two of the study).

All subjects were required to have met the inclusion and exclusion criteria described below. However, minor deviations that were clinically insignificant and that posed no safety concerns had been deemed acceptable by the Investigator and the Sponsor, consistent with the protocol.

All subjects considered for study participation were required to have met the following inclusion criteria:
1. Subject was male or female.
2. Female subject and female partner of male subject:

Must not have been pregnant (female subjects must have had a negative urine pregnancy test prior to entry into the study).

Must not have been breast feeding.

Must not have been planning to become pregnant during the study period or within 3 months following the study.

Must have been adhering to an adequate form of contraception prior to entry into the study and for an additional 3 months after the follow-up visit.
3. Subject was aged between 18 and 45 years inclusive.
4. Subject had signed the informed consent form (ICF).
5. Subject's body mass index (BMI) was between 18.0 and 30.0 kg/m² inclusive.
6. Subject was considered to be in good health in the opinion of the Investigator by evaluating the safety assessments.
7. Subjects must have been able to communicate well with the Investigator, to understand and comply with the requirements of the study, and to understand and sign the written ICF. Subjects were excluded from the study if there was evidence of any of the following criteria:

Subject had a clinically significant illness in the 4 weeks before screening.

Females of childbearing potential and female partners of male subjects who had not used a safe contraceptive measure for 3 months prior to the study and were not willing to use a safe contraceptive measure for the duration of the study; examples of a safe contraceptive measure included intra-uterine device or oral contraceptives, diaphragm, or condom if used in combination with a spermicide.

Subject used a prescribed medication in the 2 weeks before dosing or used over-the-counter preparations (including vitamins and supplements) for 1 week before dosing (with the exception of paracetamol, which was allowed up to 48 hours before dosing), and hormonal contraceptives.

Subject used dietary supplements rich in omega-3 or omega-6 fatty acids.

Subject had a significant history of drug/solvent abuse or had positive test results at screening for DOA.

Subject had a history of alcohol abuse, in the opinion of the Investigator, or drank in excess of 28 units per week (males) or 21 units per week (females) at the time of screening.

Subject was, in the opinion of the Investigator, not suitable for participation in the study.

Subject had participated in another clinical study with a study drug/device within 3 months before the first day of administration of study drug.

Subject had a positive test result for HIV antibodies, Hepatitis B surface antigen, or Hepatitis C antibodies at screening.

Subject had a serious adverse reaction or significant hypersensitivity to any drug.

Subject had donated blood or blood products within 3 months before screening.

Subject had known hypersensitivity to any ingredients of the study drug.

Subjects were free to withdraw consent from the study at any time for any reason. In addition, the Investigator could withdraw a subject from study participation if, in the Investigator's opinion, it was in the best interest of the subject. A subject would be withdrawn from the study for any of the following reasons:

Withdrawal of consent any time

Deviations from the protocol

Incidental illness

An AE (adverse effect).

Although a subject was not obliged to give a reason for premature withdrawal, the Investigator was to make a reasonable effort to obtain the reason while fully respecting the subject's rights. If there was a medical reason for withdrawal of consent, the subject was to remain under the supervision of the Investigator until the subject was in satisfactory health; the Investigator was to conduct a follow-up assessment.

If the Investigator deemed it was considered in the best interest of the subject's welfare, the Investigator was to inform the subject's General Practitioner of the medical reason for the subject's withdrawal from the study. Every effort was to be made to contact a subject who failed to return to the site for any follow-up appointments in order to ensure that the subject was in satisfactory health.

In Part one of the study, subjects in Cohorts one to three were administered a single dose of study drug (either 500-, 1000-, or 2000-mg as 500-mg DS107G capsules or matching placebo capsules) in parallel on study Day 1 after at least an 8-hour fast, according to Table 20. Cohort 4 was initiated following the review of safety data from Cohort 3 by the SMC.

Food effect was evaluated in Cohort two at least 14 days after the first dose at which time a second single dose was administered. Subjects were administered a 1000-mg dose of study drug with 240 mL of water after a 10-hour fasting period and 30 minutes after starting consumption of a meal. Subjects then refrained from food intake for at least 4 hours after dosing. Food consumption was standardized for at least 12 hours after dosing using a standardized high-fat meal (800 to 1000 kcal with 500 to 600 kcal from fat and 250 kcal from carbohydrates). A typical standard test meal consisted of two eggs fried in butter, two strips of bacon, two slices of toast with butter, 120 mL of hash brown potatoes, and 240 mL of whole milk.

In Part two of the study (Cohorts five and six), subjects were administered study drug (500-mg DS107G capsules or matching placebo capsules) once daily in the fasted state for 28 days. Subjects in Cohort five received study drug first and, if they tolerated the 2000-mg daily dose for the first 14 days, subjects in Cohort six were started on the 4000-mg daily dose for 28 days.

TABLE 20
Treatment Cohorts

| | | | | |
|---|---|---|---|---|
| | | | Number | Fasted |
| | | | of | or Fed |
| Cohort | Subjects | Dose (mg) | capsules | State |

Part 1: Single Ascending Doses

| | | | | |
|---|---|---|---|---|
| 1 | 8 | 500-mg DGLA or placebo capsules | 1 | Fasted |
| 2 | 8 | 1000-mg DGLA or placebo capsules | 2 | Fasted |
| | 8 | 1000-mg DGLA or placebo capsules | 2 | Fed |
| 3 | 8 | 2000-mg DGLA or placebo capsules | 4 | Fasted |
| 4 | 8 | 4000-mg DGLA or placebo capsules | 8 | Fasted |

Part 2: Multiple Ascending Doses for 28 Consecutive Days

| | | | | |
|---|---|---|---|---|
| 5 | 8 | 2000-mg DGLA or placebo capsules | 4 | Fasted |
| 6 | 8 | 4000-mg DGLA or placebo capsules | 8 | Fasted |

Note:
Each DS107G capsule contained 500 mg DGLA.

Any escalation in the dose level or commencement of a subsequent cohort was decided by the Principal Investigator. A minimum of 5 subjects with evaluable safety data from Cohort three (2000-mg dose) was required before commencing Cohort four (4000-mg dose). In Part 2, a minimum of 5 subjects with 14 days of evaluable safety data from Cohort 5 (2000 mg/day) was required before commencing Cohort six (4000 mg/day).

One strength of DS107G DGLA capsule was developed containing 500 mg of DGLA free fatty acid (FFA). Capsules included the following excipients: DGLA FFA (stabilized with a nominal 2000 ppm dl-alpha tocopherol). All excipients in the capsule shell were commonly used in soft gelatin products and include a transmissible spongiform encephalopathy (TSE) certified gelatin shell containing the following ingredients: purified water, the plasticizer glycerol, the colorant titanium dioxide, and the processing aids lecithin and medium chain triglyceride. The placebo capsule (DS107G Placebo capsule) for clinical studies consisted of liquid paraffin encapsulated in a soft gelatin shell and was identical in appearance to the DGLA capsule.

Subjects meeting the eligibility criteria were randomly assigned to receive DGLA (500, 1000, 2000, 4000 mg doses) or matching placebo capsules using a randomization schedule. The randomization was block randomization with an active treatment to placebo ratio of 3:1. The randomization schedule was generated by Planimeter using SAS® 9.1.3 SP4.

Subjects were not permitted to use prescribed medication during the 2 weeks prior to dosing or to use over-the-counter preparations (including vitamins and supplements) and hormonal contraceptives for 1 week before dosing, with the exception of paracetamol, which was allowed up to 48 hours before dosing. In addition, subjects were not allowed to use dietary supplements rich in omega-3 or omega-6 fatty acids. Subjects were not permitted to consume alcohol in excess of 28 units per week (male subjects) or 21 units per week (female subjects).

Subjects were advised to avoid consuming food supplements rich in omega-3 or omega-6 fatty acids (e.g., cod liver oil capsules) for 4 weeks prior to Screening and during the study.

Subjects were advised to avoid eating poppy seeds and food containing poppy seeds for at least 24 hours before urine sample collection for testing DOA as poppy seeds can sometimes cause a positive test result.

Subjects in Cohort two undergoing evaluation of the food effect on oral 1000 mg DGLA capsules were restricted from food consumption for at least 4 hours after dosing. Food consumption was standardized for at least 12 hours post dose using a standardized high-fat meal.

Subjects were to refrain from taking any systemic or over-the-counter medication (including vitamins and supplements) during the study with the exception of hormonal contraceptives. Paracetamol (at a dose up to 4 g/day) was allowed up to 48 hours before dosing. Subjects were also to refrain from alcohol consumption from 48 hours prior to the first administration of study drug (Day 1) until the follow-up visit.

There were no restrictions on caffeine intake or tobacco use prior to or during the study. Subjects were required to avoid exercise and strenuous physical activity for at least 3 to 4 hours before the blood was collected for the clinical laboratory test.

Analysis of plasma concentrations of DGLA (free and total) and free 15-HETrE were performed in blood samples obtained from the subjects. In addition, skin blister fluids were obtained at days 1, 7, 14, and 28 for analysis of free and total DGLA.

Plasma concentrations of dihydroxytestosterone (DHT) were assessed in Part 2 as a biomarker or exploratory efficacy endpoint.

Analysis of all plasma and skin blister fluid samples was performed using validated methods. Concentrations of free and total DGLA were measured in plasma and skin blister fluid by liquid chromatography with tandem mass spectrometry (LC/MS/MS); the quantitation range was 100 to 10,000 ng/mL for free DGLA and 5000 to 500,000 ng/mL for total DGLA. Plasma concentrations of free 15-HETrE were measured by LC/MS/MS with a quantitation range of 100 to 10,000 ng/mL. Plasma concentrations of DHT were measured by LC/MS with a quantitation range of 0.02 to 1.5 ng/m L.

Subjects were monitored throughout their confinement at the Phase 1 unit for adverse reactions to the study drug and/or procedures.

The pharmacokinetic assessments used in this study were standard for evaluation of potential therapeutic agents. The safety assessments included methods that were considered to be standard for a Phase 1 clinical study.

The study was exploratory, and no formal power calculations were performed. The number of subjects planned for each cohort (8 subjects) was considered sufficient to allow assessment of the safety and systemic exposure of DS107G capsule.

Analysis of populations included the following:
The Intention-to-Treat (ITT) population consists of all randomized subjects who had been administered at least 1 dose of study drug.
The Per Protocol (PP) population consists of all subjects in the ITT population who had no major protocol violation, as defined in the SAP.
The PK population contains all subjects included in the PP population who had evaluable PK data derived from plasma. A plasma concentration observation was considered a valid, evaluable measurement if the following data were available: study identification number, randomization number, date and time of sampling, dose and concentration. A series of such measurements from the same sample were considered complete if each protocol prescribed concentration was evaluable. Plasma PK data were evaluable by definition if the data contained a complete series of observations. Any missing plasma PK observation would result in incomplete PK data, thus a subject with any missing plasma PK observation was excluded from the PK population. Subjects randomized to placebo were also excluded from the PK population.

The populations defined above were generated separately for study Part 1 and Part 2 data. Safety analysis was performed on the ITT population.

Safety analyses were performed on the safety population tabulated by treatment arm, cohort, and visit. All safety data were characterized by descriptive statistical tools. No hypothesis was set to investigate in the study protocol. Evaluation of safety assessments was performed in a descriptive manner. Continuous variables were characterized by their mean, standard deviation (SD), median, minimum and maximum values; discrete variables were characterized by their absolute (frequency) and relative (percentage) distribution.

Primary endpoints (PK characteristics derived from single and multiple oral doses of DS107G capsule) were derived with the help of non-compartmental PK modeling. Secondary endpoints consisted of characterizing the effect of food on the PK of a single oral dose of DS107G capsule and characterizing the PK of DGLA in human skin following multiple oral doses of DS107G capsule.

The secondary endpoints were reported using the following descriptive analytical tools: number of valid observations, mean, standard deviation (SD), median, minimum, and maximum values derived for continuous parameters grouped by treatment arm, visit and cohort.

No formal hypothesis testing was performed during evaluation of study data. In Part 1 (single ascending dose), the extent of exposure was 1 day by definition (in case of successful administration of the study drug). In Part 2 (multiple ascending doses), the extent of exposure was calculated as the day of last study drug intake minus the day of first study drug intake+1 (when no interruption in study drug administration was documented). In case of interruption(s) the result of the above formula was decreased with the number of interruption(s).

Statistical analysis of the PK data was performed using SAS Software (Version 9.1.3). No pharmacodynamics analysis was performed.

Plasma PK parameters of DGLA and of 15-HETrE were estimated using model-independent techniques (non-compartmental analysis) and included: Cₘₐₓ, tₘₐₓ, Clast, Tlast, AUC₀₋₂₄, AUC_{0-inf}, AUC_{last, λz}, CL, V, and t1/2 for Parts 1 and 2, and tₘᵢₙ, cmin, CLss, Vss, C_{avg}, and % PTF for only Part 2 (steady-state) data.

All PK parameters were summarized descriptively, no formal statistical testing was performed due to the exploratory nature of the study.

In the single-dose setting (Part 1), study drug discontinuation was not a potential outcome in Cohorts one, three and four; therefore, no reporting activity was required for these cohorts. For Cohort 2, the enrolled subjects received study drug twice: a single dose administered in the fasted state and a second single dose administered in the fed state.

The following amendments were also made to the protocol.
Table of Clinical Studies added, to clarify previous clinical studies with oral DGLA and their safety conclusions.
Risk/benefit assessment added, to clarify the assessed risks and benefits of the proposed trial for the benefit of the Investigator and Ethics Committee.
Enrollment criteria were changed, such that subjects who were enrolled in Part 1 of the study (single-dose cohorts) could be brought back and re-enrolled in Part 2 (multiple-dose cohorts), provided they had no AEs related to study drug and they had a washout period of at least 14 days before starting the multiple-dose regimen. The rationale for this change was to aid recruitment without compromising the safety of the volunteers.
Clinical laboratory tests for coagulation (prothrombin time and activated partial prothrombin time [APTT]) were added as assessments at all clinical laboratory assessment timepoints in Part 2 of the study (multiple-dose Cohorts 5 and 6). These assessments were added to monitor any potential changes in clotting factors as an exploratory biomarker for future studies.
Ambiguous wording was replaced to clarify that single ECG recordings would be obtained.
15-HETrE was removed as an analyte for testing in Part 2 (multiple-dose cohorts), as preliminary PK data from Part 1 cohorts revealed that all samples for 15-HETrE were below the limit of quantification (BLQ).
In addition to free (unesterified) DGLA, the quantification of “total” DGLA was added to all analyses, as the plasma PK profile of the “total” may differ from the “free.”
Also, changes to the planned analyses were included to provide additional analyses and statistical output.

Forty subjects were screened; of those 40, 4 subjects were excluded for not fulfilling inclusion/exclusion requirements, and 4 subjects withdrew consent. Disposition of the 32 subjects randomized to study drug is presented in Table 21.

TABLE 21
Summary of Subject Disposition - ITT population (Single Dose).

| | Dose Level of DS107G | | | | | |
|---|---|---|---|---|---|---|
| | 500 mg | 1000 mg | 2000 mg | 4000 mg | Placebo¹ | Overall |
| | (N = 6) | (N = 6) | (N = 6) | (N = 6) | (N = 8) | (N = 32) |
| | n (%) | n (%) | n (%) | n (%) | n (%) | n (%) |
| Total number of subjects | 6 (100) | 6 (100) | 6 (100) | 6 (100) | 8 (100) | 32 (100) |
| Randomized | 6 (100) | 6 (100) | 6 (100) | 6 (100) | 8 (100) | 32 (100) |
| Treated | 6 (100) | 6 (100) | 6 (100) | 6 (100) | 8 (100) | 32 (100) |
| Completed | 6 (100) | 6 (100) | 6 (100) | 6 (100) | 8 (100) | 32 (100) |
| Premature discontinuation | 0 | 0 | 0 | 0 | 0 | 0 |

ITT = intent-to-treat
¹Of the 8 subjects who received placebo, 2 subjects were randomized to each of the DS107G dosing cohorts.

Plasma concentrations of free and total DGLA at baseline, before administration of DS107G, were summarized in Table 22. These concentrations were generally highly variable.

TABLE 22
Baseline Plasma Concentrations of DGLA (Single Dose, Pharmacokinetic Population)

| | | Dose Level of DS107G | | | | |
|---|---|---|---|---|---|---|
| | | 500 mg | 1000 mg | | 2000 mg | 4000 mg |
| Analyte | | Fasted | Fasted | Fed | Fasted | Fasted |
| (unit) | | (n = 5) | (n = 6) | (n = 6) | (n = 6) | (n = 6) |
| Free DGLA | Mean | 237.0 | 302.3 | 262.3 | 172.8 | 378.2 |
| (ng/mL) | (SD) | (76.95) | (29.80) | (70.22) | (29.67) | (101.18) |
| | Min, Max | 150, 357 | 268, 347 | 133, 327 | 134, 204 | 241, 538 |
| Total DGLA | Mean | 37740.0 | 40583.3 | 42550.0 | 33083.3 | 29900.0 |
| (ng/mL) | (SD) | (14371.60) | (13664.47) | (16206.02) | (8247.52) | (10164.05) |
| | Min, Max | 22000, 57100 | 26700, 63900 | 28400, 66600 | 22700, 43200 | 19100, 45500 |

DGLA = dihomo-gamma-linolenic acid;
Max = maximum;
mean = arithmetic mean;
min = minimum;
N = number of subjects providing data;
SD = standard deviation

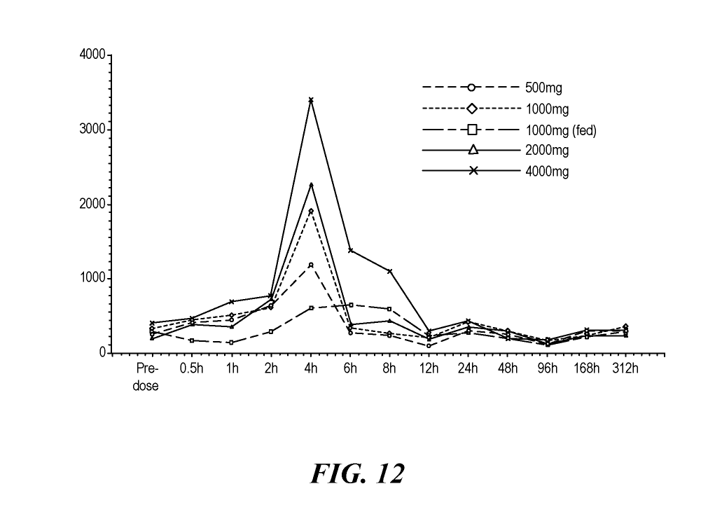

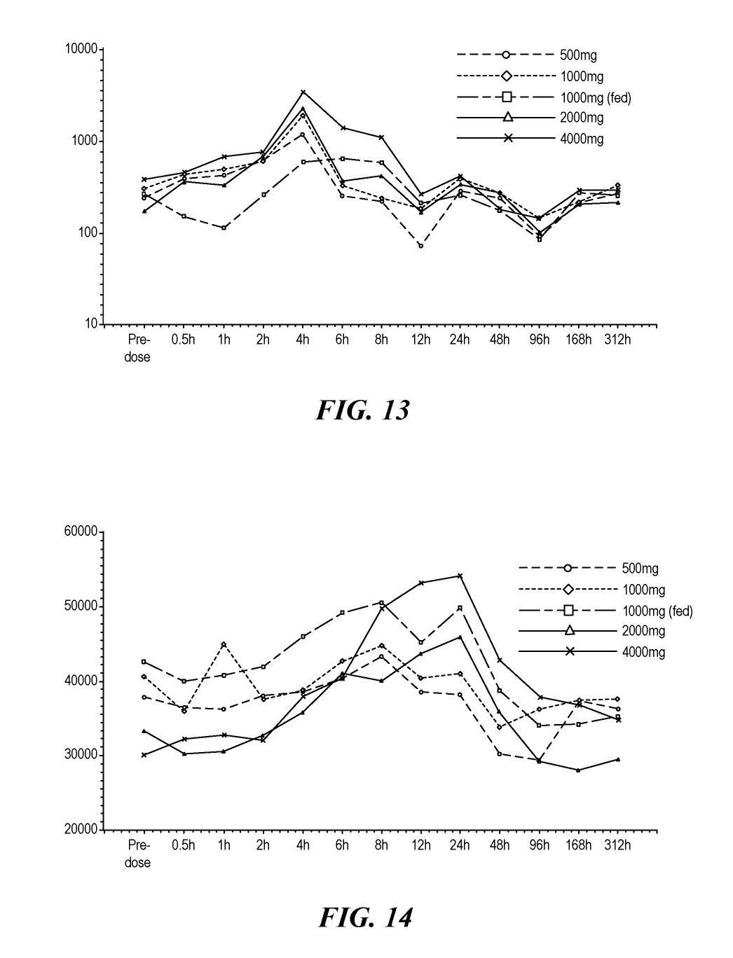

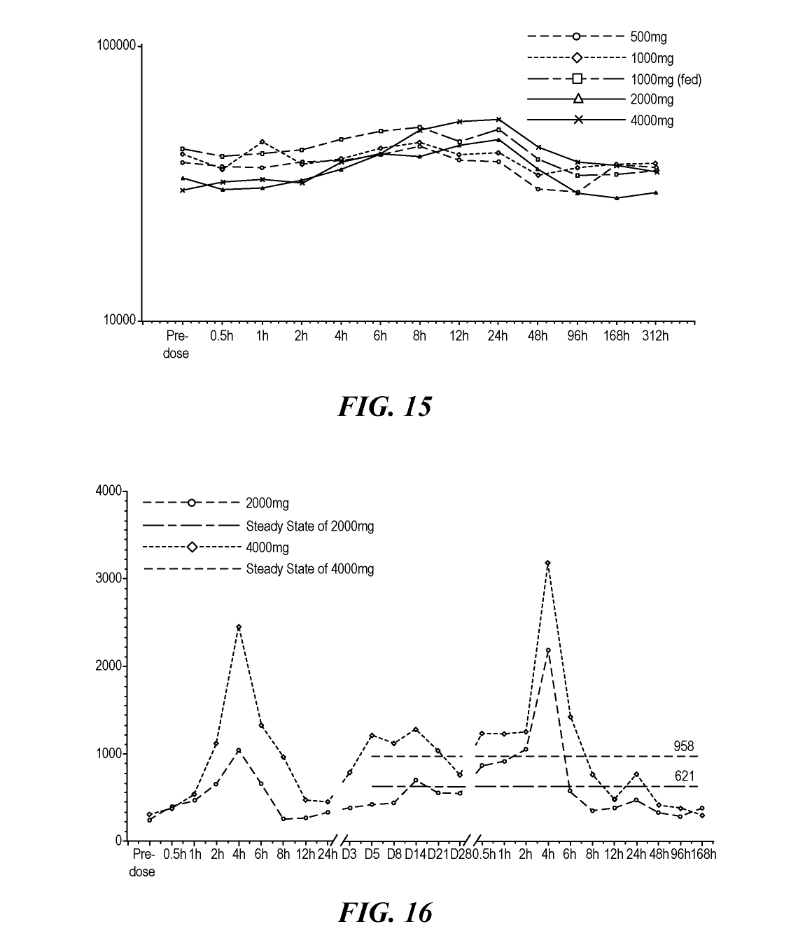

Mean plasma concentrations after a single dose of DS107G are shown graphically by dose cohort for free DGLA in FIG. 12 (linear plot) and FIG. 13 (log-linear plot), and for total DGLA in FIG. 14 (linear plot) and FIG. 15 (log-linear plot).

After a single dose of DS107G capsules (500, 1000, 2000, and 4000 mg), inter-subject variability (measured by SD) of free and total DGLA was high for both plasma concentrations and baseline-corrected PK parameters. Under fasted conditions, mean baseline-corrected Cₘₐₓ and AUC0-24 for both free and total DGLA increased in a linear manner (Table 23, Table 24). The median time to maximum concentration (Tₘₐₓ) was 4 hours for free DGLA (Table 23) and not consistent across the dose, with values of 8 hours at the 2 lower doses and 18 hours at the 2 higher doses (Table 24). Although baseline-corrected elimination PK parameters could be determined for less than half the subjects in some cohorts, there was no evidence of nonlinear pharmacokinetics in elimination half-life or clearance for either free or total DGLA (Table 24 and Table 25). Although not evaluated statistically, administration of a single 1000-mg dose of DS107G under fasted conditions resulted in an approximately 50% higher rate and extent of total DGLA absorption based on baseline corrected mean Cₘₐₓ and AUC₀₋₂₄ (Table 22).

TABLE 23
Baseline-corrected Plasma Pharmacokinetic Parameters for Free DGLA after
Fasted Administration (Single Dose, Pharmacokinetic Population)

| | | Dose Level of DS107G | | | |
|---|---|---|---|---|---|
| | | 500 mg | 1000 mg | 2000 mg | 4000 mg |
| Parameter (unit) | Statistic | (n = 5) | (n = 6) | (n = 6) | (n = 6) |
| tₘₐₓ (h) | N | 5 | 6 | 6 | 6 |
| | Median | 4.00 | 4.00 | 4.00 | 4.00 |
| | Min, Max | 4.0, 4.0 | 4.0, 4.0 | 4.0, 8.0 | 4.0, 6.0 |
| Cₘₐₓ (ng/mL) | N | 5 | 6 | 6 | 6 |
| | Mean | 934.6 | 1602.8 | 2074.0 | 3021.8 |
| | SD | 776.87 | 1202.43 | 1276.03 | 1581.97 |
| Cₘₐₓ/Dose (1/kL) | N | 5 | 6 | 6 | 6 |
| | Mean | 1.869 | 1.603 | 1.037 | 0.755 |
| | SD | 1.5537 | 1.2024 | 0.6380 | 0.3955 |
| AUC₀₋₂₄ (ng · h/mL) | N | 5 | 6 | 6 | 6 |
| | Mean | 2728.1 | 3998.5 | 6429.0 | 9436.8 |
| | SD | 2253.49 | 1885.27 | 2119.39 | 2227.25 |
| AUC₀₋₂₄/Dose (h/kL) | N | 5 | 6 | 6 | 6 |
| | Mean | 5.46 | 4.00 | 3.21 | 2.36 |
| | SD | 4.507 | 1.885 | 1.060 | 0.557 |
| λ_{z} (1/h) | N | 2 | 5 | 5 | 5 |
| | Mean | 0.0035 | 0.0487 | 0.0200 | 0.0603 |
| | SD | 0.00101 | 0.05133 | 0.02445 | 0.10642 |
| t_{1/2} (h) | N | 2 | 5 | 5 | 5 |
| | Median | 206.33 | 28.30 | 57.33 | 67.53 |
| | Min, Max | 164.3, 248.4 | 6.2, 3226.3 | 11.0, 181.1 | 2.8, 80.9 |
| AUC_{0-inf} (ng · h/mL) | N | 2 | 5 | 5 | 5 |
| | Mean | 21483.3 | 218955.6 | 26877.0 | 17113.6 |
| | SD | 14902.37 | 474291.75 | 19394.67 | 3545.72 |
| AUC_{0-inf}/Dose (h/kL) | N | 2 | 5 | 5 | 5 |
| | Mean | 42.97 | 218.96 | 13.44 | 4.28 |
| | SD | 29.805 | 474.292 | 9.697 | 0.886 |
| CL/F (kL/h) | N | 2 | 5 | 5 | 5 |
| | Mean | 0.0306 | 0.1866 | 0.1339 | 0.2420 |
| | SD | 0.02126 | 0.18154 | 0.11952 | 0.05066 |
| Vz/F (kL) | N | 2 | 5 | 5 | 5 |
| | Mean | 10.035 | 7.356 | 8.977 | 17.460 |
| | SD | 8.9574 | 9.1148 | 4.0610 | 11.5516 |

Max = maximum;
mean = arithmetic mean;
min = minimum;
N = number of subjects providing data;
SD = standard deviation
Note:
Predose DGLA concentration was subtracted from subsequent concentrations before parameter calculation; negative values were replaced by zero.

TABLE 24
Baseline-corrected Plasma Pharmacokinetic Parameters for Total DGLA
after Fasted Administration (Single Dose, Pharmacokinetic Population)

| | | Dose Level of DS107G | | | |
|---|---|---|---|---|---|
| | | 500 mg | 1000 mg | 2000 mg | 4000 mg |
| Parameter (unit) | | (n = 5) | (n = 6) | (n = 6) | (n = 6) |
| tₘₐₓ (h) | N | 5 | 6 | 6 | 6 |
| | Median | 8.00 | 8.00 | 18.00 | 18.00 |
| | Min, Max | 6.0, 312.0 | 0.0, 312.0 | 6.0, 24.0 | 8.0, 24.0 |
| Cₘₐₓ (ng/mL) | N | 5 | 6 | 6 | 6 |
| | Mean | 7200.0 | 15750.0 | 17733.3 | 25883.3 |
| | SD | 2801.79 | 16326.02 | 9867.66 | 13658.32 |
| Cₘₐₓ/Dose (1/kL) | N | 5 | 6 | 6 | 6 |
| | Mean | 14.400 | 15.750 | 8.867 | 6.471 |
| | SD | 5.6036 | 16.3260 | 4.9338 | 3.4146 |
| AUC₀₋₂₄ (ng · h/mL) | N | 5 | 4 | 6 | 6 |
| | Mean | 39556.6 | 215369.7 | 210508.9 | 437754.0 |
| | SD | 37969.05 | 346249.37 | 167660.31 | 278838.30 |
| AUC₀₋₂₄/Dose (h/kL) | N | 5 | 4 | 6 | 6 |
| | Mean | 79.11 | 215.37 | 105.25 | 109.44 |
| | SD | 75.938 | 346.249 | 83.830 | 69.710 |
| λ_{z} (1/h) | N | 2 | 2 | 2 | 5 |
| | Mean | 0.0013 | 0.0136 | 0.0084 | 0.0102 |
| | SD | 0.00042 | 0.01381 | 0.00301 | 0.00993 |
| t_{1/2} (h) | N | 2 | 2 | 2 | 5 |
| | Median | 552.07 | 105.42 | 88.04 | 71.03 |
| | Min, Max | 428.3, 675.9 | 29.7, 181.2 | 65.8, 110.3 | 26.7, 464.7 |
| AUC_{0-inf} (ng · h/mL) | N | 2 | 2 | 2 | 5 |
| | Mean | 4415966.6 | 4031410.9 | 2276495.1 | 5973518.8 |
| | SD | 723917.02 | 5304874.41 | 1948487.62 | 7261476.90 |
| AUC_{0-inf}/Dose (h/kL) | N | 2 | 2 | 2 | 5 |
| | Mean | 8831.93 | 4031.41 | 1138.25 | 1493.38 |
| | SD | 1447.834 | 5304.874 | 974.244 | 1815.369 |
| CL/F (kL/h) | N | 2 | 2 | 2 | 5 |
| | Mean | 0.0001 | 0.0018 | 0.0014 | 0.0036 |
| | SD | 0.00002 | 0.00243 | 0.00119 | 0.00519 |
| Vz/F (kL) | N | 2 | 2 | 2 | 5 |
| | Mean | 0.089 | 0.093 | 0.203 | 0.275 |
| | SD | 0.0140 | 0.0843 | 0.2136 | 0.1569 |

Max = maximum;
mean = arithmetic mean;
min = minimum;
N = number of subjects providing data;
SD = standard deviation
Note:
Predose DGLA concentration was subtracted from subsequent concentrations before parameter calculation; negative values were replaced by zero.

The effect of food on the single-dose baseline-corrected PK of free and total DGLA using the 1000-mg dose (Cohort two) was evaluated and reported in Table 26. Briefly, the mean baseline-corrected free DGLA Cₘₐₓ was about 3 fold higher and occurred 1 hour (median) sooner under fasted conditions than under fed conditions (Table 26). Mean free DGLA baseline-corrected AUC0-24 under fasted conditions was about 2 fold higher than the value under fed conditions. Thus an increased rate and extent of DGLA absorption under fasted conditions was observed. There were no clear differences for fasted vs fed conditions in elimination of free DGLA (Table 25).

For total DGLA, mean baseline-corrected Cₘₐₓ was about 1.5-fold higher and tₘₐₓ occurred about 50% sooner (median, 8 vs 15 h) under fasted conditions than under fed conditions (Table 25). Mean baseline-corrected AUC₀₋₂₄ under fasted conditions was about 1.8-fold higher than under fed conditions. Just under half of the subjects (2/6 fasted, 3/6 fed) had sufficient data to estimate total DGLA λz, t1/2, clearance, and volume of distribution. These data suggest an increased rate and extend of total DGLA absorption under fasted condition. No reliable conclusion regarding total DGLA elimination or volume distribution due to small data population.

Based on the PK parameters shown in Table 25, mean baseline-corrected Cₘₐₓ was ˜10 fold (fasted) and ˜20-fold (fed) higher for total DGLA than for free DGLA. Mean baseline-corrected AUC₀₋₂₄ was ˜54-fold (fasted) and ˜56-fold (fed) higher for total DGLA than for free DGLA.

TABLE 25
Baseline-corrected Plasma Pharmacokinetic Parameters for DGLA, After
Fasted vs Fed Administration (Single Dose, Pharmacokinetic Population)

| | | 1000 mg DS107G | | | |
|---|---|---|---|---|---|
| | | Free DGLA | | Total DGLA | |
| | | Fasted | Fed | Fasted | Fed |
| Parameter (unit) | Statistic | (n = 6) | (n = 6) | (n = 6) | (n = 6) |
| tₘₐₓ (h) | N | 6 | 6 | 6 | 6 |
| | Median | 4.00 | 5.00 | 8.00 | 15.00 |
| | Min, Max | 4.0, 4.0 | 4.0, 8.0 | 0.0, 312.0 | 6.0, 24.0 |
| Cₘₐₓ (ng/mL) | N | 6 | 6 | 6 | 6 |
| | Mean | 1602.8 | 520.2 | 15750.0 | 10316.7 |
| | SD | 1202.43 | 235.54 | 16326.02 | 2043.93 |
| Cₘₐₓ/Dose (1/kL) | N | 6 | 6 | 6 | 6 |
| | Mean | 1.603 | 0.520 | 15.750 | 10.317 |
| | SD | 1.2024 | 0.2355 | 16.3260 | 2.0439 |
| AUC₀₋₂₄ (ng · h/mL) | N | 6 | 6 | 4 | 6 |
| | Mean | 3998.5 | 2102.7 | 215369.7 | 117389.7 |
| | SD | 1885.27 | 1174.23 | 346249.37 | 70263.85 |
| AUC_{0-24/}Dose (h/kL) | N | 6 | 6 | 4 | 6 |
| | Mean | 4.00 | 2.10 | 215.37 | 117.39 |
| | SD | 1.885 | 1.174 | 346.249 | 70.264 |
| λ_{z} (1/h) | N | 5 | 3 | 2 | 3 |
| | Mean | 0.0487 | 0.0403 | 0.0136 | 0.0805 |
| | SD | 0.05133 | 0.06154 | 0.01381 | 0.06155 |
| t_{1/2} (h) | N | 5 | 3 | 2 | 3 |
| | Median | 28.30 | 140.86 | 105.42 | 6.62 |
| | Min, Max | 6.2, 3226.3 | 6.2, 149.4 | 29.7, 181.2 | 5.5, 66.0 |
| AUC_{0-inf} (ng · h/mL) | N | 5 | 3 | 2 | 3 |
| | Mean | 218955.6 | 29150.8 | 4031410.9 | 267053.2 |
| | SD | 474291.75 | 23323.42 | 5304874.41 | 335666.87 |
| AUC_{0-inf}/Dose (h/kL) | N | 5 | 3 | 2 | 3 |
| | Mean | 218.96 | 29.15 | 4031.41 | 267.05 |
| | SD | 474.292 | 23.323 | 5304.874 | 335.667 |
| CL/F (kL/h) | N | 5 | 3 | 2 | 3 |
| | Mean | 0.1866 | 0.1658 | 0.0018 | 0.0096 |
| | SD | 0.18154 | 0.24651 | 0.00243 | 0.00702 |
| Vz/F (kL) | N | 5 | 3 | 2 | 3 |
| | Mean | 7.356 | 4.623 | 0.093 | 0.128 |
| | SD | 9.1148 | 0.5166 | 0.0843 | 0.0209 |

Max = maximum;
mean = arithmetic mean;
min = minimum;
N = number of subjects providing data;
SD = standard deviation
Note:
Predose DGLA concentration was subtracted from subsequent concentrations before parameter calculation; negative values were replaced by zero.

DSI07G was well tolerated as a single dose at amounts 500, 1000, 2000, or 4000 mg to healthy volunteers. The most common TEAE reported were mild to moderate diarrhea (reported term: loose stool) by a similar percentage of subjects between the active-treatment and placebo-control subjects (incidence: 5/24 [20.8%] active-treated subjects vs 2/8 placebo-control subjects). The diarrhea events were of relatively short duration, and all (including those occurring in placebo-control subjects) were considered by the Investigator to be possibly related to study drug. Of note, subjects who received a second single dose of DS107G and who had TEAEs of diarrhea in the fasted state did not have a recurrence of diarrhea. All other TEAEs occurred in only 1 subject each and only in the active-treatment groups, including: mild infection, oropharyngeal pain and pharyngitis; and moderately severe pyrexia and urinary tract infection after dosing in the fed state.

### Multiple-Dose Results—Study Part 2

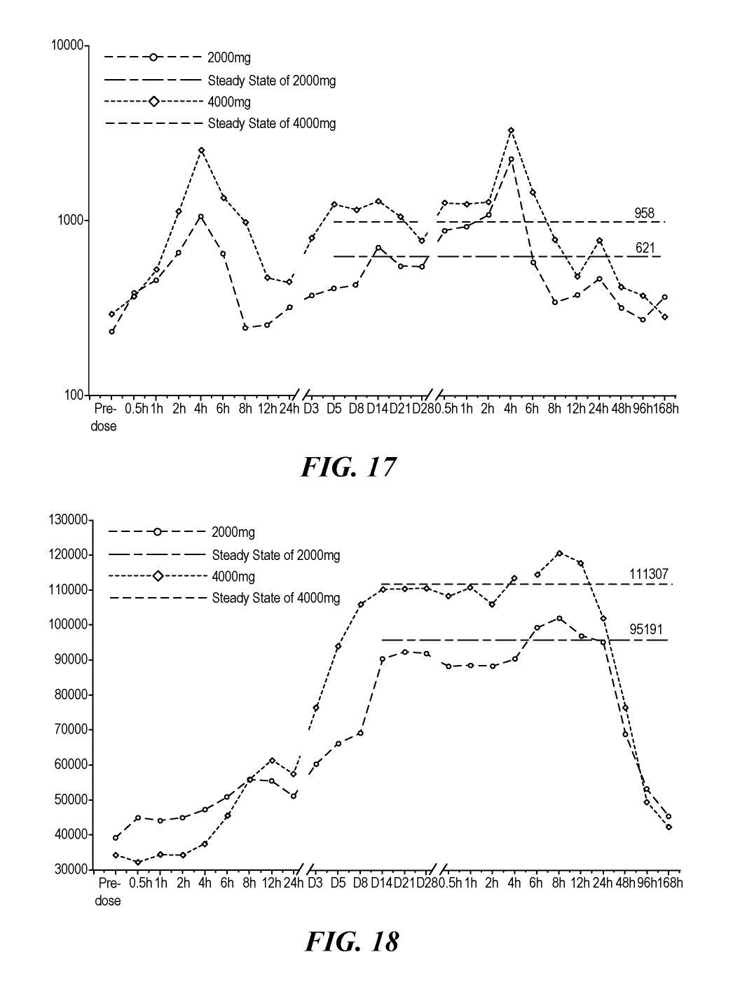

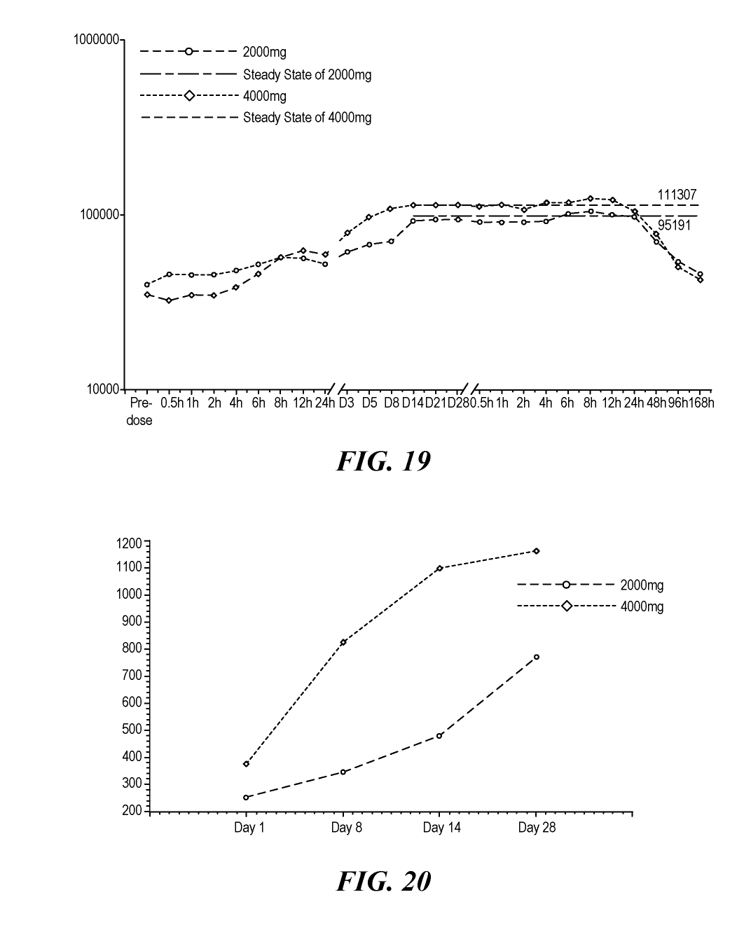

Mean plasma concentrations and the average concentration at steady-state for Part 2 are shown graphically by dose cohort for free DGLA in FIG. 16 (linear plot) and FIG. 17 (log-linear plot), and for total DGLA in FIG. 18 (linear plot) and FIG. 19 (log-linear plot). On Days 1 and 28, mean concentrations of free DGLA peaked at about 4 hours after dosing, whereas no mean peak concentration was evident for total DGLA. Mean concentrations of both free and total DGLA increased over time with repeated dosing, though the increase for total DGLA was more pronounced. Plasma concentrations appeared to reach steady-state by around Day 14 for both doses (2000 and 4000 mg daily) and analytes (free and total DGLA) based on visual inspection of the mean concentration plots. When the dose doubled from 2000 to 4000 mg daily, the average concentration at steady state increased 1.6-fold for free DGLA but only 1.2-fold for total DGLA, suggesting one or more saturable processes at the higher dose.

PK parameter was computed after correcting the dosed DGLA concentrations with baseline DGLA concentrations.

The plasma baseline corrected pharmacokinetics for free DGLA is reported in Table 26. Briefly, mean free DGLA baseline-corrected Cₘₐₓ and AUC were higher in the higher DS107G dose cohort on both days evaluated. Mean baseline-corrected Cₘₐₓ for the 4000 mg dose was ˜3-fold higher than for the 2000 mg dose on Day 1 but only ˜1.4 fold higher on Day 28. Mean baseline-corrected AUC0-24 for the 4000 mg dose was ˜2.5 fold higher than for the 2000 mg dose on Day 1 and only ˜1.7-fold higher on Day 28. The changes with dose were linear for baseline-corrected Cₘₐₓ and AUC0-24 on Day 1, but only for baseline-corrected AUC0-24 on Day 28. High inter-subject variability might have caused this inconsistency. Median tₘₐₓ was similar for both dose cohorts on Day 1 and Day 28, with values of 4 or 5 hours. Median elimination t1/2 was longer on Day 28 than on Day 1, with the value on Day 28 dependent on the time interval evaluated. Mean clearance decreased and mean volume of distribution increased with multiple doses.

TABLE 26
Plasma Baseline-corrected Pharmacokinetic Parameters for
Free DGLA (Multiple-dose, Pharmacokinetic Population)

| | | Dose Level of DS107G | | | | | |
|---|---|---|---|---|---|---|---|
| | | Day 1 | | Day 28 | | Day 28 | |
| | | 0 to 24 hours | | 0 to 24 hours | | 0 to 168 hours | |
| Parameter | | 2000 mg | 4000 mg | 2000 mg | 4000 mg | 2000 mg | 4000 mg |
| (unit) | Statistic | (n = 6) | (n = 6) | (n = 6) | (n = 6) | (n = 6) | (n = 6) |
| tₘₐₓ | N | 6 | 6 | 6 | 6 | 6 | 6 |
| (h) | Median | 4.00 | 5.00 | 4.00 | 4.00 | 4.00 | 4.00 |
| | Min, Max | 4.0, 6.0 | 2.0, 8.0 | 2.0, 4.0 | 4.0, 4.0 | 2.0, 4.0 | 4.0, 4.0 |
| Cₘₐₓ | N | 6 | 6 | 6 | 6 | 6 | 6 |
| (ng/mL) | Mean | 850.5 | 2641.5 | 1999.3 | 2873.2 | 1999.3 | 2873.2 |
| | SD | 458.84 | 1734.46 | 1003.19 | 1246.97 | 1003.19 | 1246.97 |
| Cₘₐₓ/Dose | N | 6 | 6 | 6 | 6 | 6 | 6 |
| (1/kL) | Mean | 0.425 | 0.660 | 1.000 | 0.718 | 1.000 | 0.718 |
| | SD | 0.2294 | 0.4336 | 0.5016 | 0.3117 | 0.5016 | 0.3117 |
| AUC₀₋₂₄ | N | 6 | 6 | 6 | 6 | 6 | 6 |
| (ng · h/mL) | Mean | 4548.7 | 11441.4 | 9248.1 | 16155.2 | 9248.1 | 16155.2 |
| | SD | 3330.51 | 5313.29 | 2559.31 | 6367.97 | 2559.31 | 6367.97 |
| AUC₀₋₂₄/Dose | N | 6 | 6 | 6 | 6 | 6 | 6 |
| (h/kL) | Mean | 2.27 | 2.86 | 4.62 | 4.04 | 4.62 | 4.04 |
| | SD | 1.665 | 1.328 | 1.280 | 1.592 | 1.280 | 1.592 |
| λ_{z} | N | 2 | 6 | 3 | 5 | 5 | 6 |
| (1/h) | Mean | 0.2133 | 0.1504 | 0.1282 | 0.0596 | 0.0250 | 0.0404 |
| | SD | 0.16812 | 0.10843 | 0.15619 | 0.02019 | 0.02983 | 0.05960 |
| t_{1/2} | N | 2 | 6 | 3 | 5 | 5 | 6 |
| (h) | Median | 4.71 | 5.92 | 14.04 | 12.72 | 63.59 | 48.18 |
| | Min, Max | 2.1, 7.3 | 2.0, 18.3 | 2.2, 25.6 | 7.5, 16.5 | 9.0, 132.1 | 4.3, 65.7 |
| AUC_{0-inf} | N | 2 | 6 | 3 | 5 | 5 | 6 |
| (ng · h/mL) | Mean | 5955.6 | 13900.3 | 10389.0 | 22926.3 | 43024.8 | 43184.3 |
| | SD | 5216.07 | 4993.97 | 2677.43 | 6485.37 | 35641.74 | 21443.83 |
| AUC_{0-inf}/Dose | N | 2 | 6 | 3 | 5 | 5 | 6 |
| (h/kL) | Mean | 2.98 | 3.48 | 5.19 | 5.73 | 21.51 | 10.80 |
| | SD | 2.608 | 1.248 | 1.339 | 1.621 | 17.821 | 5.361 |
| CL/F | N | 2 | 6 | 3 | 5 | 5 | 6 |
| (kL/h) | Mean | 0.5448 | 0.3325 | 0.2002 | 0.1865 | 0.0949 | 0.1266 |
| | SD | 0.47711 | 0.16310 | 0.04493 | 0.05432 | 0.08636 | 0.08671 |
| Vz/F | N | 2 | 6 | 3 | 5 | 5 | 6 |
| (kL) | Mean | 2.426 | 3.063 | 3.592 | 3.493 | 4.470 | 5.273 |
| | SD | 0.3245 | 1.9716 | 2.5139 | 1.5255 | 1.5179 | 2.1568 |

Max = maximum;
mean = arithmetic mean;
min = minimum;
N = number of subjects providing data;
SD = standard deviation
Note:
Predose DGLA concentration was subtracted from subsequent concentrations before parameter calculation; negative values were replaced by zero.

The steady state plasma baseline corrected pharmacokinetics for free DGLA is reported in Table 27. Briefly, the plasma concentrations of free and total DGLA increased with repeated dosing, and achieved steady-state at approximately Day 14. When at steady state, evaluated on Day 28 (0-24 hours), Peak Trough Fluctuation (PTF) was quite high for both dose cohorts (mean, ˜430% and ˜490%). The mean accumulation ratio (AR) was greater for the 2000 mg than the 4000 mg dose cohort for both Cₘₐₓ and AUC (ARs of ˜2.8 and ˜3.3 for 2000 mg vs ˜1.4 and ˜1.6 for 4000 mg. The data suggest saturable kinetics and/or presence of change in the distribution volume with repeated dosing of free DGLA.

TABLE 27
Steady-state Plasma Baseline-corrected Pharmacokinetic Parameters
for Free DGLA (Multiple-dose, Pharmacokinetic Population)

| | | Day 28: 0 to 24 hours | |
|---|---|---|---|
| | | Dose Level of DS107G | |
| Parameter | | 2000 mg | 4000 mg |
| (unit) | | (n = 6) | (n = 6) |
| tₘᵢₙ | N | 6 | 6 |
| (h) | Median | 8.00 | 8.00 |
| | Min, Max | 6.0, 24.0 | 0.0, 12.0 |
| Cₘᵢₙ | N | 6 | 6 |
| (ng/mL) | Mean | 100.8 | 147.0 |
| | SD | 103.34 | 230.37 |
| CLₛₛ/F | N | 6 | 6 |
| (kL/h) | Mean | 0.2295 | 0.2858 |
| | SD | 0.05874 | 0.12493 |
| Vzₛₛ/F | N | 3 | 5 |
| (kL) | Mean | 5.3096 | 5.7563 |
| | SD | 4.02532 | 3.12495 |
| C_{avg} | N | 6 | 6 |
| (ng/mL) | Mean | 385.3 | 673.1 |
| | SD | 106.64 | 265.33 |
| % PTF | N | 6 | 6 |
| (%) | Mean | 492.54 | 431.97 |
| | SD | 212.957 | 207.312 |
| AR(Cₘₐₓ) | N | 6 | 6 |
| | Mean | 2.796 | 1.385 |
| | SD | 1.7385 | 0.7726 |
| AR(AUC) | N | 6 | 6 |
| | Mean | 3.340 | 1.561 |
| | SD | 2.4958 | 0.6377 |

AR = accumulation ratio;
Max = maximum;
mean = arithmetic mean;
min = minimum;
N = number of subjects providing data;
PTF = peak trough fluctuation;
SD = standard deviation
Note:
Predose DGLA concentration was subtracted from subsequent concentrations before parameter calculation; negative values were replaced by zero.

The plasma baseline corrected pharmacokinetics for total DGLA is reported in Table 28. Briefly, mean total DGLA baseline-corrected Cₘₐₓ and AUC₀₋₂₄ were higher in the higher DS107G dose cohort on both days evaluated, as expected. Mean baseline-corrected Cₘₐₓ and AUC₀₋₂₄ for the 4000 mg dose were ˜1.5- and ˜1.5-fold higher, respectively, than for the 2000 mg dose on Day 1 but only ˜1.2- and ˜1.4-fold higher than for the 2000 mg dose on Day 28.

The changes with dose in baseline-corrected Cₘₐₓ and AUC4 were not linear for total DGLA on either day evaluated. High inter-subject variability might have caused this inconsistency. Median Tₘₐₓ occurred sooner with multiple doses (8-10 h) than with a single dose (10-18 h) in both dose cohorts. Median total DGLA elimination t_{1/2} for the 2000 mg dose cohort was 34.4 to 44.0 hours when assessed over 24 hours (Days 1 and 28, respectively), and 62.6 hours on Day 28 when evaluated over 0 to 168 hours. Mean clearance and volume of distribution decreased with multiple doses.

TABLE 28
Plasma Baseline-corrected Pharmacokinetic Parameters for Total DGLA (Multiple-dose, Pharmacokinetic Population)

| | | Dose Level of DS107G | | | | | |
|---|---|---|---|---|---|---|---|
| | | Day 1 | | Day 28 | | Day 28 | |
| | | 0 to 24 hours | | 0 to 24 hours | | 0 to 168 hours | |
| Parameter | | 2000 mg | 4000 mg | 2000 mg | 4000 mg | 2000 mg | 4000 mg |
| (unit) | Statistic | (n = 6) | (n = 6) | (n = 6) | (n = 6) | (n = 6) | (n = 6) |
| tₘₐₓ | N | 6 | 6 | 6 | 6 | 6 | 6 |
| (h) | Median | 10.00 | 18.00 | 8.00 | 10.00 | 8.00 | 10.0 |
| | Min, Max | 8.0, 12.0 | 12.0, 24.0 | 6.0, 24.0 | 8.0, 24.0 | 6.0, 24.0 | 8.0, 24.0 |
| Cₘₐₓ | N | 6 | 6 | 6 | 6 | 6 | 6 |
| (ng/mL) | Mean | 18100.0 | 27866.7 | 75583.3 | 90866.7 | 75583.3 | 90866.7 |
| | SD | 15113.44 | 10391.66 | 30385.55 | 33000.16 | 30385.55 | 33000.16 |
| Cₘₐₓ/Dose | N | 6 | 6 | 6 | 6 | 6 | 6 |
| (1/kL) | Mean | 9.050 | 6.967 | 37.792 | 22.717 | 37.792 | 22.717 |
| | SD | 7.5567 | 2.5979 | 15.1928 | 8.2500 | 15.1928 | 8.2500 |
| AUC₀₋₂₄ | N | 6 | 6 | 6 | 6 | 6 | 6 |
| (ng · h/mL) | Mean | 298184.2 | 456199.5 | 1351372.5 | 1860574.3 | 1351372.5 | 1860574.3 |
| | SD | 336969.41 | 151586.31 | 379053.12 | 760386.93 | 379053.12 | 760386.93 |
| AUC₀₋₂₄/Dose | N | 6 | 6 | 6 | 6 | 6 | 6 |
| (h/kL) | Mean | 149.09 | 114.05 | 675.69 | 465.14 | 675.69 | 465.14 |
| | SD | 168.485 | 37.897 | 189.527 | 190.097 | 189.527 | 190.097 |
| λ_{z} | N | 3 | 0 | 4 | 3 | 6 | 6 |
| (1/h) | Mean | 0.221 | | 0.0199 | 0.0471 | 0.0169 | 0.0194 |
| | SD | 0.02180 | | 0.00966 | 0.05409 | 0.01064 | 0.00927 |
| t_{1/2} | N | 3 | 0 | 4 | 3 | 6 | 6 |
| (h) | Median | 44.04 | | 34.40 | 36.57 | 62.64 | 39.21 |
| | Min, Max | 14.9, 162.5 | | 22.2, 89.2 | 6.3, 53.8 | 20.0, 74.9 | 19.7, 70.9 |
| AUC_{0-inf} | N | 3 | 0 | 4 | 3 | 6 | 6 |
| (ng · h/mL) | Mean | 4161212.0 | | 5159152.1 | 4779645.9 | 5027822.8 | 6303903.1 |
| | SD | 6203131.92 | | 4269186.29 | 2974257.92 | 2286762.41 | 4913744.09 |
| AUC_{0-inf}/Dose | N | 3 | 0 | 4 | 3 | 6 | 6 |
| (h/kL) | Mean | 2080.61 | | 2579.58 | 1194.91 | 2513.91 | 1575.98 |
| | SD | 3101.566 | | 2134.593 | 743.564 | 1143.381 | 1228.436 |
| CL/F | N | 3 | 0 | 4 | 3 | 6 | 6 |
| (kL/h) | Mean | 0.0027 | | 0.0006 | 0.0012 | 0.0005 | 0.0009 |
| | SD | 0.00266 | | 0.00041 | 0.00099 | 0.00027 | 0.00056 |
| Vz/F | N | 3 | 0 | 4 | 3 | 6 | 6 |
| (kL) | Mean | 0.106 | | 0.029 | 0.035 | 0.032 | 0.046 |
| | SD | 0.0597 | | 0.0095 | 0.0114 | 0.0133 | 0.0133 |

Max = maximum;
mean = arithmetic mean;
min = minimum;
N = number of subjects providing data;
SD = standard deviation
Note:
Predose DGLA concentration was subtracted from subsequent concentrations before parameter calculation; negative values were replaced by zero.

The steady state plasma baseline corrected pharmacokinetics for free DGLA is reported in Table 29. Briefly, at steady-state, evaluated on Day 28 (0-24 hours), Peak Trough Fluctuation (PTF) was quite high for both dose cohorts (mean, 62.5% and 44.9%). The mean AR was greater for the 2000 mg than the 4000 mg dose cohort for both Cₘₐₓ and AUC. The data suggest saturable kinetics and/or presence of change in the distribution volume with repeated dosing of total DGLA.

TABLE 29
Steady-state Plasma Baseline-corrected Pharmacokinetic Parameters for
Total DGLA (Multiple-dose, Pharmacokinetic Population)

| | | Day 28: 0 to 24 hours | |
|---|---|---|---|
| | | Dose Level of DS107G | |
| Parameter | | 2000 mg | 4000 mg |
| (unit) | Statistic | (n = 6) | (n = 6) |
| tₘᵢₙ | N | 6 | 6 |
| (h) | Median | 1.50 | 2.00 |
| | Min, Max | 0.0, 6.0 | 2.0, 24.0 |
| Cₘᵢₙ | N | 6 | 6 |
| (ng/mL) | Mean | 37933.3 | 59216.7 |
| | SD | 10890.12 | 37002.29 |
| CLₛₛ/F | N | 6 | 6 |
| (kL/h) | Mean | 0.0016 | 0.0025 |
| | SD | 0.00051 | 0.00107 |
| Vzₛₛ/F | N | 4 | 3 |
| (kL) | Mean | 0.0980 | 0.1047 |
| | SD | 0.04161 | 0.07339 |
| C_{avg} | N | 6 | 6 |
| (ng/mL) | Mean | 56307.2 | 77523.9 |
| | SD | 15793.88 | 31682.79 |
| % PTF | N | 6 | 6 |
| (%) | Mean | 62.50 | 44.90 |
| | SD | 31.403 | 38.967 |
| AR(Cₘₐₓ) | N | 6 | 6 |
| | Mean | 5.156 | 3.440 |
| | SD | 2.2542 | 1.2090 |
| AR(AUC) | N | 6 | 6 |
| | Mean | 7.991 | 4.163 |
| | SD | 5.9537 | 1.2331 |

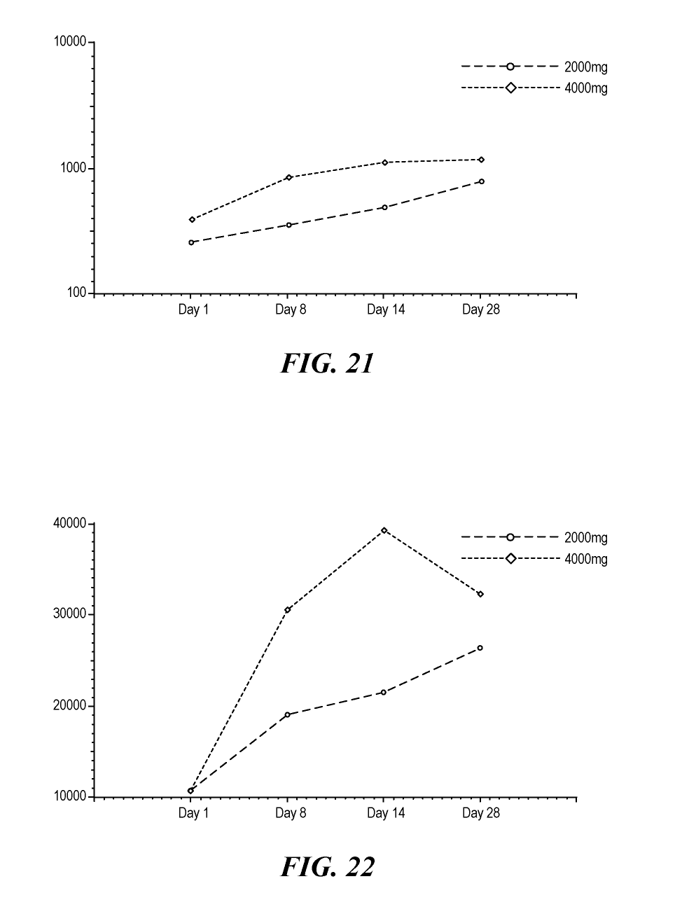

Mean free DGLA concentrations in skin blister fluid are shown by dose cohort in FIG. 20 (linear plot) and FIG. 21 (log-linear plot). Mean concentrations approximately doubled with a doubling in dose (based on concentrations from Days 1, 8, 14, and 28), and accumulated with repeated doses in both regimens. Mean free DGLA concentrations on Day 28 were about 3-fold higher than those on Day 1 for both 2000 and 4000 mg daily.

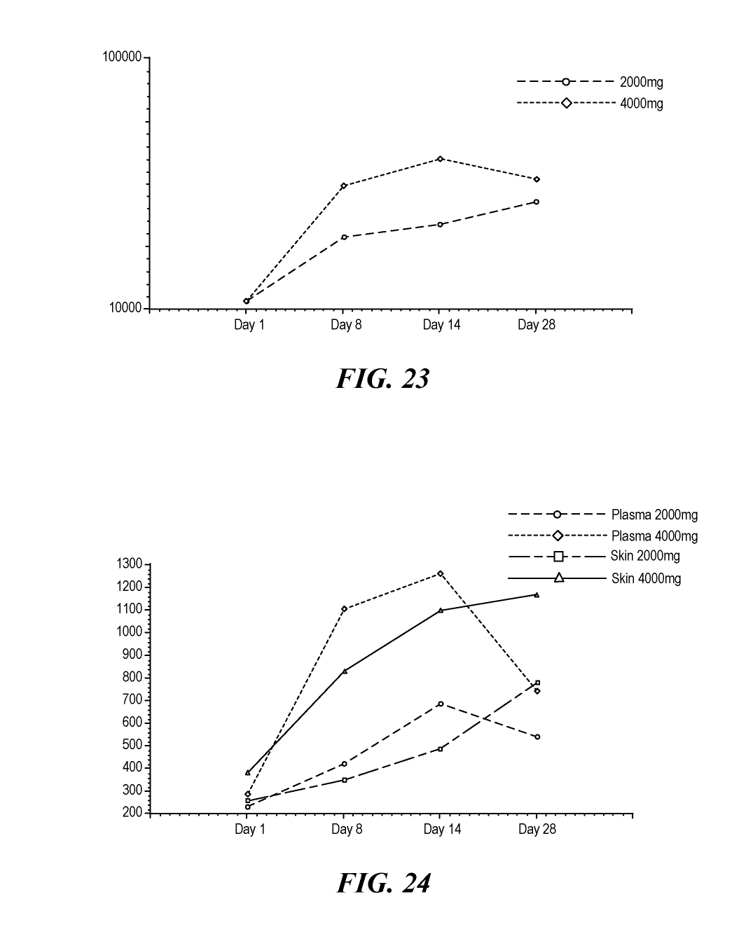

Mean skin blister fluid concentration of total DGLA (ng/mL, linear plot) by dose cohort (Multiple-dose, PK Population) is shown in FIG. 22. Mean skin blister fluid concentration of total DGLA (ng/mL, log-linear plot) by dose cohort (Multiple-dose, PK Population) is shown in FIG. 23.

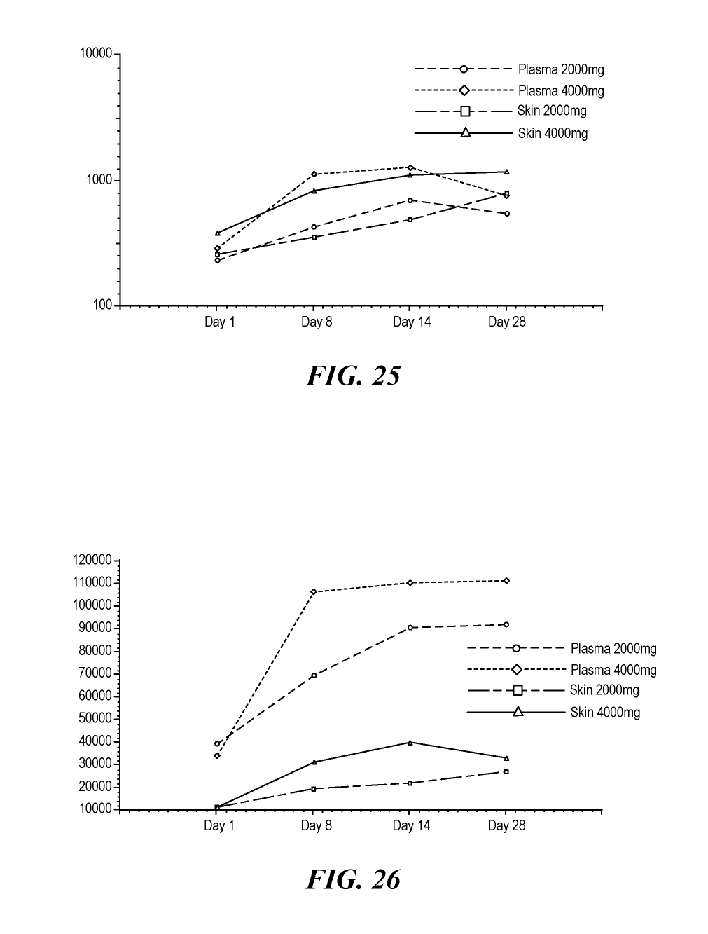

Mean total DGLA concentrations in skin blister fluid are shown by dose cohort in FIG. 24 (linear plot) and FIG. 25 (log-linear plot). Mean concentrations of total DGLA increased about 1.4-fold with a doubling in dose (based on concentrations from Days 1, 8, 14, and 28). Mean total DGLA concentrations on Day 28 were about 2.5- and 3-fold higher than those on Day 1 for 2000 and 4000 mg daily, respectively.

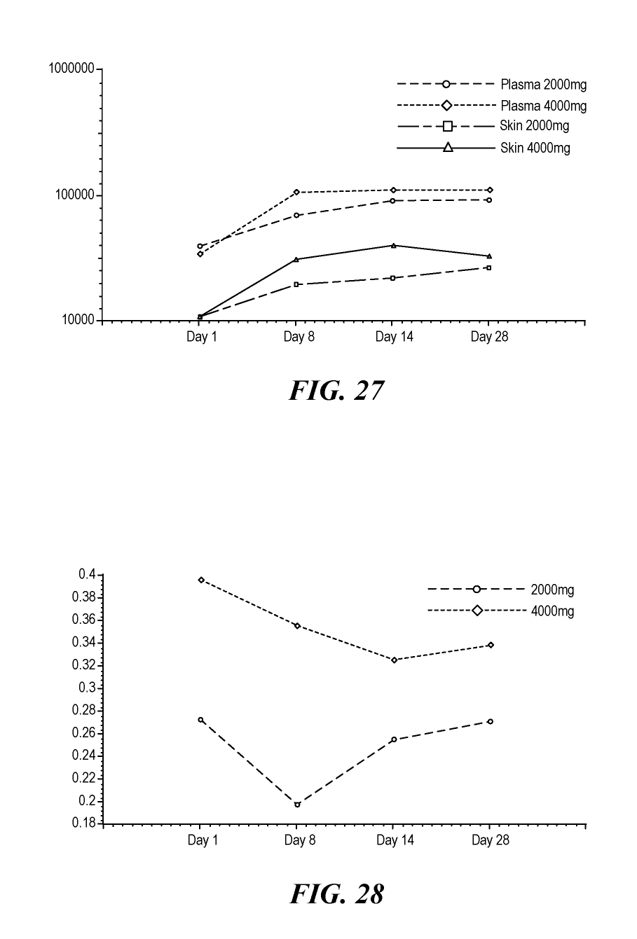

The concentration profiles in plasma and skin blister fluid were overlaid and mean concentrations of free DGLA were somewhat similar in plasma and skin blister fluid for the same DS107G dose at Day 8 and Day 14 (but not Day 28). FIG. 26 [linear plot] and FIG. 27 [log-linear plot]), suggest that DGLA distributes into plasma and skin similarly. For total DGLA, mean concentrations were much higher in plasma than in skin blister fluid for the same DS107G dose after Day 1 (FIG. 26 [linear plot] and FIG. 27 [log-linear plot]), indicating that total DGLA is found more readily in plasma than in skin. The mechanism for limited distribution of total DGLA into skin is most likely related to the lower quantity of lipids in the skin compared with plasma.

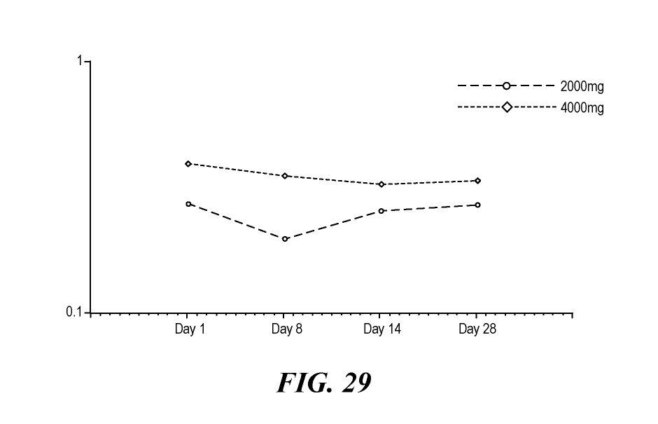

Plasma dihydrotestosterone (DHT) concentrations were quantified as an exploratory efficacy endpoint or biomarker. Inter-subject variability in the concentration data was high based on the SD at most time points. Mean plasma concentrations of DHT are shown by dose cohort in FIG. 28 (linear) and FIG. 29 (log-linear).

None of the samples had measurable free 15-HETrE concentrations; all concentrations were below the LLOQ (100 ng/mL).

There were no deaths for the multi-dose study. When administered to healthy volunteers as 2000- or 4000-mg doses once daily for 28 consecutive days, DSI07G was tolerated well, with the worst TEAE being mild to moderate diarrhea (reported term: loose stool) of relatively short duration. A majority of the events of diarrhea (incidence 7/12 [43.8%] active-treated subjects vs 0/4 [0.0%] placebo-control subjects) were considered by the Investigator to be possibly related to study drug. A higher proportion of subjects reported diarrhea in the 4000-mg group ( 4/6 [66.7%]) than in the 2000-mg group ( 3/6 [50%]). The incidence of TEAEs among active-treated subjects was much higher than that of the placebo-control subjects ( 11/12 [91.73%] subjects reporting a total of 52 TEAEs vs ¼ [25.0%] subjects reporting a total of 1 TEAE, respectively). There were no severe TEAEs, and other than TEAEs of diarrhea, all events were considered to be either not or unlikely related to study drug. Nausea was the next most commonly reported TEAE (10 events among 4/6 [66.7%] subjects in the 4000-mg treatment group); 9/10 of the events of nausea were mild and the other was moderate in severity. Other than diarrhea, all remaining TEAEs were reported in 2 subjects each (bronchitis and nasopharyngitis) or 1 subject each (abdominal pain, asthenia, pyrexia, blood CPK increased, CRP increased, WBC count decreased, dizziness, headache, cough, and haematoma), the majority of which were considered by the Investigator to be unlikely or not related to study drug. Other TEAEs considered to be possibly related to study drug were abdominal pain and asthenia (Reported Term “weakness”), both of which had temporal associations with events of loose stool.

No clinically significant abnormalities in vital signs or ECG observed in any patients in multiple dose study.

### Example 5

Further to the randomized, double-blind, placebo-controlled, phase II study of Example 2 above, a post hoc stratification analysis was performed using the results of that study to assess the efficacy of orally administered DS107G (DGLA) to patients with moderate to severe atopic dermatitis having normal eosinophil levels of less than about 0.3×10⁹/L compared to the same population receiving placebo and/or to the total study population. The primary objective of the post hoc stratification study was to compare the efficacy of orally administered DS107G capsules, versus placebo, in the treatment of adult patients with moderate to severe atopic dermatitis having normal eosinophil levels of less than about 0.3×10⁹/L. The secondary objective was to compare the efficacy of orally administered DS107G capsules in the treatment of adult patients with moderate to severe atopic dermatitis having normal eosinophil levels of less than about 0.3×10⁹/L versus the total study population. Unless stated otherwise, all elements of the post hoc stratification analysis were the same as those of the phase II study described in Example 2 above.

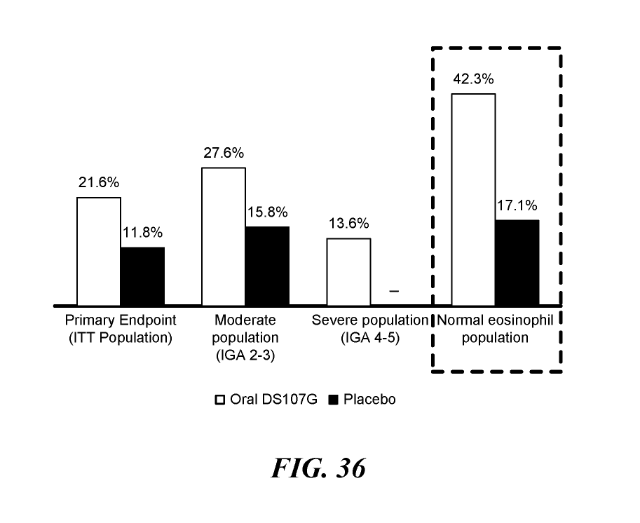

Results of the post hoc stratification analysis are shown in Tables 30-39 and FIGS. 36-43B. Based on the FDA standard primary efficacy end point for atopic dermatitis clinical trials (a two point drop in the Investigator Global Assessment (IGA), as well as an end of treatment IGA of 0 (clear) or 1 (almost clear)), the overall results from post hoc stratification analysis of the randomized, double-blind, placebo-controlled, phase II study of Example 2 above showed a greater effect of DS107 in patients having normal eosinophil levels compared either to placebo or the total study population. With respect to this study, patients having normal eosinophil levels did not have an active infection or any concomitant allergic diseases. More specifically, with respect to the subgroup of study patients having normal eosinophil levels, the number of IGA responders receiving DS107 increased to 42% from 22% of the same group receiving placebo. Similarly, for the subgroup of study patients having normal eosinophil levels, the pruritus reduction for patients receiving DS107 increased to 53% from 41% of the same group receiving placebo. In particular, this post hoc stratification analysis showed that 60% of the total study population of 102 patients had normal eosinophil levels (e.g., less than about 0.3×10⁹/L). Conversely, patients having high eosinophil levels (e.g., greater than about 0.3×10⁹/L) had a 0% IGA responder rate.

An overview of the total study population prior to the post hoc stratification analysis is listed below in Table 30 for DGLA and placebo.

TABLE 30
Total Study Population Receiving D5107 (“Active”)
or Placebo Before Post Hoc Stratification.
Total Study Population

| | |
|---|---|
| Treatment duration | 8 weeks |
| Route of administration | Oral |
| Patient population | Moderate to severe |
| Treatment regimen | Once daily |

Baseline Demographics

| | Active | Placebo |
|---|---|---|
| Total Patient Pop. (n) | 51 | 51 |

IGA at Baseline

| | | |
|---|---|---|
| 3 at baseline | 57% | 74% |
| 4 at baseline | 39% | 26% |
| 5 at baseline | 4% | — |
| Mean EASI Score | 18.7 | 16.4 |

Results

| | | |
|---|---|---|
| IGA responders (%) | 22% | 12% |
| EASI (% decrease) | 46% | 45% |
| SCORAD (% decrease) | 36% | 33% |
| Pruritus score decrease | c. (2.8) | c. (2.2) |
| (as measured by VAS) | | |
| Pruritus delta | 41% | 26% |
| (as measured by VAS) | | |
| Number of AEs (n) | 87 | 44 |
| Number of SAEs (n) | — | — |

The population of subjects having normal eosinophil levels of less than 0.3×10⁹/L was statistically isolated from the total study population. Table 31 shows data from the study population having normal eosinophil levels after post hoc stratification analysis for patients receiving DGLA and placebo.

TABLE 31
Study Population Having Normal Eosinophil Levels Receiving
D5107 (“Active”) or Placebo After Post Hoc Stratification.
Study Population Having Normal Eosinophil Levels

| | |
|---|---|
| Treatment duration | 8 weeks |
| Route of administration | Oral |
| Patient population | Moderate to severe |
| Treatment regimen | Once daily |

Baseline Demographics

| | Active | Placebo |
|---|---|---|
| Total Patient Pop. (n) | 26 | 35 |
| IGA at Baseline | | |
| 3 at baseline | 69% | 89% |
| 4 at baseline | 31% | 11% |
| 5 at baseline | — | — |
| Mean EASI Score | 14.4 | 14.1 |
| | Results | |
| IGA responders (%) | 42% | 17% |
| EASI (% decrease) | 51% | 30% |
| SCORAD (% decrease) | 44% | 29% |
| Pruritus score decrease | c. (2.0) | c. (1.5) |
| (as measured by VAS) | | |
| Pruritus delta | 53% | 27% |
| (as measured by VAS) | 29 | 27 |
| Number of AEs (n) | — | — |

A comparison of the total study population (see column “DS107 Phase 2a I.T.T.”), patients having normal eosinophil levels, and patients having high eosinophil levels separated by patients who received DS107 or placebo is shown in Table 32 below.

TABLE 32
Summary of Post Hoc Stratification Analysis

| | | | DS107 Phase | | DS107 Phase | |
|---|---|---|---|---|---|---|
| | DS107 | | 2a Normal | | 2a High | |
| | Phase 2a | | Eosinophil < | | Eosinophil > | |
| | I.T.T. | | 0.3 × 10⁹/L | | 0.3 × 10⁹/L | |
| Treatment | DS107 | Placebo | DS107 | Placebo | DS107 | Placebo |
| Dosing Regime | Daily | Daily | Daily | Daily | Daily | Daily |

| | | | |
|---|---|---|---|
| Duration | 8 weeks | 8 weeks | 8 weeks |

Baseline

| | | | | | | |
|---|---|---|---|---|---|---|
| Total Patient Population (n) | 51 | 51 | 26 | 35 | 24 | 16 |
| Mean EASI | 18.7 | 16.4 | 14.4 | 14.1 | 23.2 | 21.8 |

Results

| | | | | | | |
|---|---|---|---|---|---|---|
| IGA responder (%) | 21% | 12% | 42% * | 17% * | 0% | 0% |
| EASI Percentage Change | −46% | −45% | −51% * | −30% * | −20.1% | −26.4% |
| from Baseline | | | | | | |
| Pruritus Percentage Change | −41% | −26% | −53% * | −27% * | −6.5% | +9.4% |
| from Baseline | | | | | | |
| BSA Change from Baseline | −38% | −34% | −41% * | −22% * | −14.9% | −22.9% |
| EASI-50 (%) | 57% | 58% | 60% | 59% | 22.7% | 21.4% |
| EASI-75 (%) | 37% | 36% | 40% | 37% | 9.1% | 14.3% |

* last observation carried forward (LOCF)

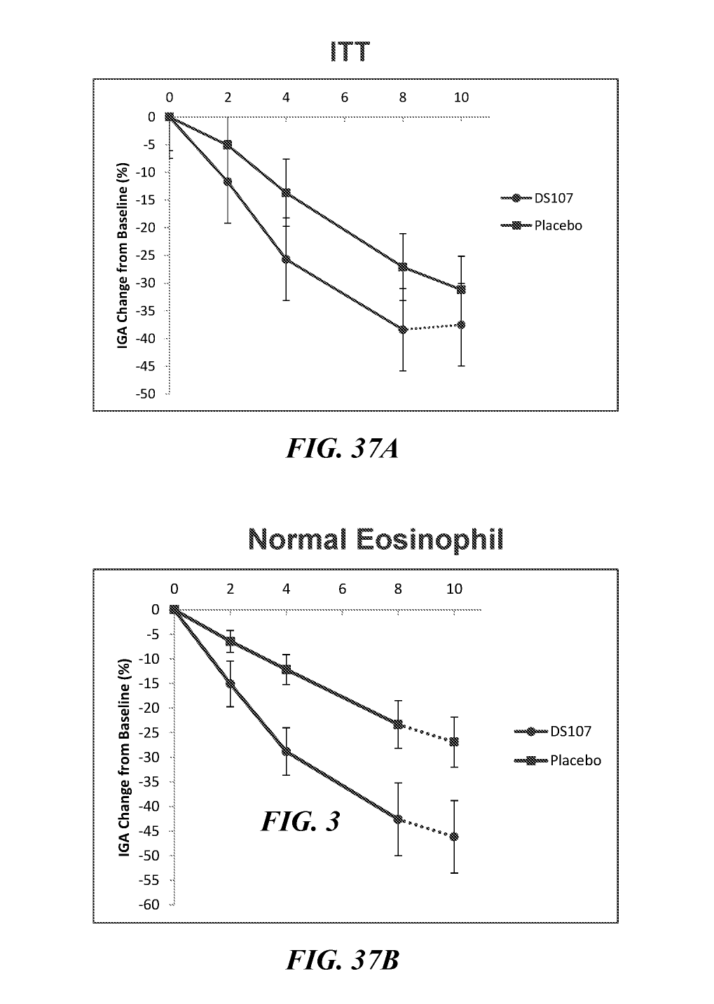

As shown in FIGS. 36-37B, study patients having normal eosinophil levels receiving DS107 had a greater change in IGA scores, relative to baseline, at weeks 2, 4, 6, 8 and 10 of the study, compared to the same group receiving placebo or the total study population. The corresponding data is shown in Tables 32 and 33.

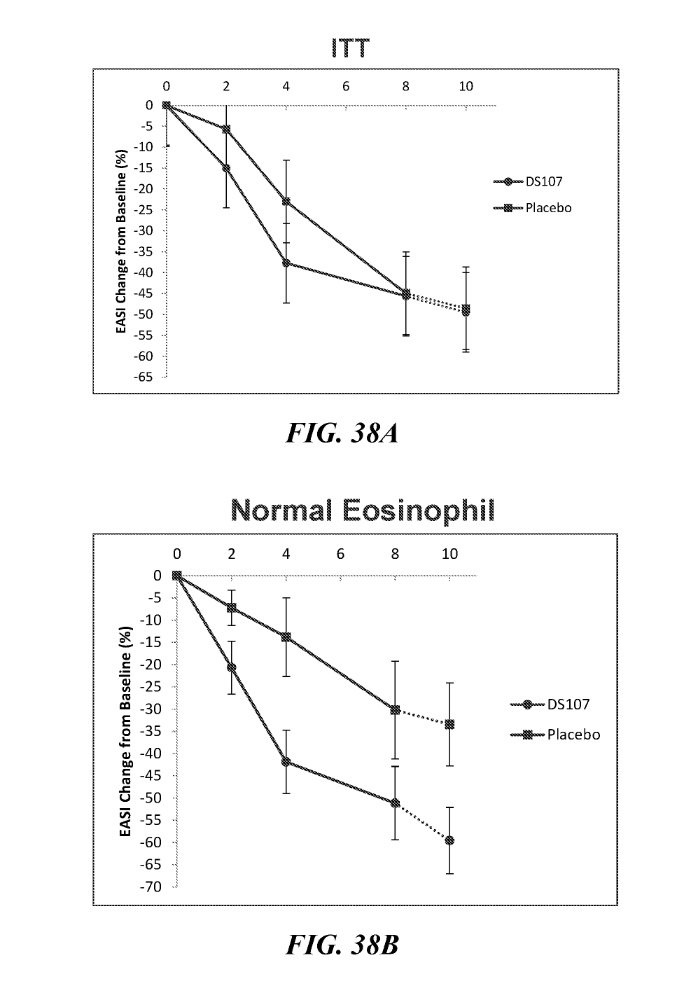

As shown in FIGS. 38A and 38B, study patients having normal eosinophil levels receiving DS107 had a greater change in EASI scores, relative to baseline, at weeks 2, 4, 6, 8 and 10 of the study, compared to the same group receiving placebo or the total study population. The corresponding data is shown in Table 34.

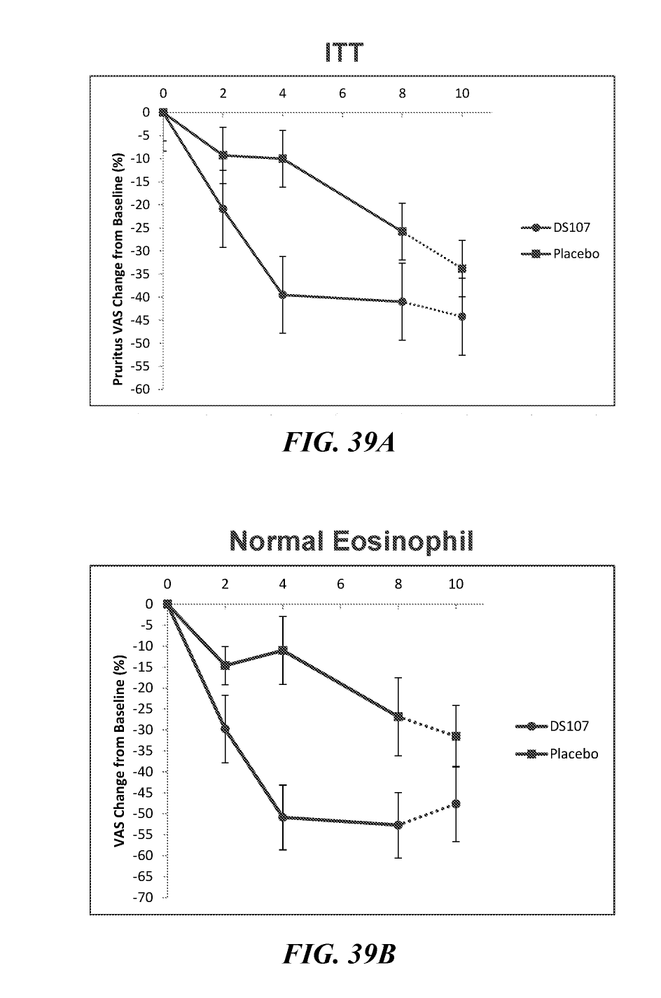

As shown in FIGS. 39A and 39B, study patients having normal eosinophil levels receiving DS107 had a greater change in VAS scores, relative to baseline, at weeks 2, 4, 6, 8 and 10 of the study, compared to the same group receiving placebo or the total study population. The corresponding data is shown in Table 35.

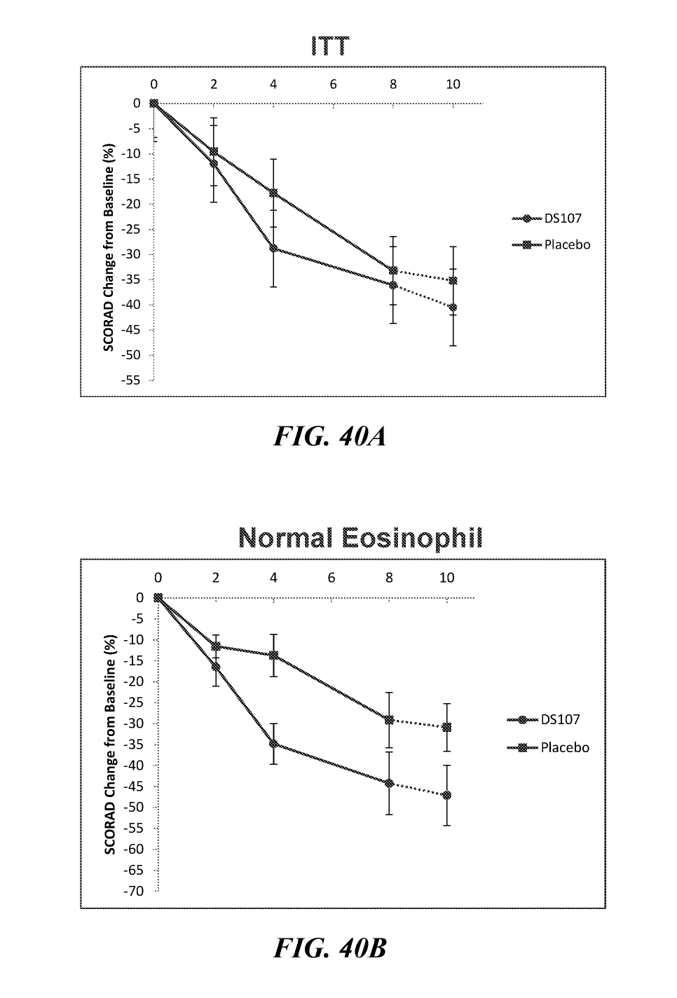

As shown in FIGS. 40A and 40B, study patients having normal eosinophil levels receiving DS107 had a greater change in SCORAD levels, relative to baseline, at weeks 2, 4, 6, 8 and 10 of the study, compared to the same group receiving placebo or the total study population. The corresponding data is shown in Table 36.

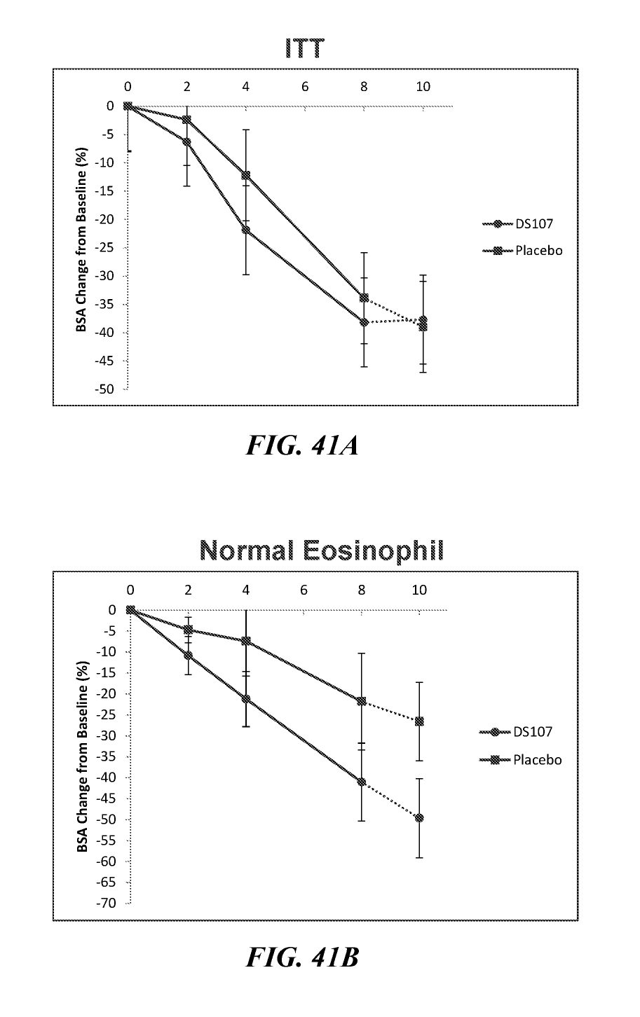

As shown in FIGS. 41A and 41B, study patients having normal eosinophil levels receiving DS107 had a greater change in BSA levels, relative to baseline, at weeks 2, 4, 6, 8 and 10 of the study, compared to the same group receiving placebo or the total study population. The corresponding data is shown in Table 37.

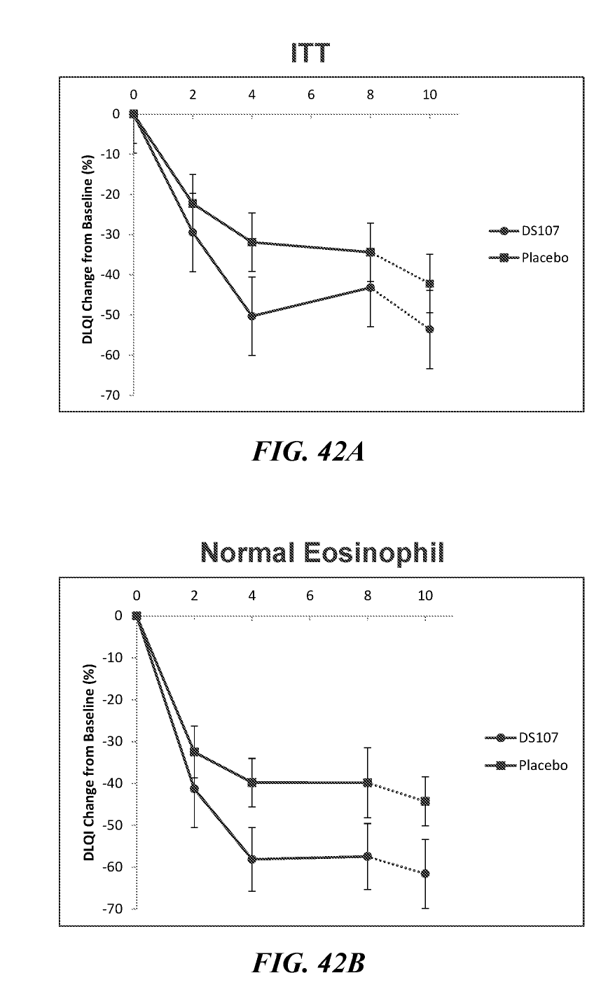

As shown in FIGS. 42A and 42B, study patients having normal eosinophil levels receiving DS107 had a greater change in DLQI levels, relative to baseline, at weeks 2, 4, 6, 8 and 10 of the study, compared to the same group receiving placebo or the total study population. The corresponding data is shown in Table 38.

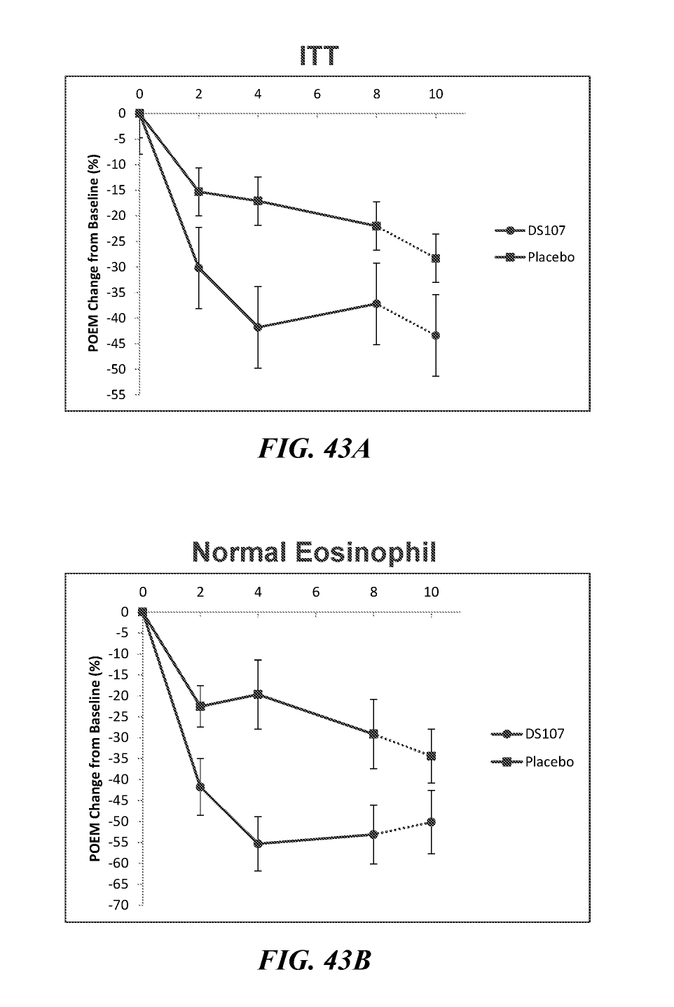

As shown in FIGS. 43A and 43B, study patients having normal eosinophil levels receiving DS107 had a greater change in POEM scores, relative to baseline, at weeks 2, 4, 6, 8 and 10 of the study, compared to the same group receiving placebo or the total study population. The corresponding data is shown in Table 39.

TABLE 33
Percentage Change of IGA Scores From Baseline in The ITT Population and Normal
Eosinophil Population of Study Patients Receiving Oral DS107 and Placebo.

| | ITT Population | | | | | | | Normal Eosinophil Population | | | | |
|---|---|---|---|---|---|---|---|---|---|---|---|---|
| | | Baseline | Week 2 | Week 4 | Week 8 | Week 10 | | Baseline | Week 2 | Week 4 | Week 8 | Week 10 |
| Oral | Mean | 0 | −11.7 | −25.7 | −38.4 | −37.5 | Mean | 0 | −15.1 | −28.8 | −42.6 | −46.2 |
| DS107 | St DEV | 0 | 20.4 | 22.2 | 35.9 | 35.4 | | 0 | 23.7 | 24.6 | 37.7 | 37.5 |
| | SEM | 0 | 3.1 | 3.6 | 6.1 | 6.1 | | 0 | 4.6 | 4.8 | 7.4 | 7.4 |
| | n | 51 | 44 | 38 | 35 | 34 | | 26 | 26 | 26 | 26 | 26 |
| Placebo | Mean | 0 | −5.1 | −13.7 | −27.1 | −31.2 | Mean | 0 | −6.4 | −12.1 | −23.3 | −26.9 |
| | St DEV | 0 | 12.0 | 18.6 | 26.7 | 27.9 | | 0 | 13.1 | 18.0 | 28.6 | 30.3 |
| | SEM | 0 | 1.8 | 3.0 | 4.5 | 4.7 | | 0 | 2.2 | 3.0 | 4.8 | 5.1 |
| | n | 51 | 44 | 39 | 36 | 35 | | 35 | 35 | 35 | 35 | 35 |

TABLE 34
Percentage Change of EASI Scores From Baseline in The ITT Population and Normal
Eosinophil Population of Study Patients Receiving Oral DS107 and Placebo.

| | ITT Population | | | | | | | Normal Eosinophil Population | | | | |
|---|---|---|---|---|---|---|---|---|---|---|---|---|
| | | Baseline | Week 2 | Week 4 | Week 8 | Week 10 | | Baseline | Week 2 | Week 4 | Week 8 | Week 10 |
| Oral | Mean | 0 | −15.0 | −37.7 | −45.6 | −49.5 | Mean | 0.0 | −20.7 | −41.9 | −51.1 | −59.6 |
| DS107 | St DEV | 0 | 29.7 | 35.2 | 51.4 | 46.2 | | 0.0 | 30.3 | 36.4 | 42.1 | 38.1 |
| | SEM | 0 | 4.5 | 5.7 | 8.7 | 7.9 | | 0.0 | 5.9 | 7.1 | 8.3 | 7.5 |
| | n | 51 | 44 | 38 | 35 | 34 | | 26 | 26 | 26 | 26 | 26 |
| Placebo | Mean | 0 | −5.7 | −23.0 | −44.9 | −48.5 | Mean | 0.0 | −7.2 | −13.8 | −30.2 | −33.4 |
| | St DEV | 0 | 24.4 | 42.1 | 46.7 | 41.7 | | 0.0 | 23.3 | 52.3 | 64.9 | 55.4 |
| | SEM | 0 | 3.7 | 6.7 | 7.8 | 7.0 | | 0.0 | 3.9 | 8.8 | 11.0 | 9.4 |
| | n | 51 | 44 | 39 | 36 | 35 | | 35 | 35 | 35 | 35 | 35 |

TABLE 35
Percentage Change of VAS Scores From Baseline in The ITT Population and Normal
Eosinophil Population of Study Patients Receiving Oral DS107 and Placebo.

| | ITT Population | | | | | | | Normal Eosinophil Population | | | | |
|---|---|---|---|---|---|---|---|---|---|---|---|---|
| | | Baseline | Week 2 | Week 4 | Week 8 | Week 10 | | Baseline | Week 2 | Week 4 | Week 8 | Week 10 |
| Oral | Mean | 0 | −20.9 | −39.5 | −41.0 | −44.2 | Mean | 0.0 | −29.8 | −50.9 | −52.7 | −47.7 |
| DS107 | St DEV | 0 | 36.8 | 39.9 | 41.6 | 40.6 | | 0.0 | 41.1 | 39.5 | 40.0 | 45.8 |
| | SEM | 0 | 5.6 | 6.6 | 7.1 | 7.1 | | 0.0 | 8.1 | 7.7 | 7.8 | 9.0 |
| | n | 51 | 43 | 37 | 34 | 33 | | 26 | 26 | 26 | 26 | 26 |
| Placebo | Mean | 0 | −9.3 | −10.0 | −25.8 | −33.8 | Mean | 0.0 | −14.6 | −11.0 | −26.8 | −31.5 |
| | St DEV | 0 | 33.2 | 47.3 | 59.0 | 44.8 | | 0.0 | 26.9 | 48.0 | 55.4 | 43.6 |
| | SEM | 0 | 5.0 | 7.6 | 9.8 | 7.6 | | 0.0 | 4.5 | 8.1 | 9.4 | 7.4 |
| | n | 51 | 44 | 39 | 36 | 35 | | 35 | 35 | 35 | 35 | 35 |

TABLE 36
Percentage Change of SCORAD Levels From Baseline in The ITT Population and Normal
Eosinophil Population of Study Patients Receiving Oral DS107 and Placebo.

| | ITT Population | | | | | | | Normal Eosinophil Population | | | | |
|---|---|---|---|---|---|---|---|---|---|---|---|---|
| | | Baseline | Week 2 | Week 4 | Week 8 | Week 10 | | Baseline | Week 2 | Week 4 | Week 8 | Week 10 |
| Oral | Mean | 0 | −12.0 | −28.8 | −36.1 | −40.5 | Mean | 0.0 | −16.5 | −34.8 | −44.3 | −47.1 |
| DS107 | St DEV | 0 | 21.2 | 28.1 | 40.7 | 36.9 | | 0.0 | 23.2 | 24.8 | 38.1 | 36.7 |
| | SEM | 0 | 3.2 | 4.6 | 7.0 | 6.4 | | 0.0 | 4.5 | 4.9 | 7.5 | 7.2 |
| | n | 51 | 43 | 37 | 34 | 33 | | 26 | 26 | 26 | 26 | 26 |
| Placebo | Mean | 0 | −9.6 | −17.8 | −33.2 | −35.2 | Mean | 0.0 | −11.6 | −13.7 | −29.1 | −30.9 |
| | St DEV | 0 | 18.2 | 28.4 | 34.9 | 31.9 | | 0.0 | 16.1 | 29.8 | 38.9 | 33.8 |
| | SEM | 0 | 2.7 | 4.5 | 5.8 | 5.4 | | 0.0 | 2.7 | 5.0 | 6.6 | 5.7 |
| | n | 51 | 44 | 39 | 36 | 35 | | 35 | 35 | 35 | 35 | 35 |

TABLE 37
Percentage Change of BSA Levels From Baseline in The ITT Population and Normal
Eosinophil Population of Study Patients Receiving Oral DS107 and Placebo.

| | ITT Population | | | | | | | Normal Eosinophil Population | | | | |
|---|---|---|---|---|---|---|---|---|---|---|---|---|
| | | Baseline | Week 2 | Week 4 | Week 8 | Week 10 | | Baseline | Week 2 | Week 4 | Week 8 | Week 10 |
| Oral | Mean | 0 | −6.3 | −21.9 | −38.2 | −37.7 | Mean | 0.0 | −10.8 | −21.2 | −41.0 | −49.7 |
| DS107 | St DEV | 0 | 23.1 | 30.0 | 49.2 | 50.8 | | 0.0 | 23.2 | 33.5 | 47.4 | 48.3 |
| | SEM | 0 | 3.5 | 4.9 | 8.3 | 8.7 | | 0.0 | 4.5 | 6.6 | 9.3 | 9.5 |
| | n | 51 | 44 | 38 | 35 | 34 | | 26 | 26 | 26 | 26 | 26 |
| Placebo | Mean | 0 | −2.4 | −12.2 | −33.9 | −39.0 | Mean | 0.0 | −4.7 | −7.4 | −21.8 | −26.6 |
| | St DEV | 0 | 22.4 | 33.5 | 47.2 | 35.6 | | 0.0 | 18.0 | 49.4 | 68.2 | 55.5 |
| | SEM | 0 | 3.4 | 5.4 | 7.9 | 6.0 | | 0.0 | 3.0 | 8.4 | 11.5 | 9.4 |
| | n | 51 | 44 | 39 | 36 | 35 | | 35 | 35 | 35 | 35 | 35 |

TABLE 38
Percentage Change of DLQI Scores From Baseline in The ITT Population and Normal
Eosinophil Population of Study Patients Receiving Oral DS107 and Placebo.

| | ITT Population | | | | | | | Normal Eosinophil Population | | | | |
|---|---|---|---|---|---|---|---|---|---|---|---|---|
| | | Baseline | Week 2 | Week 4 | Week 8 | Week 10 | | Baseline | Week 2 | Week 4 | Week 8 | Week 10 |
| Oral | Mean | 0 | −29.5 | −50.3 | −43.2 | −53.6 | Mean | 0.0 | −41.3 | −58.2 | −57.5 | −61.6 |
| DS107 | St DEV | 0 | 48.0 | 36.7 | 43.6 | 41.3 | | 0.0 | 47.1 | 38.7 | 40.2 | 42.0 |
| | SEM | 0 | 7.2 | 6.0 | 7.4 | 7.1 | | 0.0 | 9.2 | 7.6 | 7.9 | 8.2 |
| | n | 51 | 44 | 38 | 35 | 34 | | 26 | 26 | 26 | 26 | 26 |
| Placebo | Mean | 0 | −22.3 | −31.9 | −34.4 | −42.2 | Mean | 0.0 | −32.5 | −39.8 | −39.9 | −44.3 |
| | St DEV | 0 | 43.8 | 37.8 | 47.5 | 36.5 | | 0.0 | 36.5 | 34.3 | 49.3 | 34.6 |
| | SEM | 0 | 6.7 | 6.1 | 7.9 | 6.2 | | 0.0 | 6.2 | 5.8 | 8.3 | 5.9 |
| | n | 51 | 43 | 38 | 36 | 35 | | 35 | 35 | 35 | 35 | 35 |

TABLE 39
Percentage Change of POEM Scores From Baseline in The ITT Population and Normal
Eosinophil Population of Study Patients Receiving Oral DS107 and Placebo.

| | ITT Population | | | | | | | Normal Eosinophil Population | | | | |
|---|---|---|---|---|---|---|---|---|---|---|---|---|
| | | Baseline | Week 2 | Week 4 | Week 8 | Week 10 | | Baseline | Week 2 | Week 4 | Week 8 | Week 10 |
| Oral | Mean | 0 | −30.2 | −41.8 | −37.2 | −43.4 | Mean | 0.0 | −41.8 | −55.3 | −53.1 | −50.1 |
| DS107 | St DEV | 0 | 33.3 | 35.6 | 40.7 | 38.2 | | 0.0 | 34.3 | 33.0 | 35.9 | 38.7 |
| | SEM | 0 | 5.0 | 5.8 | 6.9 | 6.6 | | 0.0 | 6.7 | 6.5 | 7.0 | 7.6 |
| | n | 51 | 44 | 38 | 35 | 34 | | 26 | 26 | 26 | 26 | 26 |
| Placebo | Mean | 0 | −15.3 | −17.1 | −22.0 | −28.3 | Mean | 0.0 | −22.5 | −19.7 | −29.1 | −34.4 |
| | St DEV | 0 | 39.9 | 43.8 | 56.2 | 55.1 | | 0.0 | 29.2 | 48.7 | 49.1 | 38.1 |
| | SEM | 0 | 6.0 | 7.0 | 9.4 | 9.3 | | 0.0 | 4.9 | 8.2 | 8.3 | 6.4 |
| | n | 51 | 44 | 39 | 36 | 35 | | 35 | 35 | 35 | 35 | 35 |

As mentioned in Example 2, a surprising discovery was observed when the data was further interrogated. Here, another surprising discovery was observed when the data was stratified in a post hoc analysis. In particular, it was found that subjects having normal eosinophil counts of less than about 0.3×10⁹/L exhibited a higher response rate to DS107 treatment.

Results of the analyses are shown in FIGS. 36-43B and Tables 30-39 as discussed above. The data shows an increase in responder rates as measured by IGA, pruritus (VAS), EASI, SCORAD, BSA, DLQI and POEM scores in subjects having normal eosinophil counts compared to the total study population. In particular, 42% of DS107-treated patients having normal eosinophil levels were classified as responders using IGA scoring, versus only 22% of the same population receiving placebo. FIGS. 36-37B and Tables 30, 31, 32 and 33. As discussed in detail above, a subject is classified as a Responder if he/she achieves an IGA score of 0 (clear) or 1 (almost clear) at Week 8, considering a 2-point decrease from baseline. FIG. 38A-38B depicts similar results using EASI scoring where 51% of the DS107-treated patients having normal eosinophil levels were classified as responders compared to only 30% of the same population receiving placebo. Tables 30, 31, 32 and 34. Pruritus scores (as measured by VAS) showed a similar pattern, with 53% of DS107-treated patients having normal eosinophil levels classified as responders compared to 27% of the same population receiving placebo. FIGS. 39A-39B and Tables 30, 31, 32 and 35. Similar results are reflected for SCORAD levels, BSA scores, DLQI scores, and POEM scores. FIGS. 40A-43B and Tables 30, 31, 32 and 35-39.

Together these data suggest that patients pre-determined to have normal eosinophil levels represent likely candidates for treatment with DGLA, in particular an oral DGLA such as DS107. Contrary to current practices, which report more efficacious pharmaceutical-based therapies in patients having high eosinophil levels (e.g., greater than about 0.3×10⁹/L), the findings described herein are useful in predicting efficacy of a DGLA therapy for patients.

1. A method of treating one or more conditions in a subject having a normal eosinophil count, the method comprising administering pharmaceutical composition comprising DGLA or a derivative thereof to the subject.

2. The method of claim 1, wherein the subject is an adult subject.

3. The method of claim 1, wherein the subject is a pediatric subject.

4. The method of claim 1, wherein the eosinophil count is determined based on a comparison to a laboratory reference level.

5. The use of claim 1, wherein the normal eosinophil count is less than about 0.2×10⁹/L, less than about 0.25×10⁹/L, less than about 0.3×10⁹/L, less than about 0.4×10⁹/L, less than about 0.5×10⁹/L, less than 0.6×10⁹/L, less than 0.7×10⁹/L or less than about 0.8×10⁹/L.

6. The method of claim 1, wherein the one or more conditions are atopic conditions.

7. The method of claim 1, wherein the one or more conditions include chronic obstructive pulmonary disease (COPD), vasculitides, asthma, chronic rhinosinusitis, or a combination thereof.

8. (canceled)

9. The method of claim 7, wherein vasculitides includes large vessel vasculitis (LVV), medium vessel vasculitis (MVV), small vessel vasculitis (SVV), variable vessel vasculitis (VVV), single-organ vasculitis (SOV) and vasculitis associated with systemic disease.

10. (canceled)

11. The method of claim 7, wherein the chronic rhinosinusitis includes chronic rhinosinusitis with nasal polyps (CRSwNP).

12. The method of claim 11, wherein nasal polyps (CRSwNP) include a non-eosinophilic subtype.

13. The method of claim 6, wherein the atopic conditions include atopic dermatitis.

14. The method of claim 13, wherein the DGLA or derivative thereof reduces at least one of an investigator global assessment (IGA) level, eczema area and severity index (EAST), a percentage of area of an anatomical site affected by atopic dermatitis, a scoring atopic dermatitis (SCORAD), a body surface area affected by atopic dermatitis, a dermatology life quality Index (DLQI), a patient-oriented eczema measure (POEM), or visual analog scale (VAS) pruritus score.

15. The method of claim 14, wherein the EASI score decreases by about 30%, about 40%, about 50%, about 60%, about 70% or more than about 70% compared to baseline, wherein the VAS score decreases b about 30%, about 40%, about 50%, about 60% or more than about 60% compared to baseline, wherein the SCORAD score decreases by about 30%, about 40%, about 50%, about 60%, about 70% or more than about 70% compared to baseline, wherein the IGA score decreases by about 30%, about 40%, about 50%, about 60% or more than about 60% compared to baseline, wherein the DLQI score decreases by about 40%, about 50%, about 60%, about 70%, or more than about 70% compared to baseline, and/or wherein the POEM score decreases by about 40%, about 50%, about 60%, about 70%, or more than about 70% compared to baseline.

16.-18. (canceled)

19. The method of claim 14, wherein the body surface area affected by atopic dermatitis is assigned a body surface area (BSA) score.

20. The method of claim 19, wherein the BSA score decreases by about 30%, about 40%, about 50%, about 60% or more than about 60% compared to baseline.

21.-22. (canceled)

23. The method of claim 14, wherein a pruritus level and/or a pruritus delta are calculated from the VAS.

24. The use method of claim 23, wherein pruritus level is reduced by about 30%, about 40%, about 50%, about 60%, about 70%, about 80%, about 90% or more than about 90% compared to baseline.

25. The method of claim 23, wherein pruritus delta is about 30%, about 40%, about 50%, about 60%, about 70%, or more than about 70%.

26. The method of claim 6, wherein atopic dermatitis includes atopic dermatitis having any EASI, VAS, SCORAD, IGA, BSA, DLQI, and/or POEM score.

27. The method of claim 1, wherein the one or more conditions include chronic obstructive pulmonary disease (COPD), asthma, atopic dermatitis, chronic rhinosinusitis, or a combination thereof.

28. The method of claim 1, wherein the subject is a member of a group having eosinophil levels that are greater than normal eosinophil levels, less than normal eosinophil levels, or a combination thereof.

29. The method of claim 1 wherein the DGLA or a derivative thereof is in a liquid or semi-liquid format.

30. The method of claim 1 wherein the DGLA or a derivative thereof is in a capsule.

31. The method of claim 13 wherein the DGLA or a derivative thereof is in a liquid or semi-liquid format.

32. The method of claim 13 wherein the DGLA or a derivative thereof is in a capsule.
